(19) United States
(12) Patent Application Publication
(10) Pub. No.: US 2022/0064672 A1
(43) Pub. Date: Mar. 3, 2022

(54) ENGINEERED ONCOLYTIC VIRUSES EXPRESSING PD-L1 INHIBITORS AND USES THEREOF

(71) Applicant: University of Southern California, Los Angeles, CA (US)

(72) Inventors: Siyi CHEN, Los Angeles, CA (US); Xue F. HUANG, Los Angeles, CA (US); Guan WANG, Los Angeles, CA (US)

(73) Assignee: University of Southern California, Los Angeles, CA (US)

(21) Appl. No.: 17/458,909

(22) Filed: Aug. 27, 2021

Related U.S. Application Data
(60) Provisional application No. 63/071,159, filed on Aug. 27, 2020.

(51) Int. Cl. C12N 15/86; C12N 7/00; C07K 14/705; C07K 14/535; A61P 35/00; A61K 9/00; A61K 45/06; A61K 38/17; A61K 38/19; C12N 5/0783

(52) U.S. Cl. CPC C12N 15/86; C12N 2710/24132; C07K 14/70532; C07K 14/70521; C07K 14/535; A61P 35/00; A61K 9/0019; A61K 45/06; A61K 38/1774; A61K 38/193; C12N 5/0636; C12N 2810/859; C12N 2710/24143; C12N 2710/24171; C12N 2710/24121; C07K 2319/30; C12N 7/00

(57) ABSTRACT

Oncolytic viruses offer an in situ vaccination approach to activate tumor-specific T cell responses. However, the upregulation of PD-L1 expression on tumor cells and immune cells leads to tumor resistance to oncolytic immunotherapy. Herein, we generate an engineered oncolytic virus that coexpresses a PD-L1 inhibitor and GM-CSF. This oncolytic virus is capable of secreting the PD-L1 inhibitor that systemically binds and inhibits PD-L1 on tumor cells and immune cells. The intratumoral injection with the oncolytic virus overcomes PD-L1-mediated immunosuppression during both the priming and effector phases, provokes systemic T cell responses against dominant and subdominant neoantigen epitopes derived from mutations, and leads to an effective rejection of both virus-injected and distant tumors. This engineered oncolytic virus allows for activation of tumor neoantigen-specific T cell responses, providing a potent, individual tumor-specific oncolytic immunotherapy for cancer patients, especially those resistant to PD-1/PD-L1 blockade therapy.

### CROSS-REFERENCE TO RELATED APPLICATIONS

This application includes a claim of priority under 35 U.S.C. § 119(e) to U.S. provisional patent application No. 63/071,159, filed Aug. 27, 2020, the entirety of which is hereby incorporated by reference.

### REFERENCE TO SEQUENCE LISTING

The Sequence Listing submitted Aug. 27, 2021 as a text file named “SequenceListing-065715-000116US00_ST25” created on Aug. 26, 2021 and having a size of 21,588 bytes, is hereby incorporated by reference.

### FIELD OF INVENTION

This invention relates to anti-tumor therapies, and specifically those involving oncolytic viruses.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Cancer is a genetic disease, with the growth of tumor cells initiated by mutations that activate oncogenic drivers and disable tumor suppressors. Recent studies have demonstrated that tumor neoantigens can be derived de novo from the expression of genetic mutations and presented in major histocompatibility complexes (MHC) on tumor cells, and endogenous T cell responses against neoantigens can be naturally activated in cancer patients. However, only a small number of nonsynonymous mutations expressed in tumors can be adequately presented as neoantigens for which the T cell response can be mounted.

T cells against mutant neoantigens that are individually tumor specific play an important role in driving antitumor immunity. Each tumor harbors a unique repertoire of mutated neoantigenic peptides that are immunogenic and can potentially induce tumor-specific immune responses. T cells can be activated against shared, nonmutated tumor-associated self-antigens. NK cells and NKT cells also have antitumor activities. Thus far, the majority of cancer patients still fail to spontaneously activate neoantigen-specific T cells and are resistant to immune checkpoint blockade therapy, likely due to the poor presentation of tumor neoantigens and the immunosuppressive tumor microenvironment.

Compounding this problem of inefficient neoantigen presentation is the immunosuppressive tumor microenvironment that inhibits antitumor T cell responses by immune checkpoint molecules, such as programmed cell death protein 1 (PD-1) and programmed death-ligand 1 (PD-L1). Immune checkpoint blockade effectively augments endogenous T cell responses against tumor neoantigens and led to the enduring responses in patients with advanced malignancies, including complete responses in various types of cancer, such as melanoma, metastatic lung, kidney, and bladder carcinoma. Responses to PD-1 inhibition are highly correlated with the presence of CD8+ T cells at the invasive margin and within the tumor lesions, which define the so-called inflamed “hot” tumors. However, the majority of cancer patients are resistant to PD-1/PD-L1 blockade. One reason for treatment failures is attributed to the so-called “cold” tumors, which might have low mutational burden and neoantigen load, poor MHC presentation, and poor capacity to attract T cell infiltration. Increasing the response rates to PD-1 blockade therapy remains an important challenge, given that the majority of tumors fail to spontaneously provoke T cell responses against tumor mutant neoantigens and are resistant to PD-1 blockade. Recently, intensive efforts have been devoted to activating neoantigen-specific T cell responses. Neoantigen-specific T cells can be activated by comprehensive sequencing and the identification of individual mutations, the computational prediction of neoantigen epitopes, and vaccination with neoantigen epitopes for each patient.

It is an objective to provide compositions or therapies including oncolytic viruses that can mitigate or circumvent tumor resistance to oncolytic immunotherapy.

It is another objective to provide methods of immunotherapy treatment to patients and/or methods of preparation of oncolytic viruses to afford immunotherapies that combats tumor resistance.

### SUMMARY OF THE INVENTION

A recombinant oncolytic virus expressing an inhibitor of programmed death-ligand 1 (PD-L1) or an inhibitor of programmed cell death protein 1 (PD-1) and granulocyte-macrophage colony-stimulating factor (GM-CSF) by introduction of genes is provided, wherein the recombinant oncolytic virus is introduced with a polynucleotide encoding the inhibitor of PD-L1 or the inhibitor of PD-1 and the GM-CSF, or wherein the recombinant oncolytic virus is introduced with a set of polynucleotides including a first polynucleotide encoding the inhibitor of PD-L1 or the inhibitor of PD-1 and a second polynucleotide encoding the GM-CSF.

In some implementations, the recombinant oncolytic virus is a vaccinia virus, a herpes simplex virus, a reovirus, a vesicular stomatitis virus, a poliovirus, a senecavirus, or a Semliki Forest virus.

In some implementations, the recombinant oncolytic virus is a vaccinia virus, wherein the vaccinia virus has one or both thymidine kinase and vaccinia growth factor viral gene deleted from its backbone or inactivated.

In various implementations, the one or more polynucleotides encode, in expressible form, the polypeptide inhibitor of PD-L1 and the GM-CSF, wherein the polypeptide inhibitor of PD-L1 is a fusion protein comprising an extracellular domain of programmed cell death protein 1 (PD-1) and a crystallizable fragment of immunoglobulin class G (IgG-Fc). In some aspects, the one or more polynucleotides encode a polypeptide inhibitor of human PD-L1 and human GM-CSF. In some other aspects, the one or more polynucleotides encode a polypeptide inhibitor of mouse PD-L1 and mouse GM-CSF. For example, the one or more polynucleotides include a first nucleic acid sequence of SEQ ID NO:51, which encodes a polypeptide inhibitor having an amino acid sequence of SEQ ID NO:52, which is an inhibitor of human PD-L1, and a second nucleic acid sequence of SEQ ID NO:53, which encodes the human GM-CSF having an amino acid sequence of SEQ ID NO:54. In another example, the polynucleotide encoding for an inhibitor of mouse PD-L1 has a nucleic acid sequence of SEQ ID NO:1, which encodes a fusion protein of SEQ ID NO:2 comprising at least a portion of mouse PD-1 and IgG-Fc.

In various implementations, the recombinant oncolytic virus induces apoptosis of a tumor cell resistant to GM-CSF, resistant to an oncolytic virus without a nucleic acid sequence encoding any of the polypeptide inhibitor of PD-L1, the polypeptide inhibitor of PD-1, and the GM-CSF, or resistant to both the GM-CSF and the oncolytic virus with the nucleic acid sequence.

A system, comprising the recombinant oncolytic virus and a mammalian cell, is also provided, wherein upon infecting the mammalian cell with the recombinant oncolytic virus, the mammalian cell secretes the inhibitor encoded by the one or more polynucleotides introduced into the recombinant oncolytic virus.

Sera isolated from a mammal infected with the recombinant oncolytic virus are also provided, wherein the recombinant oncolytic comprises one or more nucleic acid sequences encoding a polypeptide inhibitor of PD-L1 or a polypeptide inhibitor of PD-1 and granulocyte-macrophage colony-stimulating factor (GM-CSF), wherein the serum contains the polypeptide inhibitor of PD-L1 or the polypeptide inhibitor of PD-1.

Methods of treating a subject suffering from cancer are also provided, including administering to the subject an effective amount of the recombinant oncolytic virus to induce infiltration of one or more T cells into the cancer. In various aspects, the methods are effective for inhibiting the growth or reducing the size of the tumor. In further aspects, the methods are also effective for inhibiting the growth or reducing the size of a distant tumor in the subject; or reducing tumor metastases.

In some instances, the cancer comprises adenoma, melanoma, neoplasm of mammary, pancreatic cancer, glioblastoma, colorectal cancer, or a combination thereof. In some aspects, the subject in the methods has an existing tumor or the subject has a reoccurring tumor. In further aspects, the subject's response to a therapy of an inhibitor of PD-1, an inhibitor of PD-L1, or both is ineffective. In another aspect, the subject's response to a therapy consisting of an inhibitor of PD-1, an inhibitor of PD-L1, an inhibitor of PD-1 and an inhibitor of PD-L1, or an inhibitor of PD-1 and/or an inhibitor of PD-L1 and a pharmaceutically acceptable excipient or carrier is ineffective. In some aspects, the subject's splenic T cells are responsive to tumor neoantigens for 10 days, 20 days, 30 days, 40 days, or longer, after the administration.

In some implementations, the composition is administered intratumorally or the composition is delivered into the tumor. In some other implementations, the composition is administered via a parenteral route.

In some implementations, the methods further include administering to the subject an additional therapeutic agent, e.g., an inhibitor of PD-1, an inhibitor of PD-L1, a chemotherapeutic agent, or a combination thereof.

Additional methods are provided for treating a subject suffering from cancer, which includes administering to the subject an effective amount of the recombinant oncolytic virus to induce infiltration of one or more T cells into the cancer, isolating the tumor-infiltrated T cells from the cancer of the subject, expanding the tumor-infiltrated T cells ex vivo, and transferring the expanded tumor-infiltrated T cells to the same subject suffering from the cancer or to another subject in need thereof.

Methods for generating tumor-infiltrating, oncolytic virus-induced T cells, are also provided, which include administering, to a subject having a cancer, an effective amount of the composition comprising the recombinant oncolytic virus of claim 1, to induce infiltration of one or more T cells into the cancer, resulting in tumor-infiltrated T cells; and isolating the tumor-infiltrated T cells from the cancer of the subject. In further implementations, the methods for generating tumor-infiltrating, oncolytic virus-induced T cells further include expanding the tumor-infiltrated T cells ex vivo.

In further implementations, a method of treating or reducing severity of cancer in a subject includes administering to the subject an effective amount of a composition comprising the serum obtained from a mammal infected with the recombinant oncolytic virus so as to induce infiltration of one or more T cells in the cancer, thereby treating or reducing severity of the cancer in the subject.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-1K depict generation and characterization of an oncolytic vaccinia virus coexpressing a mouse PD-L1 inhibitor and GM-CSF. (FIG. 1A) A schematic diagram of recombinant vaccinia virus (VV) shuttle vectors that express GM-CSF or/and iPDL1 (soluble PD-1-Fc). vTK, VV thymidine kinase gene; R and L, right and left flank sequences; RFP, red fluorescent protein. (FIG. 1B) Expression and secretion of iPDL1 from infected MC38 tumor cells (a line derived from C57BL6 murine colon adenocarcinoma cells) infected with the indicated VVs. Anti-IgG Fc (Licor 926-32210; upper) or anti-PD-1 (Biolegend 114101; lower) was used for western blot with reducing or non-reducing loading buffer. The experiment was repeated twice. (FIG. 1C) Serum iPDL1 and GM-CSF levels in different VV-treated MC38-bearing mice at 2 days post-virus injection. (FIG. 1D) Kinetics of iPDL1 levels in injected tumors or sera of the VV-iPDL1/GM-treated mice. n=3 independent samples. Data are presented as the means±SD. The experiment was repeated twice. (FIGS. 1E, 1F) Purified iPDL1 binds to PD-L1+ tumor cell. FIG. 1E: flow cytometric analysis of PD-L1 expression on shPD-L1/MC38 tumor cells that were transduced with PD-L1-shRNA and wild-type MC38 cells. FIG. 1F: shPD-L1/MC38 cells and wild-type MC38 cells were incubated with 50 μg/mL of purified iPDL1, an irrelevant MAGE3-IgG Fc fusion protein, or IgG control, followed by staining with an anti-IgG Fc for flow cytometry. (FIG. 1G) Inhibition of PD-1/PD-L1 binding by purified iPDL1 protein using ELISA. An anti-PD-L1 antibody was used as a positive control; n=3 independent samples. (FIG. 1H) iPDL1-mediated ADCC. ADCC Reporter Bioassays were performed in triplicate wells, and the concentrations of iPDL1 protein and control IgG Fc used for this assay are indicated; n=3 independent samples. Data presented as the means±SD. The experiment was repeated twice. Significant differences are indicated as ***P<0.001, or ****P<0.0001 using two-tailed student's t-test. (FIG. 1I) CD11c+ DC frequency in monocyte cultures in the presence of culture media of MC38 cells infected with VV-RFP, VV-GM, VV-iPDL1/GM, or GM-CSF as a positive control, and IL-4. (FIG. 1J) Viral replication in vitro; n=3 independent samples. (FIG. 1K) Replication and biodistribution of VV after intratumor injections. Data presented as the means±SD. The experiment was repeated twice.

FIG. 1L depicts efficient secretion of GM-CSF and PD-L1 inhibitors (iPDL1) from VV-iPDL1/GM-infected tumor cells. B16-F10, Py230, or MC38 tumor cells were infected with VV-RFP or VV-iPDL1/GM at an MOI=2. 24, 48, or 72 h after VV infection, supernatants were collected and clarified. iPDL1 (PD1-Fc) was measured using mouse PD1 DuoSet ELISA kit (R&D, DY1021) and GM-CSF measured using mouse GM-CSF ELISA kit (Biolegend, Cat #432204). n=3 independent samples. Data presented as the means±SD. The experiment was repeated twice.

FIG. 1M depicts oncolytic activities of the recombinant virus VV-iPDL1/GM. On the left, oncolytic activity of VVs against different tumor cells. Mouse tumor cells (B16-F10, Py230, MC38) were infected with the indicated VVs at an MOI of 1 or 5 for 24, 48, 72, and 96 h. MTT assay were performed to determine viability of infected tumor cells. The percentages of viable cells are presented at different time points. n=3 independent samples. Data are presented as the means±SD. Experiments were repeated twice. On the right, VV efficiently infected MC38 tumor cells, but not EL-4 (lymphoma-derived) tumor cells. Tumor cells seeded in 24-well plate were infected with the indicated MOI of VV-iPDL1/GM. 48 h later, the cells were harvested and analyzed by flow cytometry. RFP positive cells represent infected cells. Experiments were repeated three times.

FIG. 2A-2G depict PD-L1 inhibitors secreted from VV-iPDL1/GM-infected cells bind to PD-L1 on tumor cells and immune cells. (FIG. 2A) MC38 tumor cells were infected with VV-RFP, VV-iPDL1/GM at an MOI=0.5, or PBS for 24 h. The percentage of IgG Fc+ population representing iPDL1 (soluble PD-1-IgG Fc)-bound VV-infected (RFP+) or uninfected (RFP−) PD-L1-expressing tumor cells was measured by flow cytometry. (FIG. 2B) MC38 tumor cells that were stimulated with IFN-γ (20 ng/mL) for 48 h were infected with the indicated VVs. PD-L1 expression of infected (RFP+) or uninfected (RFP−) cells was determined by flow cytometry. (FIG. 2C-2G) MC38 cells were subcutaneously inoculated into the left (1×10⁶) and right (5×10⁵) flanks of C57BL/6 mice. When left flank tumor sizes reached ˜100 mm³ (counted as day 0), the tumors of the left flank were intratumorally injected with 50 μL of PBS, VV-RFP, VV-GM, or VV-iPDL1/GM (5×10⁷ pfu per tumor), or 200 μg of anti-PD-L1 antibody (clone 10F.9G2) intravenously on days 0 and 3. Two days post-second VV treatment, VV-treated (primary tumor) and untreated, distant tumors were collected, weighed and digested with collagenase type I and DNase. Tumor cell suspensions were blocked with anti-CD16/32 antibody and then stained with antibodies against CD45, CD3, CD8, CD4, CD11c, CD11b, Gr-1, FoxP3, PD-L1, and IgG Fc to assess PD-L1 expression or IgG Fc+ frequency on infected (RFP+) or uninfected (RFP−) tumor cells from the treated primary tumors (FIG. 2C) or untreated distant tumors (CD45− cells) (FIG. 2D), and PD-L1 expression on infiltrating immune cells from treated (FIG. 2E) or untreated distant tumors (FIG. 2F), IgG Fc+ frequency on infiltrating immune cells (FIG. 2G). Infiltrating immune cells include cytotoxic T cells (CD45+CD3+CD8+), DCs (CD45+CD11c+), myeloid-derived suppressor cells (MDSCs) (CD45+CD11c⁻CD11b+Gr−1+), and Treg (CD45+CD3+CD4+FoxP3+); n=5 mice. Significant differences are indicated as **P<0.01, ***P<0.001, or ****P<0.0001 determined by two-tailed Student's t-test.

FIG. 2H depicts representative tumor growth rates prior to virus treatment. C57BL/6 mice were implanted with 5 ×10⁵ B 16-F 10 (melanoma), Py230 (Malignant neoplasms of mammary), or MC38 (colon adenocarcinoma) cells subcutaneously. Tumor volume was monitored by caliper measurement on indicated days. n=5 mice. Data presented as the means±SD.

FIG. 2I depicts both CD45 positive and negative cells in tumor microenvironment were infected with intratumorally injected viruses. Groups of C57BL/6 mice were subcutaneously inoculated with MC38 cells (1×10⁶). When tumor sizes reached ˜100 mm³ (counted as day 0), the tumors were intratumorally injected with 50 μL of PBS, VV-RFP, VV-GM or VV-iPDL1/GM (5×10⁷ pfu per tumor) on days 0 and 3. Two days post-2nd viral injection, VV-treated tumors were collected, weighed and digested with collagenase type I and DNase. A fraction of the tumor cell suspensions were blocked with anti-CD16/32 antibody and then stained with antibodies against CD45 to assess infected cells (RFP+) frequency of CD45 positive and negative cells.

FIG. 2J depicts tumor purity was confirmed by flow cytometry after kit isolation. Using the same treatment schedule as in FIG. 2I, tumor cells were isolated from the other fraction of the tumor cell suspensions using a tumor cell isolation kit (Miltenyl Biotec, Cat #130-110-187). The isolated tumor cells and tumor cell suspensions before isolation were stained with lineage markers anti-CD45, CD31, and Ter119 antibodies.

FIG. 2K depicts tumor cells were infected by injected viruses. Using the same treatment schedule as in FIG. 2J, VV-infected cells (RFP+) frequencies of isolated tumor cells were analyzed by flow cytometry.

FIG. 2L depicts infected tumor cells were able to secrete iPDL1. Using the same treatment schedule as in FIG. 2J, isolated tumor cells were further cultured in vitro for 48 h. Supernatants of the culture media and tumor cell lysates were analyzed by Western Blot using an anti-IgG Fc (Licor 926-32210) with reducing or non-reducing loading buffer. The experiment was repeated twice.

FIG. 2M depicts secreted iPDL1 from infected isolated tumor cells binds to immune cells. Mature bone marrow-derived dendritic cells (BMDC) were pre-treated with anti-CD16/32 antibodies to block FcRs binding on ice for 30 min. Cells were then incubated with the supernatants of the tumor cell culture media in FIG. 2L, followed by stained anti-IgG-Fc or isotype IgG control. Mature BM-DC were also separately stained with anti-PD-L1.

FIG. 3A-3D depict enhanced antitumor activities against primary tumors. (FIG. 3A-3B) C57BL/6 mice were subcutaneously inoculated with 5×10⁵ luciferase-expressing B16-F10 (B16-F10-Luc) cells. When tumor sizes reached ˜100 mm³ (counted as day 0), the mice were intratumorally injected with 50 μL of VV-RFP, VV-GM, or VV-iPDL1/GM (5×10⁷ pfu per tumor) or PBS at days 0, 3, and 7. Bioluminescence monitoring as shown in FIG. 3A and caliper measurement as shown in FIG. 3B of B16-F10-Luc cells were performed on the indicated days. Data are presented as the means±SD (n=5 mice). Significant differences are indicated as *P<0.05 determined by two-tailed Student's t-test. (FIGS. 3C, 3D) Py230 (FIG. 3C) or MC38 (FIG. 3D) tumor volume was monitored by caliper measurement using the same treatment schedule as in FIG. 3A-3B. Data are presented as the means±SD (n=5 mice).

FIG. 4A-4K depict enhanced antitumor activities against untreated, distant tumors. (FIG. 4A-4C) Inhibition of rechallenged tumor growth. B16-F10 melanoma cells were implanted intradermally to the left flank of C57B/6 mice. When tumor sizes reached ˜100 mm³ (counted as day 0), the mice were intratumorally injected with the indicated VVs on days 0, 3, and 7. Treated mice were s.c. rechallenged with B16-F10-Luc cells 30 days after the last VV injection (counted as day 0 for rechallenge data). Bioluminescence monitoring as quantified in FIG. 4A and caliper measurement of B16-F10-Luc cells as shown in FIG. 4B were performed. Data are presented as means±SD (n=5 mice). (FIG. 4C) Survival curve of B16-F10 rechallenged mice. (FIG. 4D-4G) The volumes of rechallenged Py230 (FIG. 4D) or MC38 (FIG. 4F) tumors were monitored using a similar treatment schedule as in FIG. 4A (days 10, 15, 20, and 25), except that 5×10⁵ of Py230 or MC38 tumor cells were rechallenged. Data are presented as means±SD (n=5 mice). *P<0.05, ***P<0.001 determined by two-way ANOVA. Survival curve of Py230 (FIG. 4E) and MC38 (FIG. 4G) rechallenge mice. *P<0.05, *** P<0.001 by two-tailed Log rank test. (FIG. 4H) CD8 T cell depletion. Surviving mice treated with VV-iPDL1/GM for the original left flank tumor implantation were rechallenged with 5×10⁵ MC38 cells at right side without or with weekly i.v. injections of anti-CD8 antibody for two times. Data are presented as means±SD (n=5 mice). ****P<0.01 by two-tailed repeated-measures two-way ANOVA. (FIG. 4I-4K) Inhibition of untreated, established tumor growth. B16-F10 melanoma cells were implanted to the left and right flanks of C57B/6 mice. The mice were intratumorally injected to the left flank tumors with indicated VVs without or with i.v. injections of anti-PD-L1 antibody on days 0, 3, and 7. (FIG. 4I) Individual curves are depicted for each tumor. Numbers indicate complete tumor regression out of total tumors in each group. (FIG. 4J) Distribution of tumor volumes determined on day 30 after virus injection; n=10 mice. Bars represent mean values±SD. *P<0.05 by two-tailed Mann-Whitney U test. (FIG. 4K) Cumulative survival curves. Data are from two independent experiments. *P<0.05; **P<0.01; NS, not significant by two-tailed Log rank test.

FIG. 4L depicts comparison of antitumor activities of VV-iPDL1 with the coadministrations of VV-GM and IgG Fc. B16-F10 melanoma cells were implanted to the left and right flanks of C57B/6 mice (5×10⁵ to the left flank and 1×10⁵ to the right flank). When the volume of left flank tumors reached ˜100 mm³ (counted as day 0), the mice were intratumorally injected with 50 μL of IgG-Fc (100 μg/tumor, Thermo, 31205) only, VV-GM (5 ×10⁷ pfu/tumor), VV-GM (5×10⁷ pfu/tumor)+IgG-Fc (100 μg/tumor) (premixed and injected together), VViPDL1/GM (5×10⁷ pfu/tumor), or PBS three times on days 0, 3, and 7. The left (primary tumor location, directly injected with treatment) and right (distant tumor location, not injected) flank tumor sizes were measured every 3-5 days. n=5 mice. *P<0.05 by repeated measures 2-way ANOVA. The experiment was repeated once.

FIG. 4M depicts distribution of tumor volumes on day 10 after virus injection. Similar with B16-F10 tumor establishment in FIG. 3A, the mice were intratumorally injected with 50 μL of PBS, VV-GM, VV-iPDL1/GM (5×10⁷ pfu per tumor) and 200 μg/mouse anti-PDL1 antibody intravenously each time on day 0, 3, and 7. n=10 mice. Bars represent mean values±SD. *P<0.05 by two-tailed Mann-Whitney U test. The experiment was repeated once.

FIG. 5A-5C depict enhanced tumor infiltration and activation of immune cells. A similar treatment schedule as in FIG. 2C was used, except that 5 days after the second VV injection, VV-treated MC38 tumors were harvested, weighed, and digested for preparation of single-cell suspensions followed by antibody staining against CD45, CD8, CD4, CD11c, CD11b, Gr-1, and FoxP3. (FIG. 5A-5B) Absolute numbers of infiltrating CD45+ immune cells, DCs, MDSCs, CD4+ T cells, CD8+ T cells, and Tregs and CD8+ T cell/Treg ratio values in treated tumors (FIG. 5A) and distant, untreated tumors (FIG. 5B). n=5 mice. Data presented as the means±SD. *P<0.05, **P<0.01 by two-tailed Student's t-test. (FIG. 5C) Quantitative presentation of expression of IFN-γ, TNF-α, and CD 107a of tumor-infiltrating CD8+ T cells in response to restimulation with MC38 tumor lysate-pulsed DCs in the presence of Golgi-plug for 8 h were measured by intracellular staining. n=5 mice. Data presented as the means±SD. *P<0.05 by two-tailed Student's t-test.

FIG. 5D depicts PD-1 expression of CD8+ T cell in virus-treated tumors. A similar treatment schedule as in FIG. 5A was used, VV-treated MC38 tumors were harvested, weighed and digested for preparation of single cell suspensions followed by antibody staining against CD45, CD3, CD8, or PD-1.

FIG. 6A-6F depict enhanced T cell responses against dominant and subdominant tumor neoantigen epitopes. (FIG. 6A) Enhanced T cell responses against a pool of neoantigen peptides. MC38 tumor-bearing mice were intratumorally injected with various VVs at days 0, 3, and 7. One group of C57BL/6 mice were i.v. injected with 200 μg of anti-PD-L1 antibody. Ten days later, splenocytes were cultured in the presence of a mixture of 11 neoepitope peptides (10 μg/mL/each). After 80 h of incubation, supernatants were collected for IFN-γ ELISA (right). [3H] thymidine incorporation was measured (left). The graph shows the results from three mice of each group. Data presented as the means±SD. *P<0.05 by two-tailed Student's t-test. (FIG. 6B) Enhanced T cell responses against individual neoantigens. The splenocytes from VV-treated mice were cocultured with each of the 11 neoepitope peptides (100 μg/ml) as above described above. [3H] thymidine incorporation (left) and ELISA IFN-γ concentrations (right) are shown; n=3 mice. One bar or one dot represents one mouse. Data presented as the means±SD. *P<0.05 by two-tailed Student's t-test. (FIG. 6C) Enhanced T cell responses against the neoantigenic peptide 11. The splenocytes isolated from VV-treated mice were cocultured with various concentrations of the neoepitope peptide 11 as above described above. [3H] thymidine incorporation was used to analyze T cell proliferation; n=3 mice. Data presented as the means±SD. *P<0.05, **P<0.01, ***P<0.001 by two-tailed Student's t-test. (FIG. 6D, FIG. 6E) Enhanced tumor infiltration of neopeptide 4-specific T cells. Tumor cell suspensions from various VV-treated mice using the same treatment schedule as FIG. 5A were stained with the neopeptide 4 (Pep4, ASMTNMELM (SEQ ID NO:10))-loaded, H-2Db-labeled pentamers, anti-CD45, and anti-CD8. Data are representative of five independent experiments. (FIG. 6D) Dot plots of flow cytometry; (FIG. 6E) quantification of peptide 4-pentamer+ CD8+ T cells. Data presented as the means±SD. *P<0.05 by two-tailed Student's t-test. (FIG. 6F) Enhanced generation of neopeptide-specific memory T cells. Forty days after the virus injection, splenocytes were restimulated with neopeptide 4-loaded DCs in the presence of Golgi-plug followed by surface staining with anti-CD8 and intracellular staining with anti-107a, anti-IFN-γ, anti-IL-2, and anti-TNF-α.

FIG. 6G depicts enhanced T cell responses against various neoantigen peptides. MC38 tumor-bearing mice were intratumorally injected with PBS or various VVs as described in FIG. 6A. The splenocytes from the VV-treated tumor-bearing mice were cultured in complete RPMI1640 in 96 well plates (1×10⁵ per well) in the presence of one of indicated neoepitope peptides at various concentrations for 80 h. [3H]thymidine incorporation was measured. n=3 mice. Data presented as the means±SD. *P<0.05, **P<0.01 by two-tailed Student's t-test.

FIG. 7A-7I depict enhanced neoantigen presentation and cytolytic activity of neoantigen-specific cytotoxic T lymphocytes (CTLs). (FIG. 7A) Enhanced stimulatory potency of tumor-infiltrating DCs. Tumor-infiltrating DCs from VV-treated mice were loaded with neopeptide 4, 9, or 11, and cocultured with the neoantigens-primed T cells from mice immunized with the 11 neopeptide mixture to assess IFN-γ production; n=3 mice. Data presented as the means±SD. *P<0.05, ***P<0.001 by two-tailed Student's t-test. (FIG. 7B) Enhanced maturation of tumor-infiltrating DCs. Using a similar treatment schedule as described in FIG. 5A, cell suspensions prepared from VV-treated tumors were analyzed by flow cytometry. (FIG. 7C) Enhanced tumor infiltration of CD103+ DCs. Using the same treatment schedule as in FIG. 5A, tumor cell suspensions from VV-treated mice were analyzed by FACS; n=5 mice. Data presented as the means±SD. **P<0.01 by two-tailed Student's t-test. (FIG. 7D) Intracellular staining of IL-12 and CXCL9 of CD103+ DCs from VV-treated tumors. (FIG. 7E) qRT-PCR analysis of CXCL10 mRNA levels in CD103+ DCs isolated from VV-treated tumors; n=5 mice. Data presented as the means±SD. **P<0.01 by two-tailed Student's t-test. (FIG. 7F) Neoantigens-primed T cells proliferated more efficiently in VV-iPDL1/GM-treated mice. The neoantigens-primed T cells were labeled with 5μM CFSE and i.v. injected into VV-treated mice. Three days later, T cell proliferation was assessed by FACS. (FIG. 7G) Enhanced stimulatory effect of VV-iPDL1/GM-infected tumor cells. MC38 tumor cells infected with VVs at MOI=1 were cocultured with the neoantigens-primed T cells. IFN-γ production (left) and T cell proliferation (right) were measured. Data presented as the means±SD. *P<0.05 by two-tailed Student's t-test. (FIG. 7H) Serum of VV-iPDL1/GM-treated mice enhanced the cytolytic activity of neoantigens-primed T cells. MC38-Luc cells were cocultured with the neoantigen-specific T cells in the presence of the sera from treated MC38-bearing mice. Cytolytic activity was calculated using luciferase emission value. Data are presented as means±SD. **P<0.01 by two-tailed Student's t-test. (FIG. 7I) PD-1+ CD8+ T cells isolated from VV-treated MC38 tumors were cocultured with MC38 cells in the presence of purified iPDL1 or IgG. IFN-γ+ frequencies of PD-1+ T cells were shown from one of two independent experiments.

FIG. 7J depicts flow cytometric analysis of tumor-infiltrating cells. Using the same treatment schedule as in FIG. 5A, tumor cell suspensions from VV treated mice were analyzed by FACS. Gate strategy of CD4+ or CD8+ lymphocytes, and CD103+ DCs.

FIGS. 7K and 7L depict altered gene expression of VV-iPDL1/GM-infected MC38 tumor cells. (FIG. 7K) RNA-seq analysis of VV-iPDL1/GM-infected MC38 cells. MC38 cells were infected with VV-iPDL1/GM for the indicated times. The infected tumor cells were harvested and the extracted total RNA was used for RNA-Seq. (FIG. 7L) qRT-PCR of 11 identified genes. The gene expression level was quantified and normalized to the GADPH control. Bars depict SD (n=3 independent samples).

FIG. 8 depicts the DNA sequence of mouse version iPDL1 fusion gene (SEQ ID NO:1) and the amino acid sequence of mouse version iPDL1 (SEQ ID NO:2). The DNA stop codon, TGA, indicated by *, is a nonsense codon, which does not code for an amino acid.

FIG. 9A-9D depict the generation and characterization of a recombinant oncolytic vaccinia virus coexpressing human PD-L1 inhibitor and GM-CSF (VV-ihPDL1/GM). (FIG. 9A). A schematic diagram of a recombinant vaccinia virus shuttle vector that coexpresses soluble human PD-1 and Fc fusion protein (hPD1Fc or ihPDL1) and human GM-CSF (VV-ihPDL1/GM) and control vectors. vTK: vaccinia virus thymidine kinase gene; R: right flank sequence, L: left flank sequence, and RFP: Red Fluorescent Protein. (FIGS. 9B, 9C) Expression and secretion of ihPDL1 and GM-CSF from infected tumor cells in vitro. H226 cells were infected with the indicated VVs at an MOI of 2. 48 h later, culture media were harvested. FIG. 9B. Clarified culture media were analyzed by Western Blot using anti-human PD-1 (left gel) or anti-human IgG (right gel). Lane 1, VV-RFP culture media; lane 2, VV-GM culture media; lane 3, VV-ihPDL1/GM culture media with reducing buffer; lane 4, VV-ihPDL1/GM culture media with non-reducing buffer. FIG. 9C. Clarified culture media were also analyzed by Western Blot using anti-GM-CSF (FIG. 9C). Lane 1, VV-RFP culture media; lane 2, VV-GM culture media; lane 3, VV-ihPDL1/GM culture media. (FIG. 9D) Secretion of human PD-L1 inhibitor and GM-CSF from VV-infected tumor cells. Human tumor cells (PANC1, A375 or U87) were infected with VV-RFP, or VV-ihPDL1/GM at a MOI of 2. After 24, 48, or 72 hours, supernatants were collected and ihPDL1 (hPD1Fc) concentration was measured via ELISA. The experiments were triplicated with similar results.

FIGS. 10A and 10B depict VV-ihPDL1/GM retains the ability to preferentially replicate and kill human tumor cells. (FIG. 10A) VV-ihPDL1/GM preferentially replicates in human tumor cells. 2×10⁵ normal cells or tumor cells as indicated were infected with VV-RFP, VV-GM, or VV-ihPDL1/GM at a low dosage (MOI=0.5) for 24 h, 48 h, 72 h. Infected cells were harvested, and frozen/thawed three times to release viral particles in 1 mL media. The viral particles were titrated as described in material and methods. Experiment was repeated twice. (FIG. 10B) Oncolytic activity of VV-ihPDL1/GM against various types of human tumor cells. Human tumor cell lines (Panc1, U87, A375, or H226) were infected with the indicated VVs at a MOI of 5 or 1 for 24, 48, 72, and 96 hrs. MTT assay were performed to determine viability of different infected tumor cells. The cell survival percentage is expressed as the viability of different viral-infected cells relative to that of mock-infected cells at the time point. Data are presented as means±SD. Experiments were repeated twice.

FIG. 11A-11F depict the characterization of secreted GM-CSF and ihPDL1 from VV-ihPDL1/GM-infected tumor cells. (FIG. 11A) Supernatants of VV-infected tumor cells support TF1 cell growth. PANC1 cells were infected with VV-RFP, VV-GM or VV-ihPDL1/GM at an MOI=1 for 48 h. Supernatant was collected and filtered through a 0.22-um inorganic membrane filter (millipore, Billerica, Mass. SLGP033RB). A volume of 0.1, or 1 μL of the filtered supernatant was applied onto TF1 cells in 96-well plate. Commercial GM-CSF (2 ng/ml) was applied as a positive cell control and PBS applied as a negative control. After culturing for 48 h, MTT assay was used to evaluate proliferation of TF1 cells under different conditions. The OD value measured at 570nm corresponds with biological activity of the secreted GM-CSF. The experiment was repeated twice. p<0.001, VV-GM or VV-ihPDL1/GM vs. VV-RFP for the volume of 1 μL. (FIG. 11B) Supernatants of viral-infected tumor cells enhance CD11c+ DC differentiation. Monocytes derived from healthy PBMCs were cultured in complete RPMI-1640 media supplemented with 50 ng/mL GM-CSF and 100 ng/mL IL-4 for 3 days. All the non-adherent or loosely adherent cells were collected and resuspended in complete RPMI-1640 media supplemented with 100 ng/mL IL-4 and then aliquoted into a 12-well tissue plate. 1 μL, 10 μL, or 100 μL filter (0.1 μm)-treated supernatant of PANC1 cells infected with the indicated viruses was added to the culture wells in the 12-well plate. The culture wells added with 50 ng/mL commercial GM-CSF served for positive controls. The culture wells added with PBS served for negative controls. All the cells were incubated for another 48 h and then collected for CD11c staining and FACS. The experiment was repeated twice. (FIGS. 11C, 11D) Purified ihPDL1 binds to PD-L1⁺ tumor cells. PD-L1-transduced 293T or K562 cells (FIG. 11C) or IFN-γ pre-stimulated H226 or U251 tumor cells (FIG. 11D) were incubated with PBS (left panels), 50 μg/mL purified ihPDL1 or IgG control (sigma, St Louis, Mo.) for 30 min on ice. Cells were washed twice and followed by staining of viability and anti-PD-L1 or anti-IgG-Fc (Biolegend, San Diego, Calif.). The stained cells were analyzed by flow cytometry. Experiment was repeated twice. (FIG. 11E) ihPDL1 in the supernatants of VV-ihPDL1/GM-infected tumor cells binds to PD-L1⁺ cells. The supernatants of VV-infected PANC1 cell were concentrated using Amicon Ultra-15 Centrifugal Filter Units. PD-L1-transduced 293T, K562 cells, or IFN-γ pre-stimulated PD-L1⁺ H226 or U251 tumor cells (upper panel) were incubated with 50 μL concentrated supernatants for 30 min on ice. Cells were washed twice and followed by staining of viability and anti-IgG-Fc (Biolegend, San Diego, Calif.). Commercial hPD1Fc fusion protein, and IgG were used as positive and negative control. The stained cells were analyzed via FACS. Experiment was repeated twice. (FIG. 11F) ihPDL1 in supernatants of VV-infected tumor cells inhibits the binding of anti-PD-L1 antibody to PD-L1. The IFN-γ-stimulated U251, H226, PANC1, or A375 were incubated with 50 μL concentrated supernatants (used in FIG. 3E) for 30 min on ice. Cells were washed twice, and followed by staining of viability and anti-PD-L1 antibody (Biolegend, San Diego, Calif.). The stained cells were analyzed via FACS. All the experiments were repeated twice.

FIG. 12A-12D depict ihPDL1 secreted from infected tumor cells inhibits PD-1/PD-L1 interaction and has ADCC activity. (FIG. 12A) Surface plasmon resonance (SRP)-binding assay comparing binding affinity of purified ihPDL1 or commercial anti-PD-L1 to PD-L1. (FIG. 12B) Purified ihPDL1 from supernatants of viral-infected tumor cells inhibits PD-1/PD-L1 interaction. 1 μg/well commercial PD-L1 protein was used to coat a 96-well ELISA plate. 10 ng PD1-biotin mixed with MOCK, IgG, purified ihPDL1, or commercial anti-hPD-L1 at the indicated concentrations in a volume of 50 μL was added into the coated plate. The plate was incubated at RT for 2 h followed by extensive washing. The plate was added with diluted streptavidin-HRP and incubated at RT for 1 hr with slow shaking. After 5-time washes, 100 μL TMB HRP substrate was added and 100 μL 1N sulfuric acid was added to stop reaction when blue color is developed in the positive control wells. OD value at 450nm was measured. The inhibition activity (%)=(OD450 of Mock−OD450 of sample)/(OD450 of Mock−OD450 of background)×100%. p<0.05, ihPDL1 vs. IgG at 0.1 μg/mL; p<0.01 at 1 μg/mL and 10 μg/mL. (FIG. 12C) Purified ihPDL1 enhances T cell function in a Mix Lymphocyte Reaction assay (MLR). T-cells were isolated from healthy PBMCs and co-cultured with irradiated (2500 rads) allogeneic mature DCs at a ratio of 10:1 in the presence of MOCK, isotype IgG, purified ihPDL1, or commercial anti-PD-L1 at the indicated concentrations for 5 days. IFN-γ level in the media was measured via ELISA. Data are presented as means±SD. p<0.01, ihPDL1 vs. IgG at each indicated dosages. The experiments were triplicated with similar results. (FIG. 12D) ADCC activity of secreted ihPDL1. Serial dilutions of purified ihPDL1 or IgG control were incubated with Jurkat effector cells (Promega ADCC Bioassay Effector cells) in the presence of target cells K262/PD-L1 or IFN-γ-stimulated U251 (U251/PD-L1) tumor cells at 37° C. for 6 h, ihPDL1-mediated ADCC activity was quantified by measuring luciferase production. Data are presented as means±SD. p<0.005, ihPDL1 vs. IgG at 1000 ng/mL. The experiments were triplicated with similar results.

FIG. 13A-13C depict mouse tumor model studies. (FIG. 13A) High levels of serum ihPDL1 and GM-CSF in tumor-bearing mice treated with VV-ihPDL1/GM. H226 cells were s.c. inoculated into one side flank of NSG mice. When the median tumor volume reached 100 mm³, groups of tumor-bearing mice were injected intratumorally with 1×10⁸ pfu of VV-RFP, VV-GM, VV-ihPDL1/GM. Prior to the viral injection and 48 h post-viral injection, mice were bled for measuring serum ihPDL1 and GM-CSF levels via ELISA. Data are presented as means±SD. p<0.005, or VV-ihPDL1/GM vs. VV-RFP for ihPDL1; p<0.01, VV-GM or VV-ihPDL1/GM vs. VV-RFP for GM-CSF. (FIG. 13B) VV-ihPDL1/GM-treated mouse sera enhanced T cell activity in MLR assay. T-cells isolated from healthy PBMCs were co-cultured with irradiated allogeneic mature DCs at a ratio of 10:1 for 5 days in the presence of 100 μL different virus treated mouse sera in a volume of 200 μL. IFN-γ level in the media was measured via ELISA. Data are presented as means±SD. p<0.05, or VV-ihPDL1/GM vs. VV-RFP at the post-injection. The experiment was triplicated with similar results. (FIG. 13C) VV-ihPDL1/GM-treated mouse sear inhibited PD-1/PD-L1 interaction. PD1-biotin (10 ng) was mixed with 100 μL of different virus treated mouse sera in a volume of 200 μL. The mixture was then added into PD-L1-coated 96-well plate. After incubation at RT for 2 h, diluted streptavidin-HRP was added followed by addition with TMB substrate. The inhibition activity was expressed as (OD450 of MOCK−OD450 of sera)/(OD450 of MOCK−OD450 of background)×100%. Data are presented as means±SD. p<0.01, or VV-ihPDL1/GM vs. VV-RFP at the post-injection. The experiment was triplicated with similar results.

FIG. 14A-14B depict VV-ihPDL1/GM-treated mouse sera enhanced cytotoxicity of CAR-T cells against PD-L1⁺ tumor cells. (FIG. 14A) Mesothelin (MSLN)-targeted CAR-T cells or (FIG. 14B) CD19-targeted CAR-T cells were co-cultured with H226 tumor cells transduced with MSLN and PD-L1 (E:T=10:1) or Raji cells (E:T=5:1) in the presence of 25 μL different VV-treated mouse sera for 48 h. Killing activity of CART cells against target tumor cells was measured by luc-based CTL assay (Promega). p<0.001, VV-ihPDL1/GM-treated mouse sera vs. VV-RFP-treated mouse sera. Experiments were repeated twice.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “about” or “approximately,” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as ±10% of the value modified by the term “about.”

An “oncolytic virus” is a virus that exhibits increased replication in, and lysis of, cancer cells relative to comparable non-cancer cells; see, for example, Bartlett D L et al., 2013, Molecular Cancer 12:103-120; Kaufman H L et al., 2015, Nature Reviews Drug Discovery 14:642-662 and Chiocca E A and Rabkin S D, 2014, Cancer Immunol Res; 2; 295-300. In certain embodiments, the oncolytic virus exhibits selective replication in cancer cells and less or essentially no replication in non-cancer cells. In certain embodiments, less replication means that replication in cancer cells versus comparable non-cancer cells is at least about 30 percent greater, or at least about 50 percent greater, or at least about 80 percent greater.

Non-limiting examples of oncolytic viruses, suitable for preparing for the recombinant oncolytic viruses disclosed herein, in addition to vaccinia virus, include types of (i) adenovirus (“Ad”), for example hTERT-Ad; (ii) herpes simplex virus (“HSV”), for example G207, HSV-1716, T-VEC, and HSV-2 APK mutant; (iii) poxvirus, for example vaccinia virus, for example vSP and vvDD (tk−/vgf−) (and see below); (iv) arbovirus; (v) paramyxovirus, for example, measles virus, mumps and Newcastle disease virus; (vi) rhabdovirus, for example, vesicular stomatitis virus; (vii) picornavirus, for example Coxsackie virus, Seneca Valley Virus, and polio virus; (viii) reovirus; (ix) parvovirus; and (x) recombinant/engineered versions of any one of the above. In certain embodiments, the oncolytic virus is an oncolytic virus that has been approved by the Food and Drug Administration (FDA) or is undergoing clinical trials.

In certain embodiments, the oncolytic virus is a vaccinia virus. In certain non-limiting embodiments, the oncolytic virus is an engineered (also referred to as “recombinant”) vaccinia virus. In certain non-limiting embodiments, the virus is a recombinant vaccinia virus based on the Western Reserve (“WR”) strain of vaccinia, for example, the WR strain commercially available from the American Type Culture Collection as ATCC No. VR1354. Other vaccinia virus strains suitable for engineering include, but are not limited, to the Wyeth strain (ATCC VR-1536), the Lederle-Chorioallantoic strain (ATCC VR-325), and the CL strain (ATCC VR-117). In certain non-limiting embodiments, the oncolytic virus is an engineered vvDD vaccinia viral construct comprising, for example, a modified version of a virus described in U.S. Pat. Nos. 7,208,313, 8,506,947, and United States Patent Application Publications Nos. 2003/0031681 and 2007/0154458, McCart et al., 2001, Cancer Research 61:8751-8757 and/or Thorne Set al., 2007, J. Clin. Invest. 117:3350-3358, all of which are incorporated by reference herein in their entries. For example, but not by way of limitation, the vaccinia virus can have deletions of the thymidine kinase (tk) and/or vaccinia growth factor (vgf) genes.

“Subject” or “individual” or “patient” refers to any subject, particularly a mammalian subject, for whom diagnosis, prognosis, or therapy is desired. In some embodiments, the subject has cancer. In some embodiments, the subject had cancer at some point in the subject's lifetime. In various embodiments, the subject's cancer is in remission, is re-current or is non-recurrent. The subject may be human or animal. The mammal can be a human, non-human primate, mouse, rat, dog, cat, horse, or cow, but are not limited to these examples.

“IgG-Fc fusion proteins,” “Fc fusion proteins,” “Fc chimeric fusion proteins,” “Ig-based Chimeric Fusion proteins,” or “Fc-tag proteins” are composed of the Fc domain of IgG genetically linked to a peptide or protein of interest. In various implementations of the recombinant oncolytic virus disclosed herein, IgG-Fc fusion proteins are used as an inhibitor to the binding partners of the peptide or protein with which the IgG-Fc is fused. For example, an inhibitor of PD-L1 can be an IgG-Fc fused with PD-1, e.g., an IgG1-Fc fused with human PD-1, or an IgG1-Fc fused with mouse PD-1; and an inhibitor of PD-1 can be an IgG-Fc fused with PD-L1.

“Treat”, “treating”, and “treatment”, etc., refer to any action providing a benefit to a patient. In various aspects, “treat”, “treating”, and “treatment” refer to an action providing a benefit to a patient at risk for development of tumor or tumor metastasis, or having a cancer or a tumor, or detected with cancerous cells, including improvement in the condition through lessening or suppression of primary tumor size or presence or size of distant tumor, prevention or delay in progression of the disease, prevention or delay in the onset of disease states or conditions which occur secondary to cancers. Treatment, as used herein, encompasses both prophylactic and therapeutic treatment. The term “prophylactic” when used, means to reduce the likelihood of a reoccurrence, reduce the severity of a reoccurrence, or reduce an occurrence, within the context of the treatment of cancer.

“Chimeric antigen receptor” or “CAR” or “CARs” refers to engineered receptors, which graft an antigen specificity onto cells (for example natural killer (NK) cells, T cells such as naïve T cells, central memory T cells, effector memory T cells or combination thereof). CARs are usually composed of an antigen-specific targeting domain, a transmembrane domain, an intracellular signaling domain, an extracellular spacer domain, and a co-stimulatory domain.

A “cancer” or “tumor” refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastatses. Examples of cancer include, but are not limited to B-cell lymphomas (Hodgkin's lymphomas and/or non-Hodgkins lymphomas), brain tumor, breast cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, brain cancer, and prostate cancer, including but not limited to androgen-dependent prostate cancer and androgen-independent prostate cancer.

“Antibody” as used herein refers to polyclonal antibodies, monoclonal antibodies, humanized antibodies, single-chain antibodies, and fragments thereof such as Fab, F(ab′)₂, Fv, and other fragments which retain the antigen binding function of the parent antibody. In an embodiment, the antibody specifically binds Hsp90α as described herein. The antibody may be polyclonal antibodies, monoclonal antibodies, humanized antibodies, single-chain antibodies, and fragments thereof such as Fab, F(ab′)2, Fv, and other fragments which retain the sialidase activity of the parent antibody. The antibody may be a recombinant antibody. The term “recombinant human antibody” can include a human antibody produced using recombinant DNA technology.

Individual tumors with numerous genetic mutations can contain high numbers of potentially immunogenic neoantigens, also referred to as tumor neoantigens herein. Despite the presence of immunogenic neoantigen epitopes in each tumor, spontaneous priming and activation of neoantigen-specific T cells are inefficient in the majority of cancer patients.

Herein we generate an engineered oncolytic vaccinia virus ((VV)-iPDL1/GM) that coexpresses a PD-L1 inhibitor and granulocyte-macrophage colony-stimulating factor (GM-CSF). The engineered oncolytic virus is capable of activating neoantigen-specific T cell responses by the likely co-action of viral replication, GM-CSF stimulation, and PD-L1 inhibition on tumor cells and immune cells, providing a new oncolytic immunotherapy. Oncolytic viruses possess the potential to offer a facile in situ vaccination approach to activate T cell responses by locoregional immune activation, immunogenic oncolytic tumor cell death, mutant neoantigen release and presentation, and alteration of the immunosuppressive tumor microenvironment. Recent clinical trials demonstrated that oncolytic virotherapy with talimogene laherparepvec (T-Vec) promoted intratumoral T cell infiltration and improved anti-PD-1 or cytotoxic T-lymphocyte associated protein (CTLA) immunotherapy. However, how oncolytic viruses activate tumor neoantigen-specific T cell responses is still poorly studied. Moreover, it remains an unmet medical need to solve the problem that the reactive upregulation of PD-L1 expression in the tumor microenvironment after virus administration can cause tumor resistance to oncolytic immunotherapy.

Oncolytic virus coexpressing GM-CSF and an inhibitor of PD-1/PD-L1 interaction (VV-iPDL1/GM) has been demonstrated capable of producing the inhibitor of PD-1/PD-L1 interaction, which systematically binds to PD-L1+ tumor cells and immune cells. The intratumoral injections with VV-iPDL1/GM produced iPDL1, enhanced neoantigen presentation, and activated systemic T cell responses against dominant, as well as subdominant neoantigens, resulting in the effective rejection of both virus-injected and distant tumors. Thus, this double-armed oncolytic virus is capable of activating neoantigen-specific T cell responses by the likely co-action of PD-L1 inhibition on tumor cells and immune cells, viral replication, and GM-CSF stimulation.

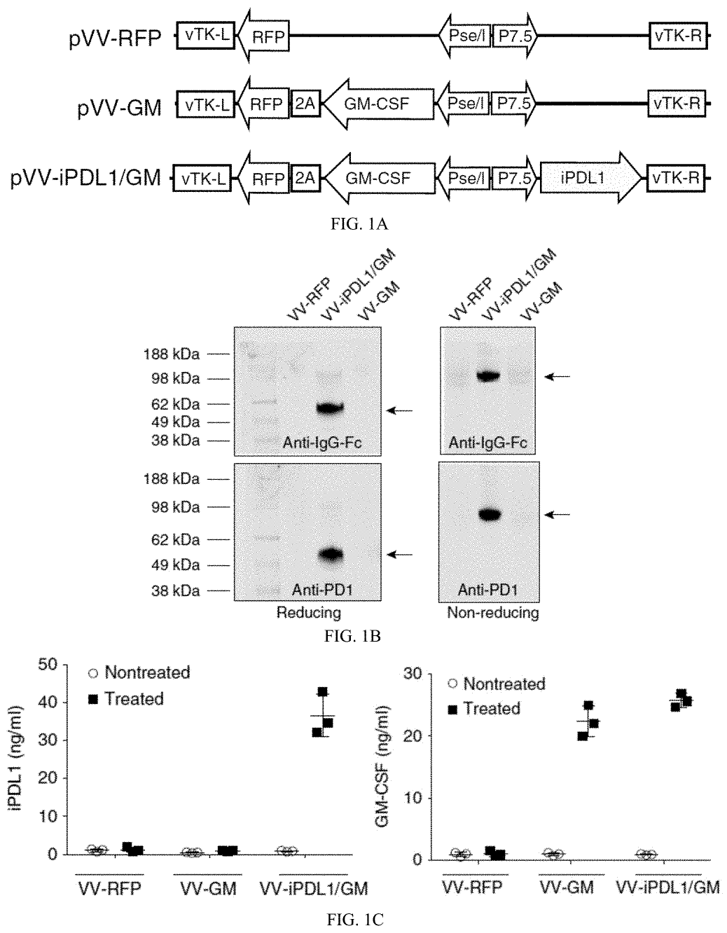

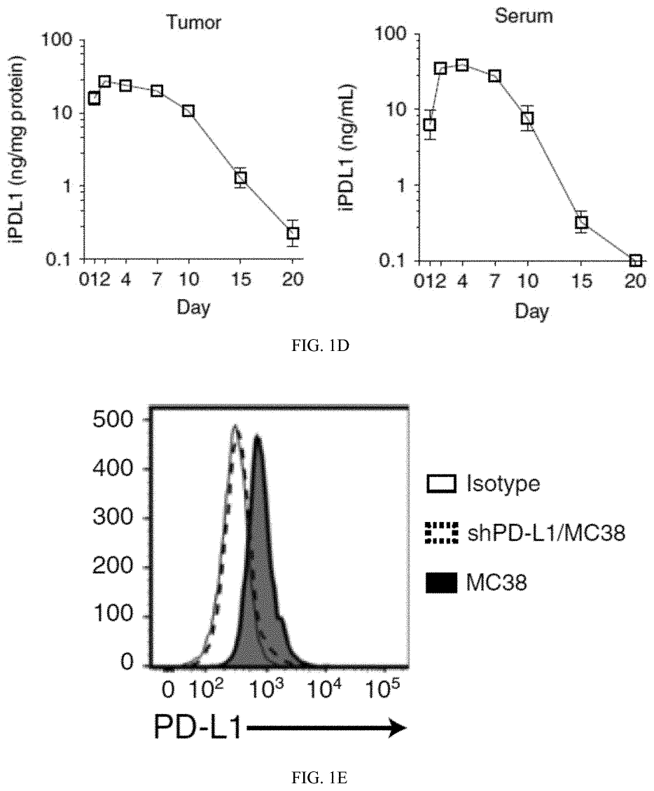

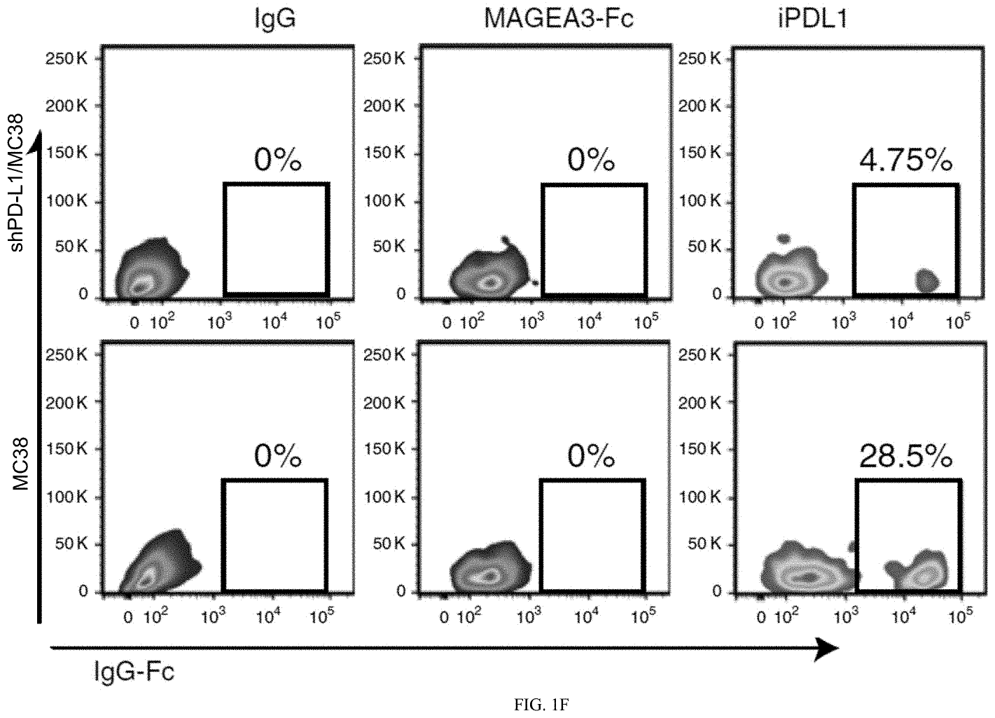

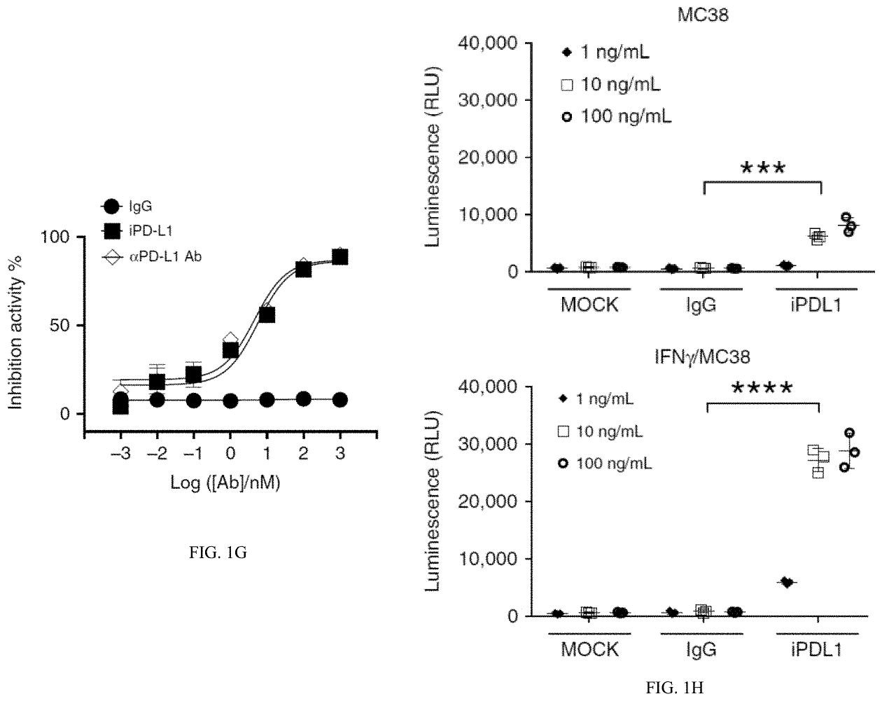

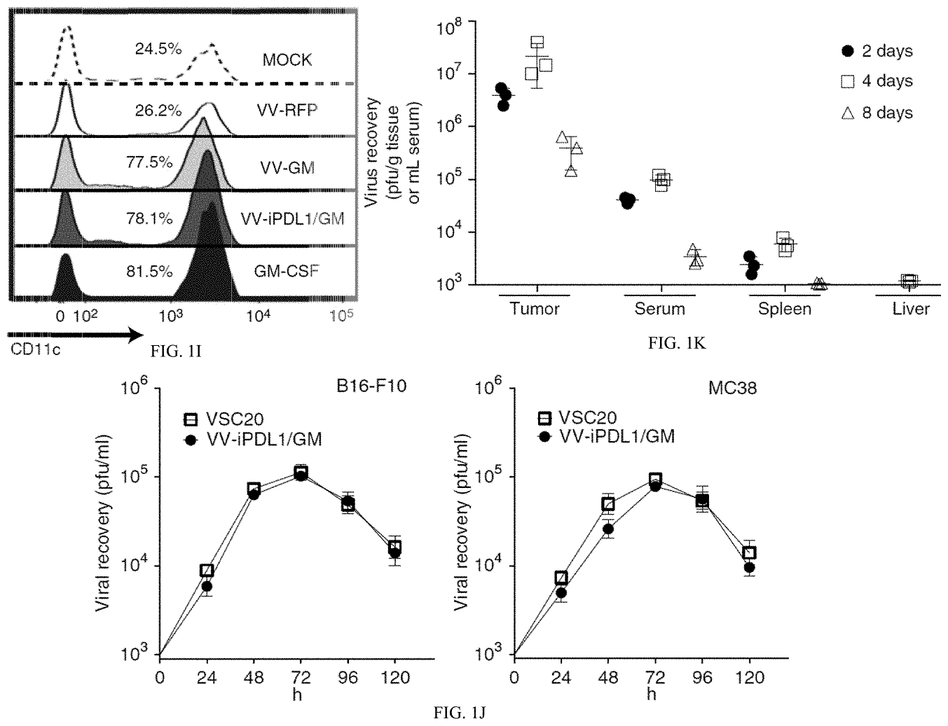

Some embodiments provide for a recombinant oncolytic virus, which is an oncolytic virus introduced with one or more nucleic acid sequences encoding (1) a polypeptide inhibitor of PD-L1 or a polypeptide inhibitor of PD-1, and (2) GM-CSF. Upon infecting and replicating in neoplastic cells, the recombinant oncolytic virus causes infected cells to secrete polypeptide inhibitor of PD-L1 (or polypeptide inhibitor of PD-1) and the GM-CSF, and in further embodiments, ultimately lysis of the infected cells (as shown in FIG. 1M).

In various aspects, the secreted polypeptide inhibitor of PD-L1 is able to bind to PD-L1+ immune cells, such as bone marrow-derived dendritic cells or T cells. In further aspects, the secreted polypeptide inhibitor of PD-L1 and GM-CSF, or tumor cells infected with the recombinant oncolytic virus so as to secrete the polypeptide inhibitor of PD-L1 and GM-CSF, are able to enhance T cell response to one or more neoantigens (or to tumors bound to said neoantigens), such as Pep1-Pep11 as shown by SEQ ID NOs:3-24, and to PD-L1+ tumor cells, wherein the enhanced T cell response includes increased identification of the neoantigens, increased IFN-γ secretion, and/or increased tumor filtration by the T cells compared to that induced by anti-PD-L1 antibody. In additional aspects, tumor cells infected with the recombinant oncolytic virus have an increased transcription or expression level of one or more of genes, including programmed cell death 1 (PDCDI), programmed cell death 1 ligand 1 (PDCD1L), calreticulin (CALR), CD74 (CD74), heat shock protein family A (Hsp70) member 1B (HSPA1B), heat shock protein family A (Hsp70) member 5 (HSPA5), protein disulfide isomerase family A member 4 (PDIA4), protein disulfide isomerase family A member 5 (PDIA5), colony stimulating factor 2 (CSF2), Fos proto-oncogene AP-1 transcription factor subunit (FOS), and mitogen-activated protein kinase kinase 7 (MAP2K7), relative to tumor cells un-infected with the oncolytic virus or infected with oncolytic virus without nucleic acids encoding the GM-CSF and the polypeptide inhibitor of PD-L1.

Further embodiments provide for a combination of two or more different oncolytic viruses, wherein a first oncolytic virus is introduced with a nucleic acid encoding a polypeptide inhibitor of PD-L1 or a polypeptide inhibitor of PD-1, and a second oncolytic virus is introduced with a nucleic acid encoding GM-CSF. In some implementations, the combination of two or more different oncolytic viruses is administered to a subject having a tumor or in need of cancer prophylaxis or treatment.

In additional embodiments, an oncolytic virus which is engineered with or introduced with a nucleic acid encoding a polypeptide inhibitor of PD-L1 or a polypeptide inhibitor of PD-1, suitable for administration to a subject having a tumor or in need of cancer prophylaxis or treatment. Another oncolytic virus introduced with a nucleic acid encoding GM-CSF is also provided, suitable for administration to a subject having a tumor or in need of cancer prophylaxis or treatment. In some implementations, the first oncolytic virus containing a nucleic acid encoding a polypeptide inhibitor of PD-L1 or a polypeptide inhibitor of PD-1 is administered to a subject in need thereof concurrently, or sequentially, with the second oncolytic virus containing a nucleic acid encoding GM-CSF.

In some embodiments, a recombinant oncolytic virus includes one or more nucleic acid sequences encoding (1) a polypeptide inhibitor of human PD-L1, and (2) human GM-CSF. In some embodiments, the recombinant oncolytic virus includes (1) a first nucleic acid sequence that encodes a polypeptide inhibitor of human PD-L1, and (2) a second nucleic acid sequence that encodes human GM-CSF. In some embodiments, the recombinant oncolytic virus includes (1) a first nucleic acid sequence of SEQ ID NO:51, which encodes a polypeptide inhibitor of human PD-L1, and (2) a second nucleic acid sequence of SEQ ID NO:53, which encodes human GM-CSF. In some embodiments, the recombinant oncolytic virus including one or more nucleic acid sequences encoding a polypeptide inhibitor of human PD-L1 and encoding human GM-CSF, upon infecting and replicating in a neoplastic cell, produces (1) a first polypeptide having an amino acid sequence of SEQ ID NO:52, which is inhibitor of human PD-L1, i.e., binds human PD-L1 (or binds PD-L1+ cell) and inhibits the interaction of human PD-L1 and PD-1, and (2) a second polypeptide having an amino acid sequence of SEQ ID NO:54, which is human GM-CSF.

DNA sequence encoding soluble human PD-1
(extracellular portion) (in lower case) - IgG1 Fc
(in upper case):
(SEQ ID NO: 51)
5′-Atgcagatcccacaggcgccctggccagtcgtctgggcggtgctaca
actgggctggcggccaggatggttcttagactccccagacaggccctgga
acccccccaccttctccccagccctgctcgtggtgaccgaaggggacaac
gccaccttcacctgcagcttctccaacacatcggagagettcgtgctaaa
ctggtaccgcatgagccccagcaaccagacggacaagctggccgccttcc
ccgaggaccgcagccagcccggccaggactgccgcttccgtgtcacacaa
ctgcccaacgggcgtgacttccacatgagcgtggtcagggcccggcgcaa
tgacagcggcacctacctctgtggggccatctccctggcccccaaggcgc
agatcaaagagagcctgcgggcagagctcagggtgacagagagaagggca
gaagtgcccacagcccacGACAAAACTCACACATGCCCACCGTGCCCAGC
ACCTGAACTCCTGGGGGGACCGTCAGTCTTCCTCTTCCCCCCAAAACCCA
AGGACACCCTCATGATCTCCCGGACCCCTGAGGTCACATGCGTGGTGGTG
GACGTGAGCCACGAAGACCCTGAGGTCAAGTTCAACTGGTACGTGGACGG
CGTGGAGGTGCATAATGCCAAGACAAAGCCGCGGGAGGAGCAGTACAACA
GCACGTACCGTGTGGTCAGCGTCCTCACCGTCCTGCACCAGGACTGGCTG
AATGGCAAGGAGTACAAGTGCAAGGTCTCCAACAAAGCCCTCCCAGCCCC
CATCGAGAAAACCATCTCCAAAGCCAAAGGGCAGCCCCGAGAACCACAGG
TGTACACCCTGCCCCCATCCCGGGAGGAGATGACCAAGAACCAGGTCAGC
CTGACCTGCCTGGTCAAAGGCTTCTATCCCAGCGACATCGCCGTGGAGTG
GGAGAGCAATGGGCAGCCGGAGAACAACTACAAGACCACGCCTCCCGTGC
TGGACTCCGACGGCTCCTTCTTCCTCTACAGCAAGCTCACCGTGGACAAG
AGCAGGTGGCAGCAGGGGAACGTCTTCTCATGCTCCGTGATGCACGAGGC
TCTGCACAACCACTACACGCAGAAGAGCCTCTCCCTGTCTCCGGGTAAAT
GA.
Soluble human PD-1 (extracellular) (in italics) -
IgG1 Fc amino acid sequence:
(SEQ ID NO: 52)
MQIPQAPWPVVWAVTQLGWRPGWELDSPDRPWNPPIESPALLVVTEGDNA
TFTCSFSNTSESEVLNWYKIISPSNQTDKLAAFPEDRSQPGQDCRFRVTQ
LPNGRDEHMSVVRARRNDSGTYLCGAISLAPKAQIKESTRAELRVTERRA
EVPTAHDKTHTCPPCPAPELLGGPSVFLEPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDK
SRWQQGNVFSCSVMHEALHNHYTQKSLSLSPGK.
DNA sequence encoding human GM-CSF:
(SEQ ID NO: 53)
5′-ATGTGGCTGCAGAGCCTGCTGCTCTTGGGCACTGTGGCCTGCAGCAT
CTCTGCACCCGCCCGCTCGCCCAGCCCCAGCACGCAGCCCTGGGAGCATG
TGAATGCCATCCAGGAGGCCCGGCGTCTCCTGAACCTGAGTAGAGACACT
GCTGCTGAGATGAATGAAACAGTAGAAGTCATCTCAGAAATGTTTGACCT
CCAGGAGCCGACCTGCCTACAGACCCGCCTGGAGCTGTACAAGCAGGGCC
TGCGGGGCAGCCTCACCAAGCTCAAGGGCCCCTTGACCATGATGGCCAGC
CACTACAAGCAGCACTGCCCTCCAACCCCGGAAACTTCCTGTGCAACCCA
GATTATCACCTTTGAAAGTTTCAAAGAGAACCTGAAGGACTTTCTGCTTG
TCATCCCCTTTGACTGCTGGGAGCCAGTCCAGGAGTGA.
Human GM-CSF amino acid sequence:
(SEQ ID NO: 54)
MWLQSLLLLGTVACSISAPARSPSPSTQPWEHVNAIQEARRLLNLSRDTA
AEMNETVEVISEMFDLQEPTCLQTRLELYKQGLRGSLTKLKGPLTMMASH
YKQHCPPTPETSCATQIITFESFKENLKDFLLVIPFDCWEPVQE.

In some embodiments, the recombinant oncolytic virus includes one or more nucleic acid sequences encoding (1) a polypeptide inhibitor of mouse PD-L1, and (2) mouse GM-CSF. In some embodiments, the recombinant oncolytic virus includes (1) a first nucleic acid sequence that encodes a polypeptide inhibitor of mouse PD-L1, and (2) a second nucleic acid sequence that encodes mouse GM-CSF. In some embodiments, the recombinant oncolytic virus includes (1) a first nucleic acid sequence of SEQ ID NO:1, which encodes a polypeptide inhibitor of mouse PD-L1, and (2) a second nucleic acid sequence encoding mouse GM-CSF. In some embodiments, the recombinant oncolytic virus including one or more nucleic acid sequences encoding a polypeptide inhibitor of mouse PD-L1 and encoding mouse GM-CSF, upon infecting and replicating in a neoplastic cell, produces (1) a first polypeptide having an amino acid sequence of SEQ ID NO:2, which is inhibitor of mouse PD-L1, i.e., binds mouse PD-L1 (or binds PD-L1+ cell) and inhibits the interaction of mouse PD-L1 and PD-1, and (2) a second polypeptide, which is mouse GM-CSF.

In various implementations, the recombinant oncolytic virus is a vaccinia virus with a transgene. In further implementations, the recombinant oncolytic virus is a combined thymidine kinase-deleted (TK−) and vaccinia growth factor-deleted (VGF−) vaccinia virus, with a transgene. In certain embodiments, the oncolytic virus is a recombinant vaccinia virus with an inactivating mutation of its thymidine kinase gene, vaccinia growth factor gene, or both. In certain non-limiting embodiments, a vaccinia virus has an inactivating mutation in one or more gene where the product of said gene or genes functions in viral replication. For example, one or more of the following genes can bear an inactivating mutation: the gene encoding the ribonucleotide reductase-large subunit, the gene encoding the ribonucleotide reductase-small subunit, the gene encoding thymidylate kinase, the gene encoding DNA ligase, the gene encoding dUTPase, the tk gene, and the vaccinia virus growth factor (vgf) gene. In certain embodiments, an inactivating mutation is a mutation that either reduces or eliminates activity of the gene product. In certain embodiments, gene activation can be achieved by mutagenesis, e.g., site-directed mutagenesis or PCR-mediated mutagenesis. Alternatively or additionally, in certain embodiments, a nucleic acid can be inserted into one or more of the foregoing genes to achieve inactivation. In certain non-limiting embodiments, a nucleic acid encoding a protein can be inserted into one or more of the foregoing genes to achieve inactivation and to further achieve expression of the nucleic acid.

In certain embodiments, the oncolytic virus is a herpes simplex virus. In certain embodiments, the oncolytic virus is an adenovirus. In certain embodiments, the oncolytic virus is a reovirus, a vesicular stomatitis virus, a poliovirus, a senecavirus, or a Semliki Forest virus. Additional strains of oncolytic virus, preferably configurable for oncolytic virus therapy, are described in U.S. Pat. No. 7,208,313, US20100272686, US20200000862 (also WO2018145033), US20200208122 (also WO2017118865) and U.S. Pat. No. 9,862,932, which are incorporated by reference in their entireties herein.

In various implementations, inhibitors of PD-L1, of PD-L2 or of PD-1 are polypeptides that bind PD-L1, PD-L2, or PD-1, respectively, and block them from interacting with their cognate binding partners. In various implementations, an inhibitor of PD-L1, or an inhibitor of PD-L2, is an immunoglobulin Fc fusion protein, in which an immunoglobulin Fc region is fused with PD-1 (or an extracellular domain of PD-1); and an inhibitor of PD-1 is an IgG-Fc fusion protein, in which an IgG-Fc is fused with PD-L1 or PD-L2. In some embodiments, the inhibitor of PD-1/PD-L1 interaction is a polypeptide inhibitor of PD-L1, (denoted as “iPDL1”), which binds to PD-L1 and blocks it from interacting with PD-1. In further embodiments, the inhibitor of PD-L1 is a fusion protein comprising soluble PD-1 extracellular domain and the crystallizable fragment of the immunoglobulin class G (IgG-Fc).

Another polypeptide inhibitor of PD-L1 is durvalumab, a human monoclonal antibody against PD-L1, or an antigen-binding fragment of durvalumab. Further examples of inhibitors of PD-L1, of PD-L2, or of PD-1, including but not limited to IgG-Fc fusion proteins, are described in US20170189476, U.S. Pat. No. 8,008,449, WO2006/121168, U.S. Pat. No. 8,354,509, WO2009/114335, U.S. Pat. No. 7,943,743 and U.S. Patent Application Publication No. 20120039906, which are incorporated by reference in their entireties. One or more nucleic acid sequences encoding these antibodies or antibody fragments are provided as embodiments of part of the compositions disclosed herein.

Recombinant oncolytic viruses (or replicating virus) can be prepared by various techniques to introduce one or more “template nucleic acids,” and the template nucleic acids encode a polypeptide inhibitor of PD-L1, a polypeptide inhibitor of PD-1, and/or GM-CSF. In some implementations, a template nucleic acid (a “transgene”) is inserted into a locus in the genetic material of a vaccinia virus, or another oncolytic virus, by homologous recombination. In some implementations, the vaccinia virus is a combined thymidine kinase-deleted (TK−) and vaccinia growth factor-deleted (VGF−) vaccinia virus. Deletion of either the TK gene or VGF genes can significantly decrease pathogenicity compared with wild type virus. In some implementations, an oncolytic virus is a thymidine kinase gene-inactivated oncolytic vaccinia virus. In some implementations, VSC20, a vaccinia virus that has the lacZ gene inserted into its VGF sites (therefore VGF-depleted virus), is used as the background virus, and a vaccinia shuttle plasmid is created and used to allow for homologous recombination of the template nucleic acid(s) into the TK locus of VSC20, so as to create double deleted vaccinia virus with additions of the template nucleic acid(s). For example, a vector containing the template nucleic acid(s) is digested with restriction enzymes SalI and SpeI, and the segment containing the template nucleic acid(s) is ligated into the SalI and SpeI sites of shuttle plasmid, which places the template nucleic acid(s) under the control of the vaccinia synthetic early/late promoter; and it is flanked by portions of the vaccinia TK gene, which allows for homologous recombination into this locus in the background virus. In various implementations, the template nucleic acid(s) is placed in vaccinia virus under the control of one or more early/late promoters, such as Pse/1, Pse/2, P7.5 early/late, P7.5 early, P28 late, P11 late. In some implementations, the template nucleic acid encoding a polypeptide inhibitor of PD-L1 or a polypeptide inhibitor of PD-1 and the template nucleic acid encoding GM-CSF are operably linked to the Pse/1 promoter and the P7.5 early/late promoter.

Various embodiments provide for a cell infected with a recombinant oncolytic virus, wherein the recombinant oncolytic virus comprises one or more nucleic acid sequences encoding a polypeptide inhibitor of PD-L1 or a polypeptide inhibitor of PD-1, and encoding GM-CSF, wherein the cell secretes at least the polypeptide inhibitor encoded by the one or more nucleic acid sequences of the recombinant oncolytic virus. In various aspects, the infected cell is a mammalian cell. In some aspects, the infected cell is a tumor cell. In some aspects, the infected cell secretes both the polypeptide inhibitor encoded by the one or more nucleic acid and the GM-CSF.

Various embodiments provide for a system, or a combination, comprising a recombinant oncolytic virus and a mammalian cell, wherein the recombinant oncolytic virus contains one or more nucleic acid sequences encoding a polypeptide inhibitor of PD-L1 or a polypeptide inhibitor of PD-1 and GM-CSF, and upon infecting the mammalian cell by the recombinant oncolytic virus, the mammalian cell secretes the polypeptide inhibitor and the GM-CSF.

A further embodiment provides for a system, comprising (1) a recombinant oncolytic virus, (2) one or more tumor cells, and (3) an immune cell.

In various aspects of the system, the recombinant oncolytic virus infects at least one of the one or more tumor cells, and upon infection by the recombinant oncolytic virus, the at least one infected tumor cell secretes the polypeptide inhibitor and the GM-CSF, inducing infiltration or activating the immune cell. In some aspects of the system, the recombinant oncolytic virus contains one or more polynucleotides encoding at least a polypeptide inhibitor of PD-L1.

In some aspects of the system, the recombinant oncolytic virus contains one or more polynucleotides encoding at least a polypeptide inhibitor of PD-L1; and upon infection by the recombinant oncolytic virus, the at least one infected tumor cell secretes the polypeptide inhibitor of PD-L1, inducing infiltration of or activating the immune cell, wherein the immune cell is positive or expresses PD-L1. In further aspects, the secreted polypeptide inhibitor of PD-L1 also binds to PD-L1 on another tumor cell in the system. In some aspects, the immune cell is a T cell, a CD8+ T cell, a Treg cell, a dendritic cell, a myeloid-derived suppressor cell, or a CD45+ hematopoietic cell.

Various embodiments provide for serum or plasma obtained from mammals administered with the recombinant oncolytic virus disclosed herein, which can be used to activate neoantigen-specific T cell responses in a subject in need thereof.

In some implementations, serum or plasma is obtained from a mammal 2, 3, 4, 5, 6, or 7 days after the mammal receives an administration of the recombinant oncolytic virus. In some implementations, obtained serum or plasma is assayed for, or detected with, presence of the polypeptide inhibitor and/or the GM-CSF, which are encoded by the one or more nucleic acids that are engineered in the oncolytic virus.

Various embodiments provide for a cell culture medium, or a supernatant, collected from a mammalian cell culture, wherein the mammalian cells are infected with a recombinant oncolytic virus, wherein the recombinant oncolytic comprises one or more nucleic acid sequences encoding a polypeptide inhibitor of PD-L1 or a polypeptide inhibitor of PD-1 and encoding GM-CSF, wherein the collected cell culture medium, or the supernatant, contains the polypeptide inhibitor of PD-L1 or the polypeptide inhibitor of PD-1. In some aspects, the infected mammalian cell is a tumor cell.

In some implementations, a recombinant oncolytic virus enters a mammalian cell or a target cell via endocytosis, thereby infecting the mammalian cell or target cell. In other implementations, a recombinant oncolytic virus may contain a specific receptor to enter host cells, e.g., adenoviruses (Ads) are able to bind coxsackie and adenovirus receptor (CAR), integrins, or cluster of differentiation 46 (CD46); and herpes simplex virus (HSV) uses nectin or herpesvirus entry mediator.

Various embodiments also provide for one or more vectors encoding a polypeptide inhibitor of PD-L1, a polypeptide inhibitor of PD-1, and/or GM-CSF. In some embodiments, a first vector contains a nucleic acid sequence encoding a polypeptide inhibitor of PD-L1, or another vector contains a nucleic acid sequence encoding a polypeptide inhibitor of PD-1, and a second vector contains a nucleic acid sequence encoding GM-CSF. In other embodiments, one vector contains a first nucleic acid sequence encoding a polypeptide inhibitor of PD-L1 and a second nucleic acid sequence encoding GM-CSF.

In some implementations, a vector includes a first nucleic acid sequence of SEQ ID NO:51, which encodes a polypeptide inhibitor of human PD-L1, and a second nucleic acid sequence of SEQ ID NO:53, which encodes human GM-CSF. In some implementations, a vector includes a first nucleic acid sequence of SEQ ID NO:1, which encodes a polypeptide inhibitor of mouse PD-L1, and a second nucleic acid sequence encoding mouse GM-CSF.

In further implementations, the nucleic acid sequence(s) encoding the polypeptide inhibitor and the GM-CSF is operably linked to the Pse/1 promoter and the P7.5 early/late promoter.

Various embodiments provide that a recombinant oncolytic virus disclosed herein, and/or sera obtained from a mammal administered with a recombinant oncolytic virus, is provided in a pharmaceutical composition, which also includes a pharmaceutically acceptable excipient. In certain embodiments, a recombinant oncolytic virus is administered as a therapeutic composition together with a physiologic buffer. In some embodiments, a pharmaceutical composition comprising a pharmaceutically acceptable excipient and a quantity, or unit dose, of a recombinant oncolytic virus that contains one or more nucleic acids encoding GM-CSF and a polypeptide inhibitor of PD-L1 (or a polypeptide inhibitor of PD-1). In other embodiments, a pharmaceutical composition comprising a pharmaceutically acceptable excipient and a therapeutically effective amount, or unit dose, of serum or plasma obtained from a mammal injected with the recombinant oncolytic virus. In certain embodiments, the therapeutic composition is in lyophilized form. In certain embodiments, the presently disclosed subject matter provides a syringe comprising an effective amount of the therapeutic composition. In certain embodiments, the recombinant oncolytic virus disclosed herein can be prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof.

Pharmaceutically acceptable excipient refers to an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. The pharmaceutical compositions according to the invention can also contain any pharmaceutically acceptable carrier. Pharmaceutically acceptable carrier refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits. Pharmaceutically acceptable solid or liquid carriers may be added to enhance or stabilize the composition, or to facilitate preparation of the composition. The carrier may also include a sustained release material.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration.

Further embodiments provide a pharmaceutical unit dosage composition, which comprises a recombinant oncolytic virus at about 1-2×10⁷ plaque forming units (pfu), 2-3×10⁷ pfu, 3-4×10⁷ pfu, 4-5×10⁷ pfu, 5-6×10⁷ pfu, 6-7×10⁷ pfu, 7-8×10⁷ pfu, 8-9×10⁷ pfu, 1-2×10⁸ pfu, 2-3×10⁸ pfu, 3-4×10⁸ pfu, 4-5×10⁸ pfu, 5-6×10⁸ pfu, 6-7×10⁸ pfu, 7-8×10⁸ pfu, 8-9×10⁸ pfu, 1-3×10⁹ pfu, 3-5×10⁹ pfu, or 5-9×10⁹ pfu, or 1-5×10¹⁰ pfu, wherein the recombinant oncolytic virus contains one or more nucleic acid sequences encoding (1) a polypeptide inhibitor of PD-L1 or a polypeptide inhibitor of PD-1, and (2) GM-CSF. In certain embodiments, the amount of virus administered can be between about 1×10⁷ and 1×10¹⁰ infectious viral particles or pfu, or between about 1×10⁷ and 1×10⁹ pfu/m² surface area of the subject to be treated. In certain embodiments, the virus can be administered at a dose that can comprise about 1×10⁸ pfu. In certain embodiments, the amount of virus administered can be between about 1×10³ and 1×10¹² viral particles or pfu, or between about 1×10⁵ and 1×10¹⁰ pfu, or between about 1×10⁵ and 1×10⁸ pfu, or between about 1 ×10⁸ and 1 ×10¹⁰ pfu.

Methods are provided using one or more compositions disclosed herein for treatment, mitigation, reducing the size of primary tumor, reducing metastases or distant tumor size, inhibiting or reducing the extent/severity of recurring tumors, and/or increasing immune response against various tumors or cancers, including but not limited to, adenoma, colon adenoma, melanoma, neoplasm of mammary, pancreatic cancer, glioblastoma, lung cancer, glioma, osteosarcoma, skin tumor (such as melanoma), lymphoma, brain tumor, breast cancer, prostate cancer, basal cell cancer, lung cancer, leukemia, colon cancer. In some implementations, the recombinant oncolytic virus disclosed herein is not used to treat a subject with lymphoma or lymphoma-derived tumors. In various implementations, the treatment methods are effective in the presence of CD8+ T cells, or the subject to be treated in the methods have functional CD8+ T cells.

In some embodiments of the treatment methods, a pharmaceutical unit dose of the recombinant oncolytic virus is administered to the subject in need thereof. In further aspects, a pharmaceutical unit dose of the recombinant oncolytic virus is administered to each primary tumor of the subject. In further aspects, a pharmaceutical unit dose of the recombinant oncolytic virus is administered to a tumor of the subject on each day, and the pharmaceutical unit dose is repeatedly administered for one or more times. In some implementations, the recombinant oncolytic virus is administered intratumorally. In some implementations, the recombinant oncolytic virus is administered intravenously (e.g., via IV infusion), intraperitoneally, intramuscularly, intradermally, transdermally, rectal, intraurethrally, inravaginally, intranasally, or intrathecally. The routes of administration can vary with the location and nature of the tumor.

In certain embodiments, administration of the recombinant oncolytic virus, or the sera obtained from a mammal infected with the recombinant oncolytic virus, can occur by continuous infusion over a selected period of time. In certain embodiments, a recombinant oncolytic vaccinia virus as described herein, or a pharmaceutical composition containing the same, can be administered at a therapeutically effective dose by infusion over a period of about 15 mins, about 30 mins, about 45 mins, about 50 mins, about 55 mins, about 60 minutes, about 75 mins, about 90 mins, about 100 mins, or about 120 mins or longer.

The recombinant oncolytic vaccinia virus or the pharmaceutical composition of the present disclosure can be administered as a liquid dosage, wherein the total volume of administration is about 1 ml to about 5 ml, about 5 ml to 10 ml, about 15 ml to about 20 ml, about 25 ml to about 30 ml, about 30 ml to about 50 ml, about 50 ml to about 100 ml, about 100 ml to 150 ml, about 150 ml to about 200 ml, about 200 ml to about 250 ml, about 250 ml to about 300 ml, about 300 ml to about 350 ml, about 350 ml to about 400 ml, about 400 ml to about 450 ml, about 450 ml to 500 ml, about 500 ml to 750 ml or about 750 ml to 1000 ml.

In certain embodiments, a single dose of virus can refer to the amount administered to a subject or a tumor over a 1, 2, 5, 10, 15, 20 or 24 hour period. In certain embodiments, the dose can be spread over time or by separate injection. In certain embodiments, multiple doses (e.g., 2, 3, 4, 5, 6 or more doses) of the vaccinia virus can be administered to the subject, for example, where a second treatment can occur within 1, 2, 3, 4, 5, 6, 7 days or weeks of a first treatment. In certain embodiments, multiple doses of the recombinant oncolytic virus can be administered to the subject over a period of 1, 2, 3, 4, 5, 6, 7 or more days or weeks. In certain embodiments, the recombinant oncolytic vaccinia virus or the pharmaceutical composition as described herein can be administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the recombinant oncolytic vaccinia virus or the pharmaceutical composition can be, in certain instances, once daily, twice daily, once every week, once every three weeks, once every four weeks (or once a month), once every 8 weeks (or once every 2 months), once every 12 weeks (or once every 3 months), or once every 24 weeks (once every 6 months).

In certain embodiments, the recombinant oncolytic virus can be administered in an amount sufficient to induce oncolysis (or reduction in tumor size) in at least about 20% of cells in a tumor, in at least about 30% of cells in a tumor, in at least about 40% of cells in a tumor, in at least about 50% of cells in a tumor, in at least about 60% of cells in a tumor, in at least about 70% of cells in a tumor, in at least about 80% of cells in a tumor, or in at least about 90% of cells in a tumor.

Some embodiments provide a method of treating a subject suffering from cancer, including administering to the subject an effective amount of a recombinant oncolytic virus, so as to induce infiltration of one or more T cells into the cancer, wherein the recombinant oncolytic virus contains one or more nucleic acid sequences encoding GM-CSF and a polypeptide inhibitor of PD-L1 or a polypeptide inhibitor of PD-1.

In a further implementation, a method of treating a subject suffering from cancer includes (a) administering to the subject an effective amount of the recombinant oncolytic virus to induce infiltration of one or more T cells into the cancer, resulting in tumor-infiltrated T cells; (b) isolating the tumor-infiltrated T cells from the cancer of the subject; (c) expanding the tumor-infiltrated T cells ex vivo, forming expanded tumor-infiltrated T cells; and (d) transferring the expanded tumor-infiltrated T cells to the subject suffering from cancer.

Further embodiments of the treatment methods also include administering to the subject in need thereof an additional therapeutic agent, which includes but is not limited to an inhibitor of PD-1, an inhibitor of PD-L1, and/or a chemotherapeutic agent. In some embodiments, this additional therapeutic agent (e.g., inhibitor of PD-1 and/or the inhibitor of PD-L1) is administered systematically to the subject, whereas the composition of the recombinant oncolytic virus is administered intratumorally or delivered to the tumor.

An inhibitor of PD-1 can be an anti-PD-1 antibody or a PD-1-binding fragment thereof. An inhibitor of PD-L1 can be an anti-PD-L1 antibody or a PD-L1-binding fragment thereof. Other inhibitors of PD-1, PD-L1 and/or PD-L2 are disclosed in U.S. Pat. No. 8,008,449, WO2006/121168, U.S. Pat. No. 8,354,509, WO2009/114335, U.S. Pat. No. 7,943,743 and U.S. Patent Application Publication No. 20120039906, which are incorporated by reference in their entireties.

Exemplary chemotherapeutic agents include but are not limited to Albumin-bound paclitaxel (nab-paclitaxel), Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Ipilimumab, Irinotecan, Mechlorethamine, Melphalan, Mercaptopurine, Methotrexate, Mitoxantrone, Ocrelizumab, Ofatumumab, Oxaliplatin, Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, or a combination thereof.

In certain embodiments, the subject suffering from cancer is treated with a cancer therapy before the treatment with the recombinant oncolytic virus.

Various embodiments provide for a method of enhancing cytotoxicity of immune cells engineered with a chimeric antigen receptor (CAR) against tumor cells, wherein the method comprises contacting the tumor cells with the immune cells engineered with the CAR in the presence of sera obtained from a mammal treated with a recombinant oncolytic virus, thereby enhancing the cytotoxicity against the tumor cells, wherein the recombinant oncolytic virus contains one or more nucleic acids encoding (in expressive form) GM-CSF and a polypeptide inhibitor of PD-L1, and the mammal having been treated with the recombinant oncolytic virus secrete the GM-CSF and the polypeptide inhibitor of PD-L1 into plasma. In further implementations, the tumor cells are positive or present a first antigen, and the immune cells are engineered with a CAR that contains an antigen-specific targeting domain configured to target the first antigen. In one implementation, the tumor cells are positive or present mesothelin, and the immune cells are engineered with a mesothelin-targeted CAR. In another implementation, the tumor cells are positive or present PD-L1, and the immune cells are engineered with a CD19-targeted CAR. In various implementations, the tumor cells are also PD-L1+.

Methods are provided for enhancing immune cell therapy in treating, alleviating, or reducing the severity of cancer in a subject, wherein the methods comprise (1) administering to the subject an effective amount of an immune cell engineered with a CAR that targets a tumor antigen, and (2) administering to the subject an effective amount of: a recombinant oncolytic virus disclosed herein, or serum obtained from a mammal treated with the recombinant oncolytic virus, to increase infiltration of immune cells to the tumor. In various implementations, the recombinant oncolytic virus contains one or more nucleic acids that encode a polypeptide inhibitor of PD-L1 (or a polypeptide inhibitor of PD-1), GM-CSF, or both.

Additional embodiments provide for a method for generating tumor infiltrating oncolytic-virus induced T cells, which includes (a) administering, to a subject having a cancer, an effective amount of a recombinant oncolytic virus disclosed herein to induce infiltration of one or more T cells into the cancer, resulting in tumor-infiltrated T cells; (b) isolating the tumor-infiltrated T cells from the cancer of the subject. In further implementations, the method for generating tumor infiltrating oncolytic-virus induced T cells further includes (c) expanding the tumor-infiltrated T cells ex vivo.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### 1.1 Generation and Characterization of an Armed Oncolytic Vaccinia Virus (VV) Coexpressing a PD-L1 Inhibitor and GM-CSF (VV-iPDL1/GM).

We generated an engineered oncolytic VV coexpressing a murine soluble PD-1 extracellular domain fused with IgG1 Fc as a PD-L1 inhibitor (i.e., iPDL1) and murine GM-CSF (VV-iPDL1/GM), in the backbone of a tumor-selective double-deleted oncolytic VV, in which thymidine kinase (TK) and vaccinia growth factor viral genes had been deleted (FIG. 1A). A recombinant oncolytic VV-GM expressing murine GM-CSF and a recombinant oncolytic VV-RFP expressing the marker ref fluorescent protein (RFP) were also generated and produced. High levels of both GM-CSF and iPDL1 (soluble PD-1-IgG Fc) proteins in a dimer were produced and efficiently released from VV-iPDL1/GM-infected tumor cells in vitro and in vivo, as detected by western blot and enzyme-linked immunosorbent assay (ELISA; FIGS. 1B-1D, 1K). Importantly, high levels of iPDL1 were detected in the sera of VV-iPDL1/GM-treated tumor-bearing mice for a long period of time (over 15 days) after intratumor injection (FIG. 1D).

iPDL1 protein purified from the supernatants of VV-iPDL1/GM-infected tumor cells was able to bind to PD-L1+ tumor cells, but not to PD-L1-knocked down tumor cells in vitro (FIGS. 1E, 1F). In addition, it was shown that MAGEA3-IgG Fc fusion proteins failed to bind to PD-L1+ tumor cells, further ruling out the non-specific binding of IgG Fc domain to tumor cells. iPDL1 had a comparable IC₅₀ value with the commercial anti-PD-L1 antibody in blocking PD-1/PD-L1 interaction, shown in Table 1, as manifested by a competitive ELISA assay (FIG. 1G).

TABLE 1
IC₅₀ value.

| | IgG | iPD-L1 | αPD-L1 Ab |
|---|---|---|---|
| IC₅₀ (nM) | No inhibition | 5.63 | 4.53 |

It was found that iPDL1, but not IgG1 Fc, efficiently mediated antibody-dependent cell-mediated cytotoxicity (ADCC) against IFNγ-treated, PD-L1-expressing tumor cells (FIG. 1H). Supernatants derived from VV-GM- or VV-iPDL1/GM-infected MC38 tumor cells also had GM-CSF functionality in driving bone marrow (BM)-derived monocytes to differentiate into CD11c⁺ DCs (FIG. 1I). Moreover, the insertion of iPDL1 gene into the oncolytic VV did not interfere with the infection and replication of VV-iPDL1/GM in vitro and in vivo (FIGS. 1J, 1K, 1M). Taken together, these data demonstrate that the armed oncolytic virus VV-iPDL1/GM can infect tumor cells to produce and secrete high levels of functional iPDL1 and GM-CSF proteins.

1.2. PD-L1 Inhibitors Secreted from VV-iPDL1/GM-Infected Cells Bind to Upregulated PD-L1 on Tumor Cells and Immune Cells in Autocrine and Paracrine Manners.

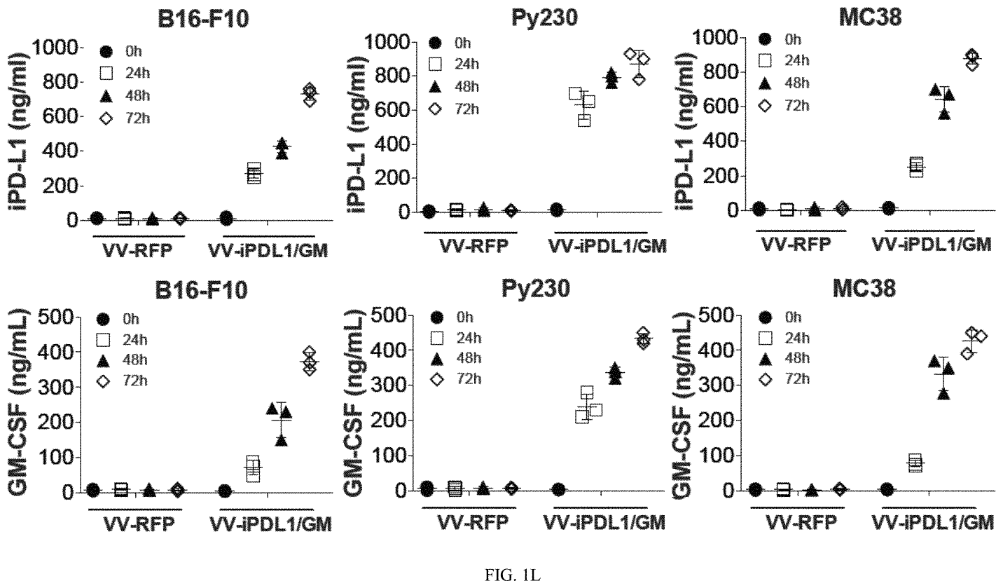

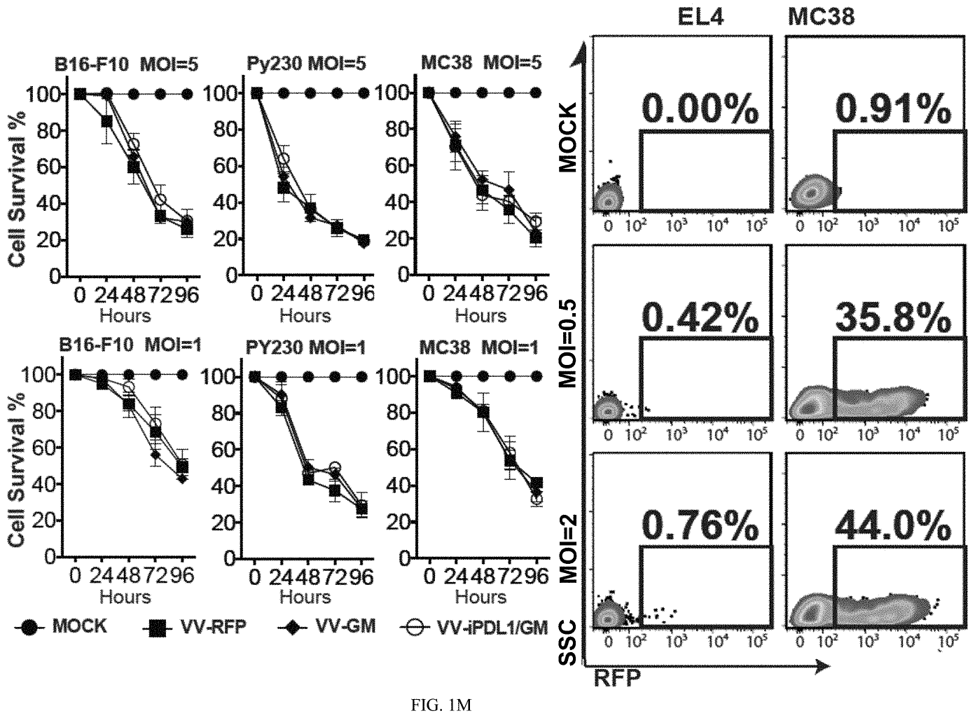

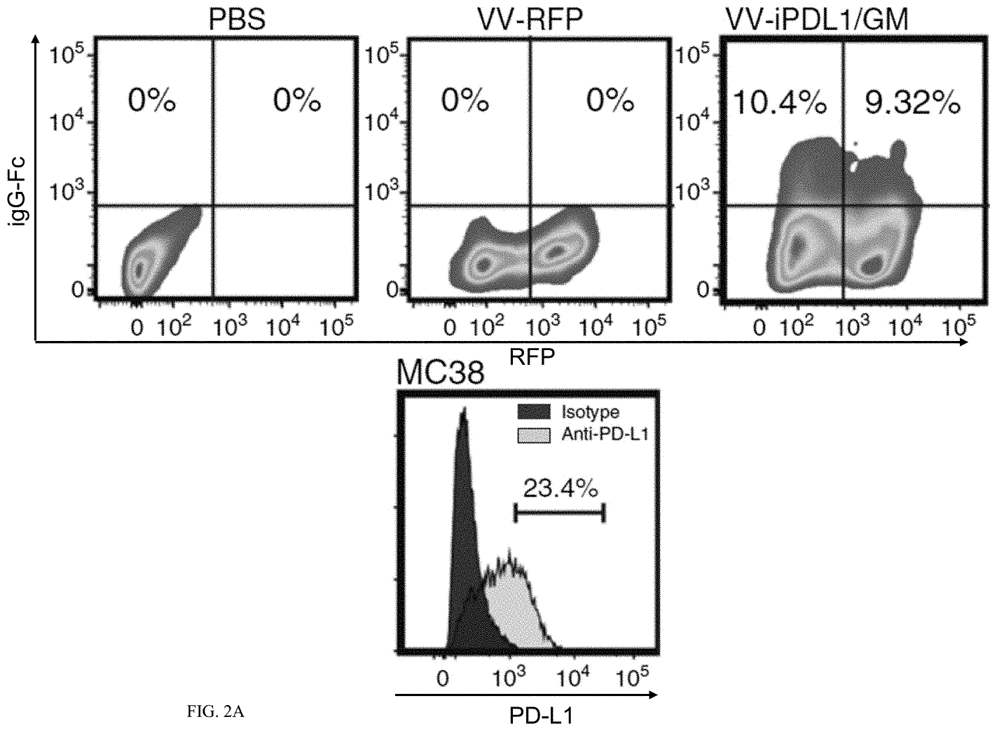

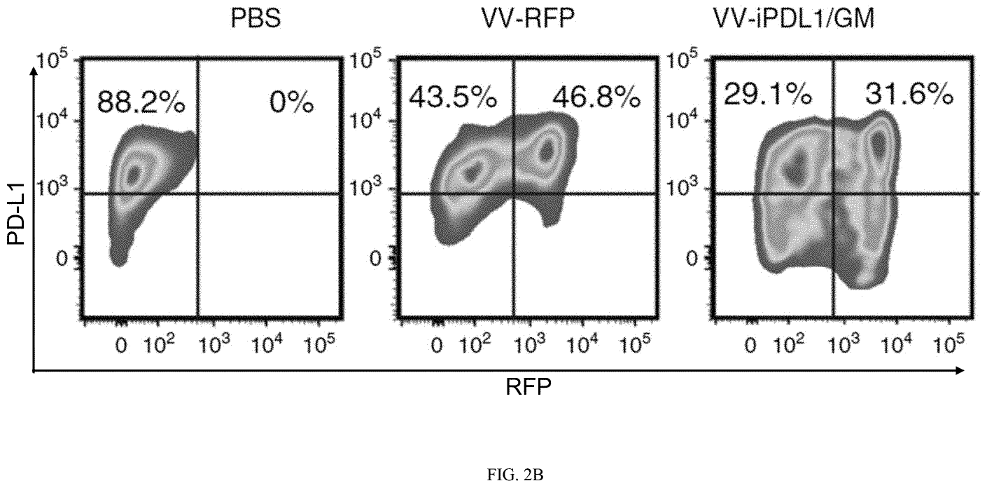

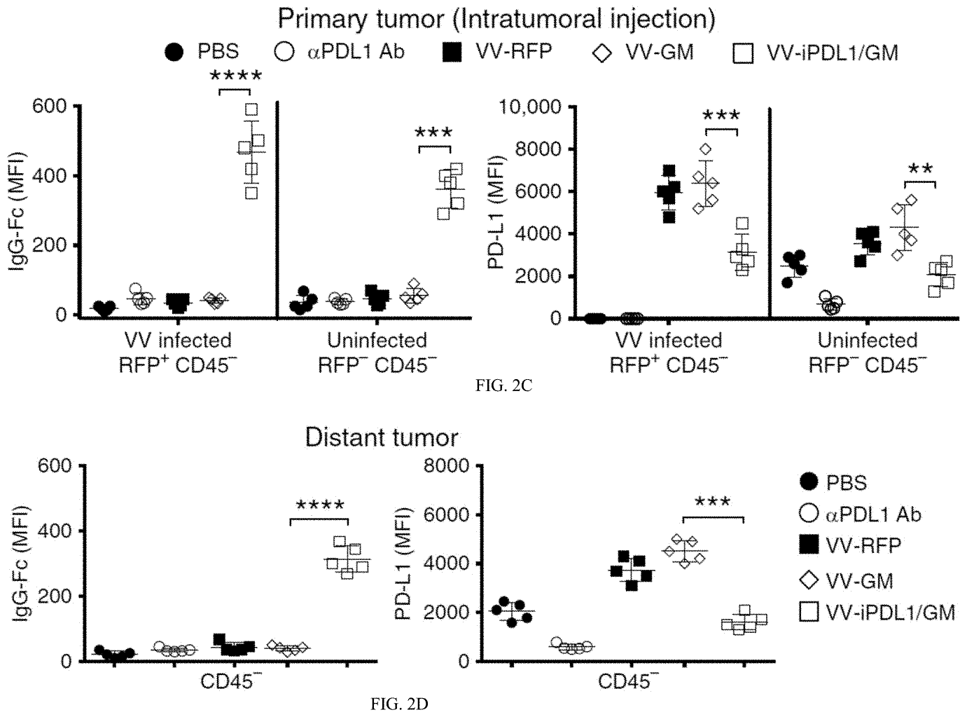

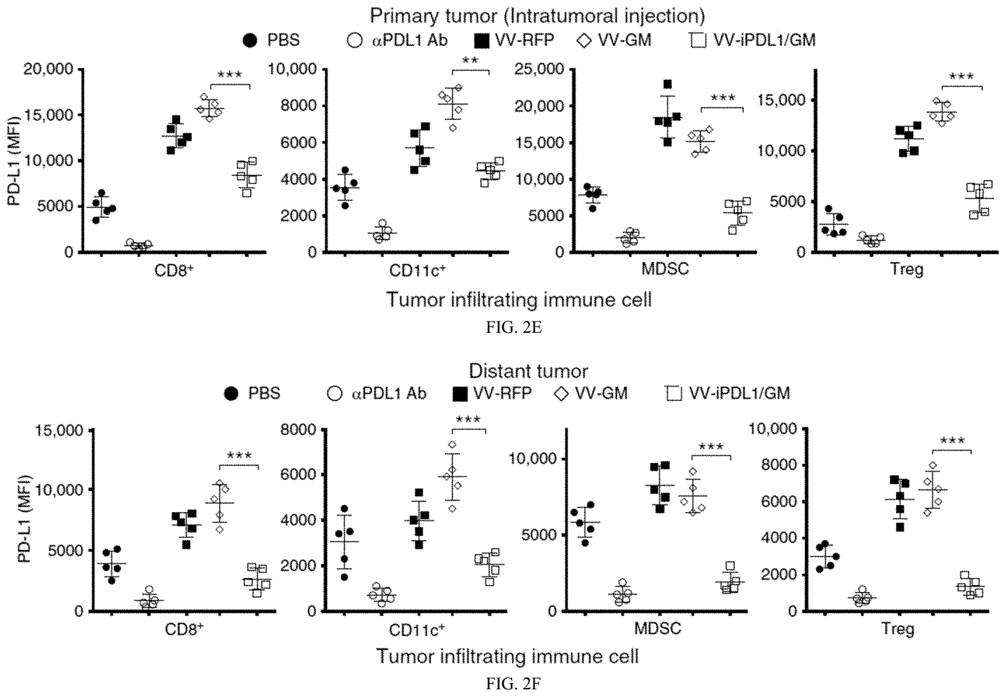

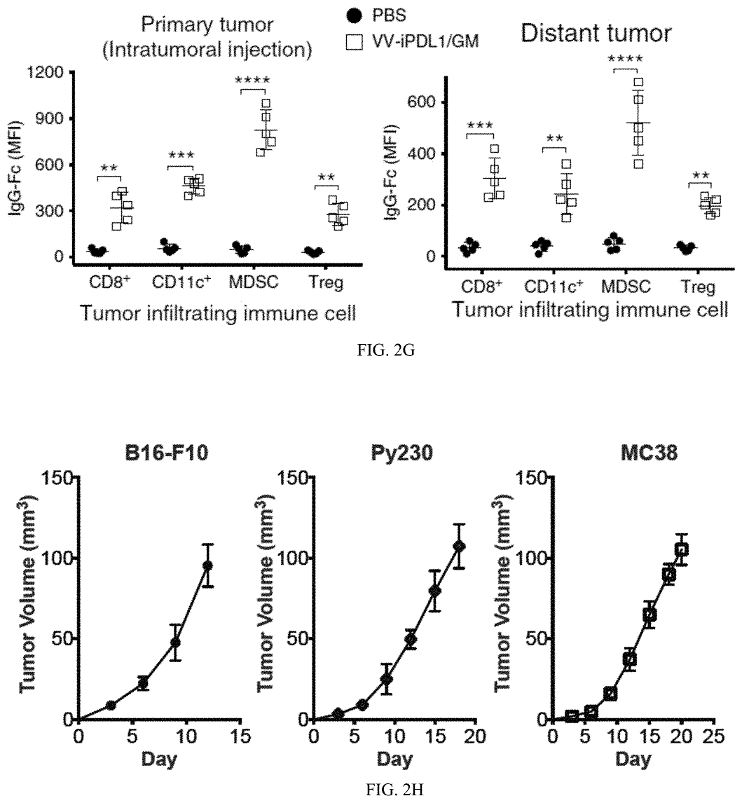

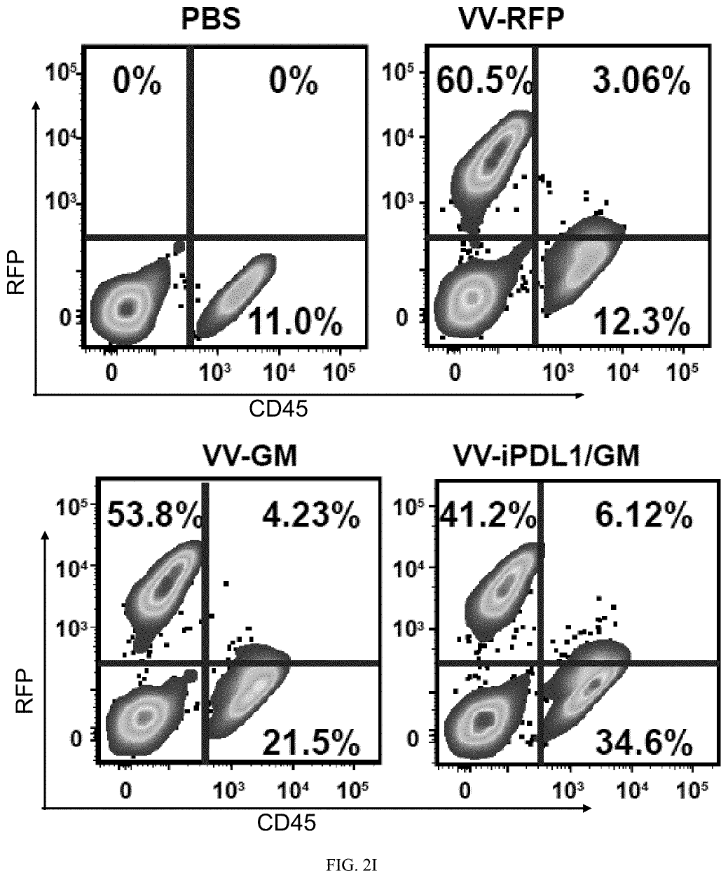

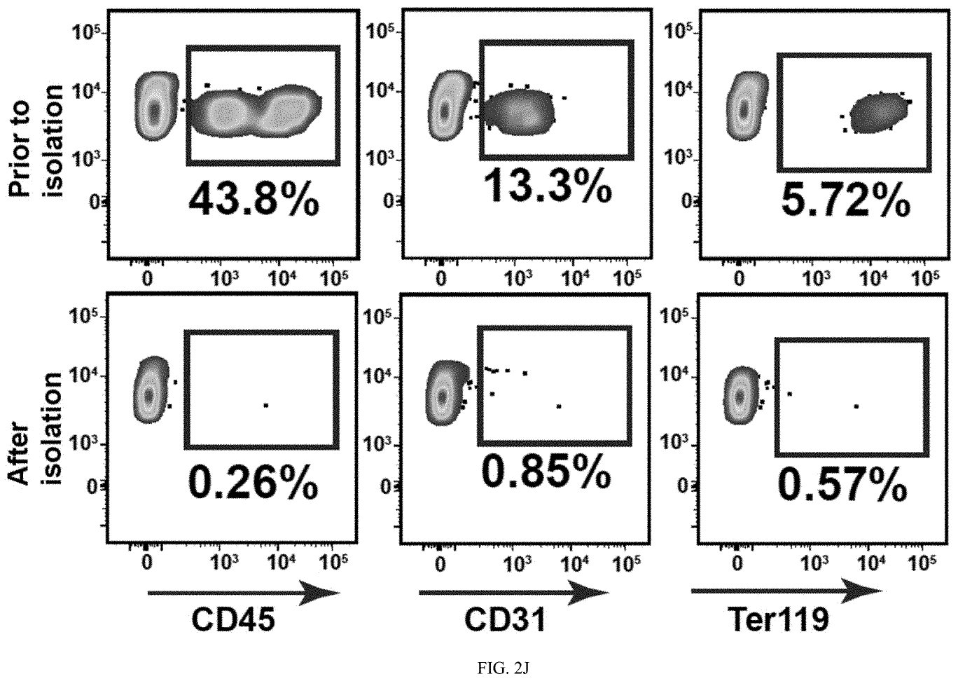

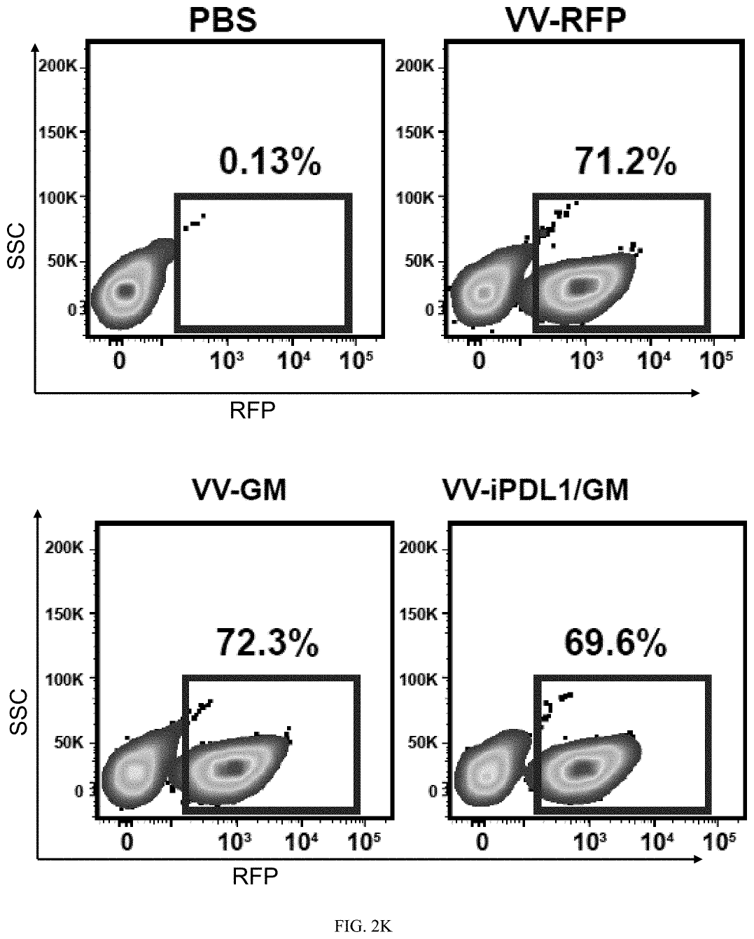

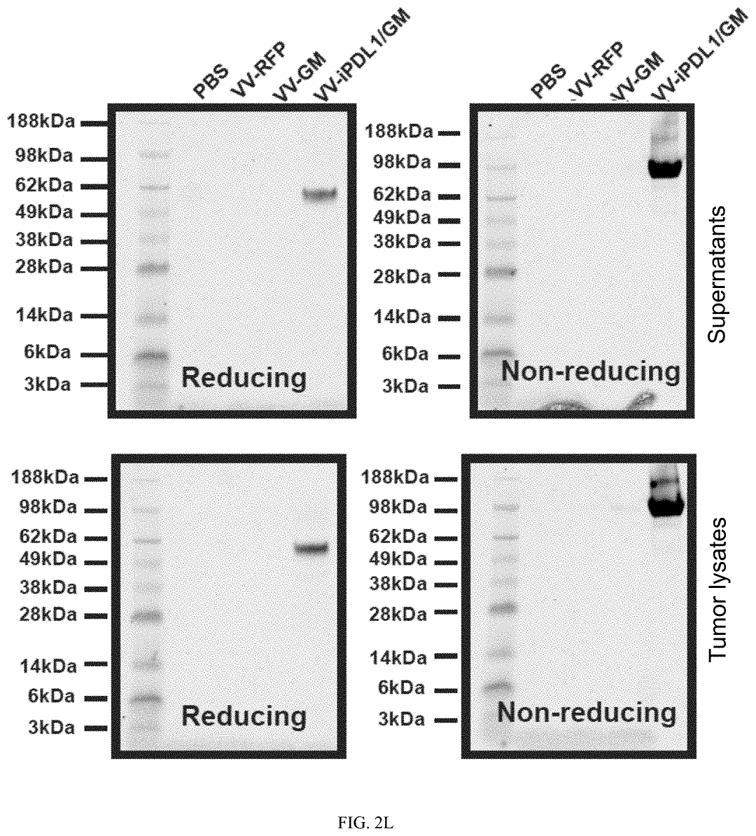

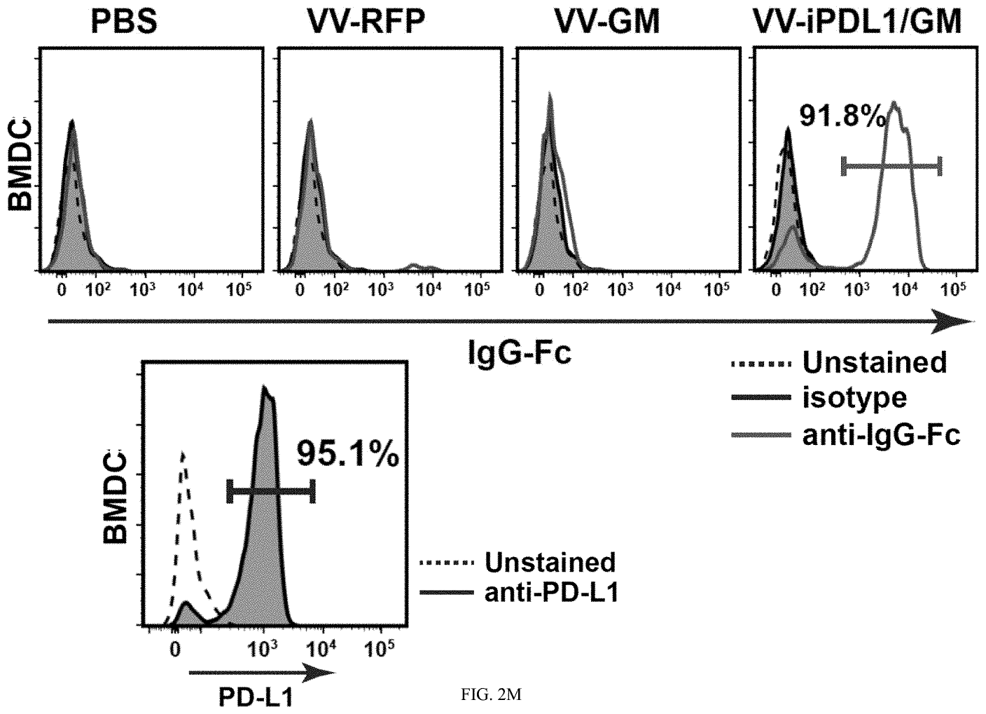

We examined whether iPDL1 secreted from VV-iPDL1/GM-infected tumor cells was able to bind PD-L 1 on tumor cells in cell culture. FIG. 2A shows that the secreted iPDL1 (PD-1-IgG Fc) bound to PD-L1 on the virus-infected (RFP positive) tumor cells, as well as uninfected (RFP negative) tumor cells in autocrine and paracrine manners by flow cytometry staining with an anti-IgG Fc to detect the binding of secreted iPDL1 to PD-L1 on tumor cells. The percentage of PD-L1+ VV-iPDL1/GM-infected (RFP+) or uninfected (RFP−) tumor cells was significantly lower than that of VV-RFP-infected (RFP+) or uninfected (RFP−) tumor cells (FIG. 2B), indicating that the binding of the iPDL1 secreted from VV-iPDL1/GM-infected cells to PD-L1 on tumor cells partially blocked PD-L1 staining with an PD-L1 antibody.

We then examined whether VV-iPDL1/GM-infected cells were able to secrete iPDL1 that can bind PD-L1 on tumor cells in vivo. Groups of mice bearing MC38 tumors in right and left flanks were injected with the recombinant VV-iPDL1/GM, VV-GM, or VV-RFP only into the tumors in the left flank. Single-cell suspensions prepared from treated primary tumors or untreated, distant tumors were analyzed by flow cytometry staining with an anti-IgG Fc. Consistent with the observations in recent studies, intratumoral injections with oncolytic viruses (VV-RFP) also significantly upregulated PD-L1 expression on both VV-RFP-infected (RFP+) and uninfected (RFP−) CD45− non-leukocyte cells, including tumor and stromal cells, compared to PD-L1 expression on PBS-treated tumors (FIG. 2C). Lower levels of PD-L1 expression in the tumors injected with VV-iPDL1/GM, compared to VV-RFP, indicating that the secreted iPDL1 bound to PD-L1 (FIG. 2C). Indeed, the binding of secreted iPDL1 (PD-1-IgG Fc) to PD-L1 on VV-iPDL1/GM-treated CD45− non-leukocyte cells was detected (FIG. 2C). Importantly, iPDL1 (PD-1-IgG Fc) secreted from treated primary tumors also bound to PD-L1 on CD45− cells in untreated, distant tumors (FIG. 2D). These data indicate that the secreted iPDL1 binds to PD-L1 on CD45− tumor and stromal cells in VV-iPDL1/GM-treated primary and untreated, distant tumors in autocrine and paracrine manners.

We further examined whether the iPDL1 secreted from VV-iPDL1/GM-treated tumors was able to bind PD-L1 on immune cells in vivo. The upregulation of PD-L1 expression on CD45+ hematopoietic cell infiltrates, including DCs, MDSCs, and T cells, was observed in both VV-RFP-treated and untreated tumors, compared to PD-L1 expression in PBS-treated tumors (FIGS. 2E, 2F). Lower levels of PD-L1 expression on CD45+ hematopoietic cell infiltrates in VV-iPDL1/GM-injected tumors and distant tumors were detected compared to the PD-L1 expression in VV-RFP or VV-GM-injected tumors and distant tumors (FIGS. 2E, 2F). FIG. 2G shows the binding of secreted iPDL1 to PD-L1 on immune cells from VV-iPDL1/GM-treated and untreated distant tumors. Furthermore, we investigated the infection and secretion of iPDL1 after intratumor injections of VV-iPDL1/GM in tumor-bearing mice (FIG. 2H). FIGS. 2I, 2J and 2K show the efficient infection (RFP+) of tumor cells (CD45−CD31−Ter119−) by VV-iPDL1/GM in vivo. FIGS. 2L and 2M show the secretion of the iPDL1 dimer from the isolated tumor cells after intratumor injections with the binding activity to PD-L1 on immune cells. Taken together, these data demonstrate that iPDL1 secreted from VV-iPDL1/GM-treated tumors is able to systemically bind to PD-L1 on tumor cells and immune cells in vivo.

### 1.3. Enhanced Antitumor Activities Against Primary and Distant Tumors.

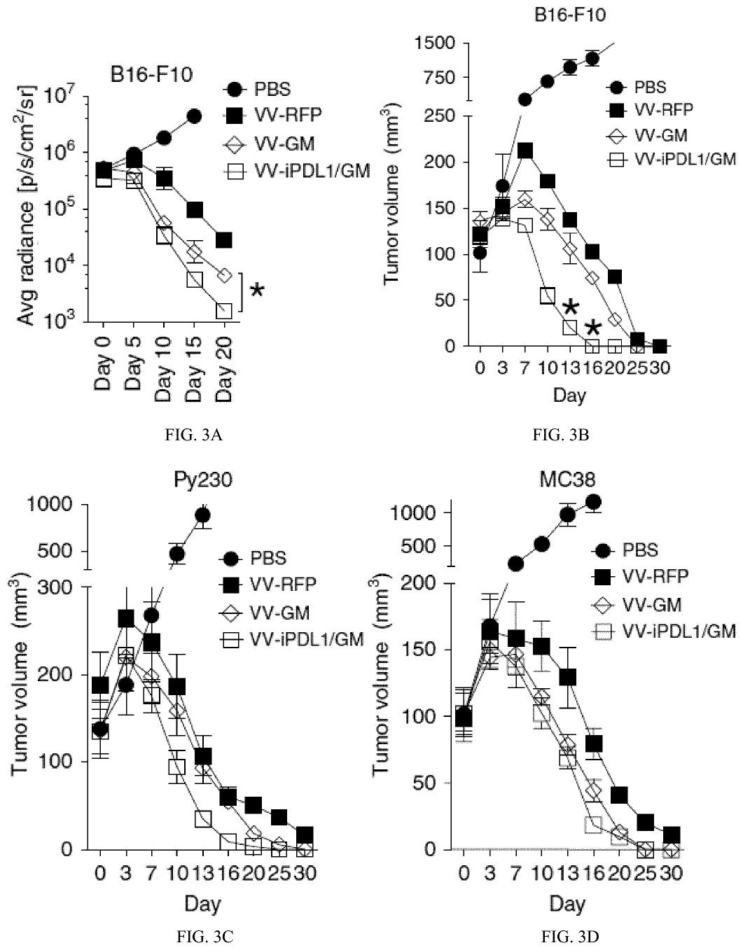

We evaluated the antitumor activity of VV-iPDL1/GM using a luciferase+ B16-F10 melanoma syngeneic transplant mouse model, which was weakly immunogenic. Tumor-bearing mice received intratumoral injections of various VVs or PBS as described. Although intratumoral injections with VV-RFP or VV-GM drastically inhibited tumor growth, both bioluminescence monitoring (FIG. 3A) and caliper measurement (FIG. 3B) showed that VV-iPDL1/GM was more potent in inhibiting B16-F10 tumor growth. Intratumoral injections of the recombinant VVs also drastically inhibited the growth of Py230 breast cancer and MC38 colon adenocarcinoma (FIGS. 3C, 3D).

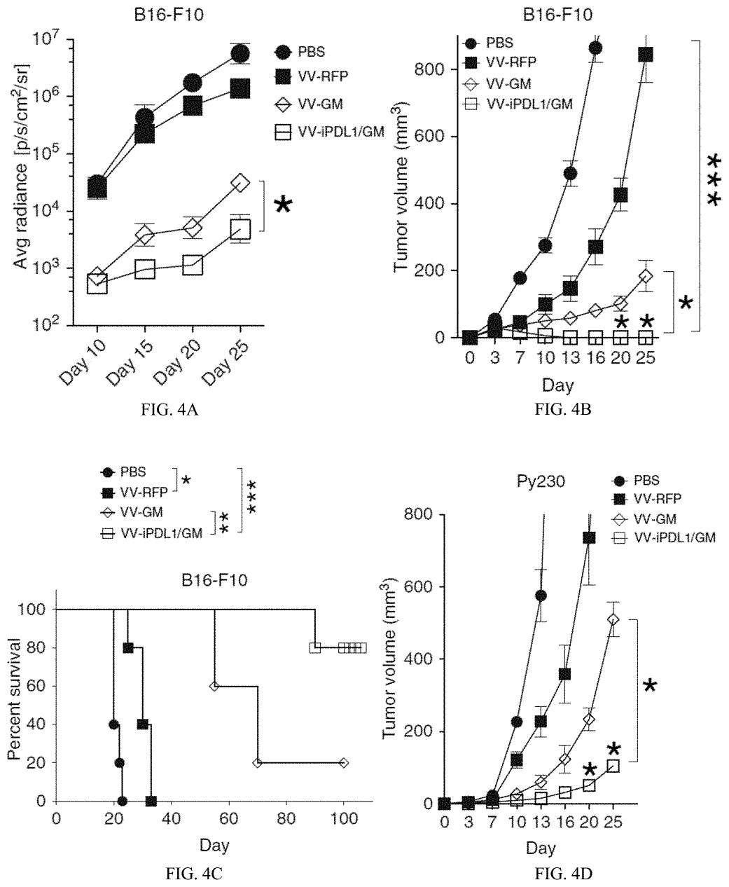

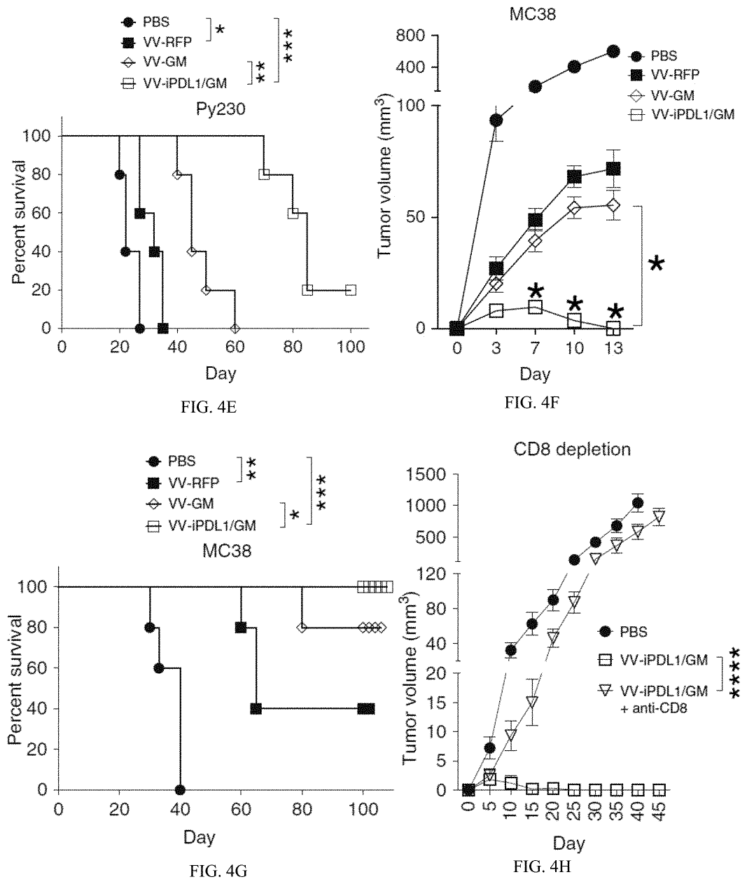

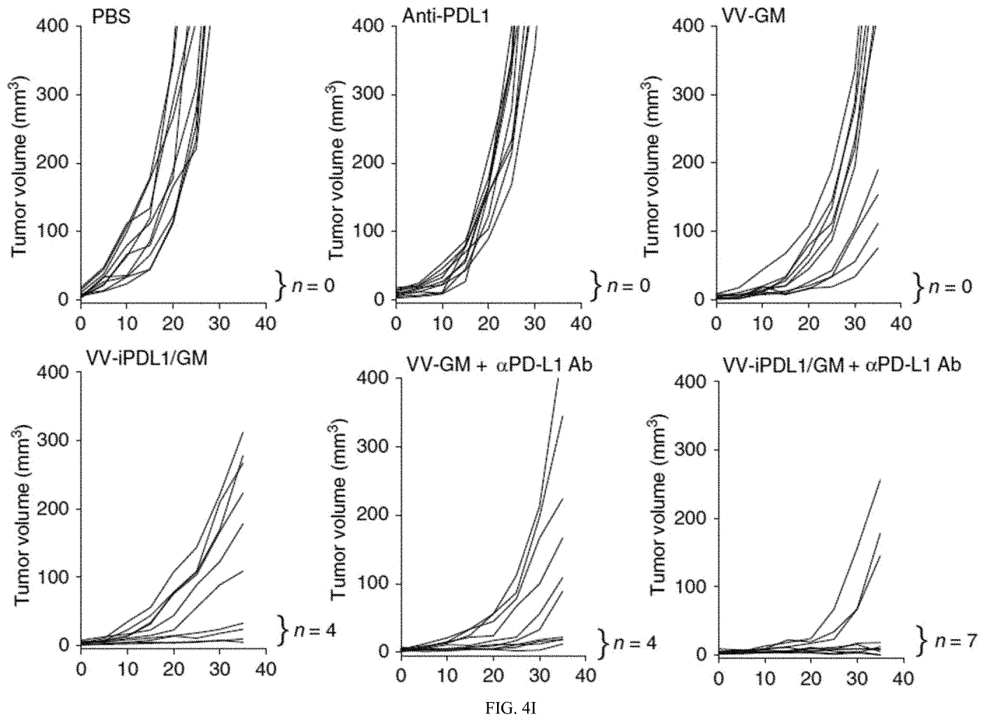

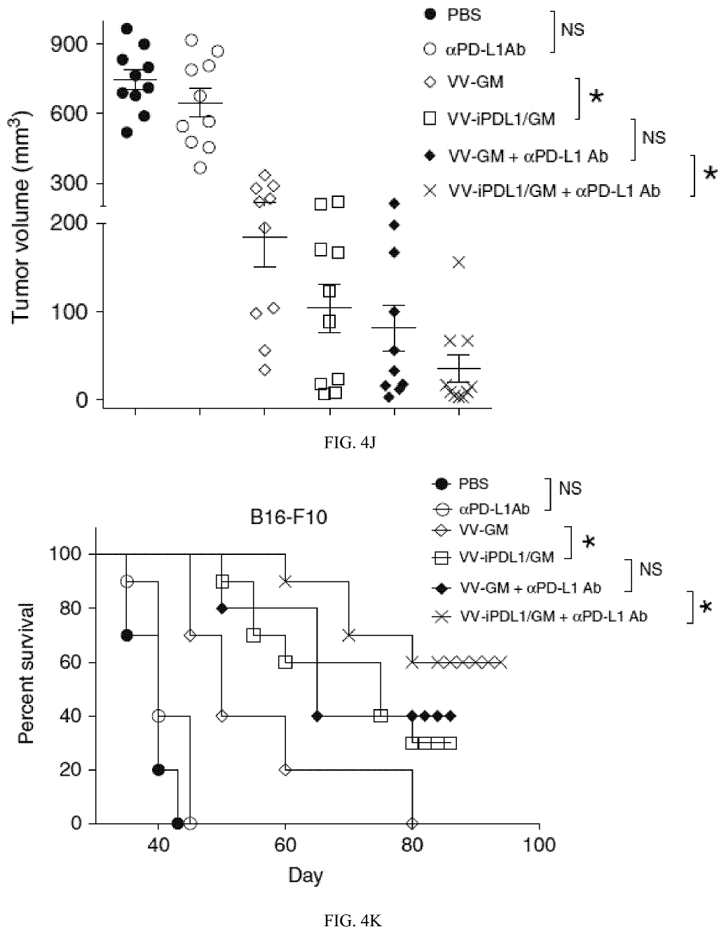

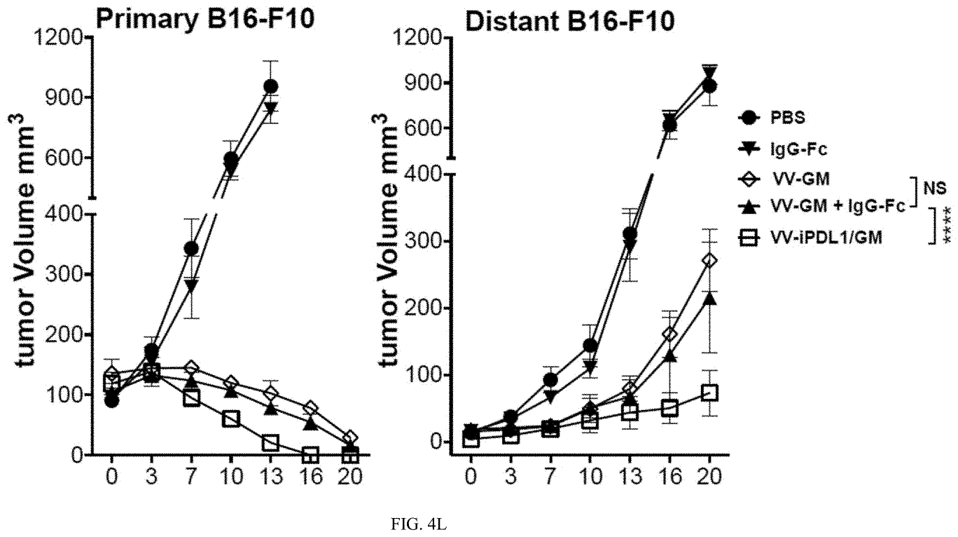

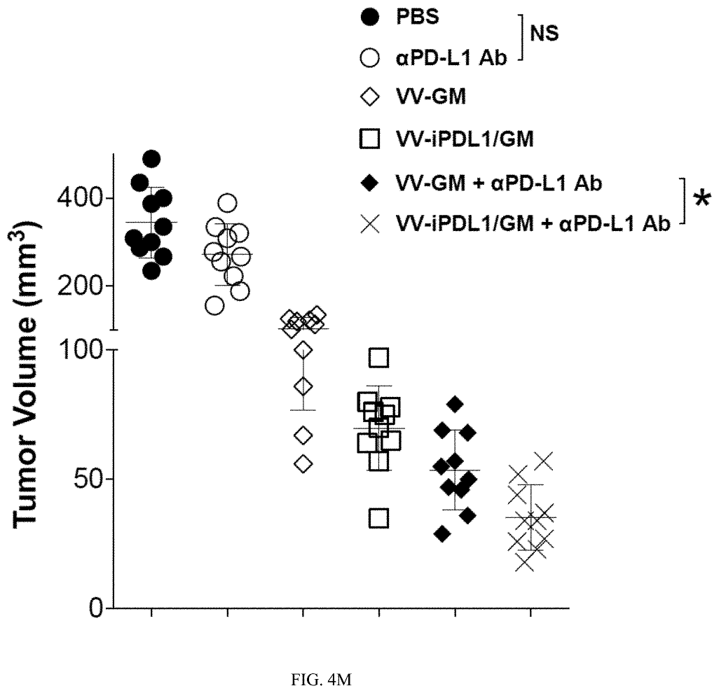

We then tested if an intratumoral injection with VV-iPDL1/GM is able to provoke a systemic antitumor response. Groups of C57BL/6 mice bearing B16-F10 tumors were treated with various VVs, and then inoculated with luciferase+ B16-F10 tumors on the contralateral flank. Bioluminescence imaging (FIG. 4A), caliper measurement (FIG. 4B), and survival curve (FIG. 4C) showed that VV-iPDL1/GM was more potent in inhibiting the growth of rechallenged homologous B16-F10 tumors, compared to VV-GM and VV-RFP. Furthermore, intratumoral injections with VV-iPDL1/GM were also more potent in inhibiting the growth of rechallenged homologous Py230 tumors and MC38 tumors, compared to the intratumoral injections with VV-GM or VV-RFP (FIG. 4D-4G). In vivo CD8 T cell depletion significantly abolished the systemic antitumor activity in VV-iPDL1/GM-treated tumor-bearing mice (FIG. 4H).

We further tested if an intratumoral injection with VV-iPDL1/GM is able to provoke a systemic antitumor response against established tumor growth. B16-F10 melanoma cells were implanted to the left and right flanks of C57B/6 mice. When tumor volumes reached ˜100 mm³, the tumors on the left flank were injected with VV-GM, VV-iPDL1/GM, or PBS without or with i.v. injections of a neutralizing anti-PD-L1 antibody (left treated tumor volumes determined on day 10 for individual tumors are shown in FIG. 2J). Intratumor injections of VV-iPDL1/GM more efficiently inhibited the growth of the untreated, distant B16-F10 tumors than intratumor injections of VV-GM did (FIGS. 4I, 4J). Co-injections of IgG Fc and VV-GM did not substantially enhance antitumor activities of VV-GM against treated and untreated, distant tumors (FIG. 4L). The data indicated that IgG Fc domain alone unlikely contributed to the enhanced antitumor activity of VV-iPDL1/GM. We observed that systemic injections of the neutralizing anti-PD-L1 antibody alone were unable to control the growth of the weakly immunogenic B16-F10 melanoma (FIGS. 4I, 4J). Interestingly, coadministrations with PD-L1 antibody enhanced the systemic antitumor activity of both VV-GM and VV-iPDL1/GM. However, coadministrations of PD-L1 antibody and VV-iPDL1/GM had more potent systemic antitumor activities than coadministrations of PD-L1 antibody and VV-GM (FIGS. 4I-4K, 4M). Collectively, these in vivo data demonstrate that intratumoral injections with the double-armed VV-iPDL1/GM alone or in combination with an anti-PD-L1 antibody are able to provoke potent, systemic antitumor responses.

### 1.4. Enhanced Tumor Infiltration and Activation of Immune Cells.

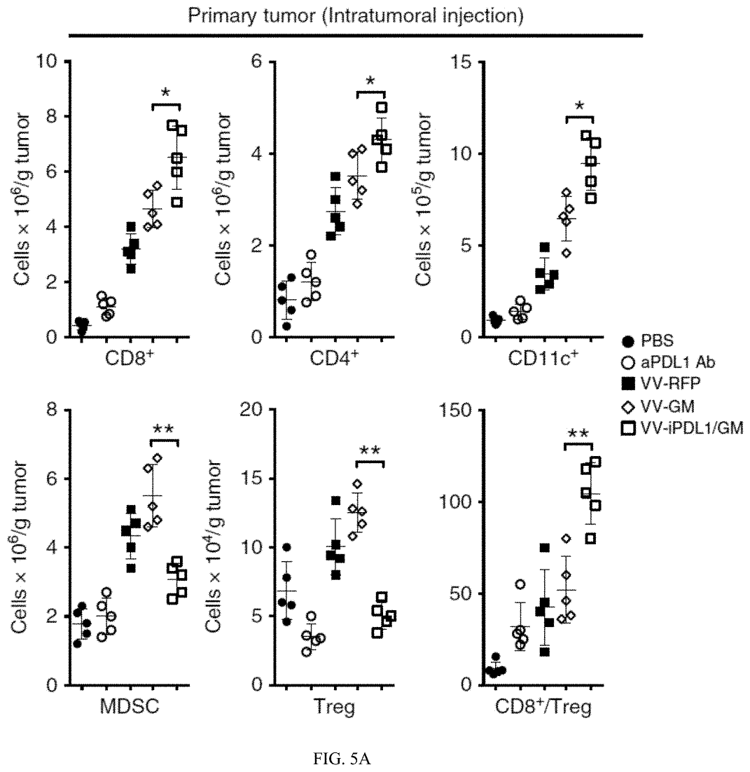

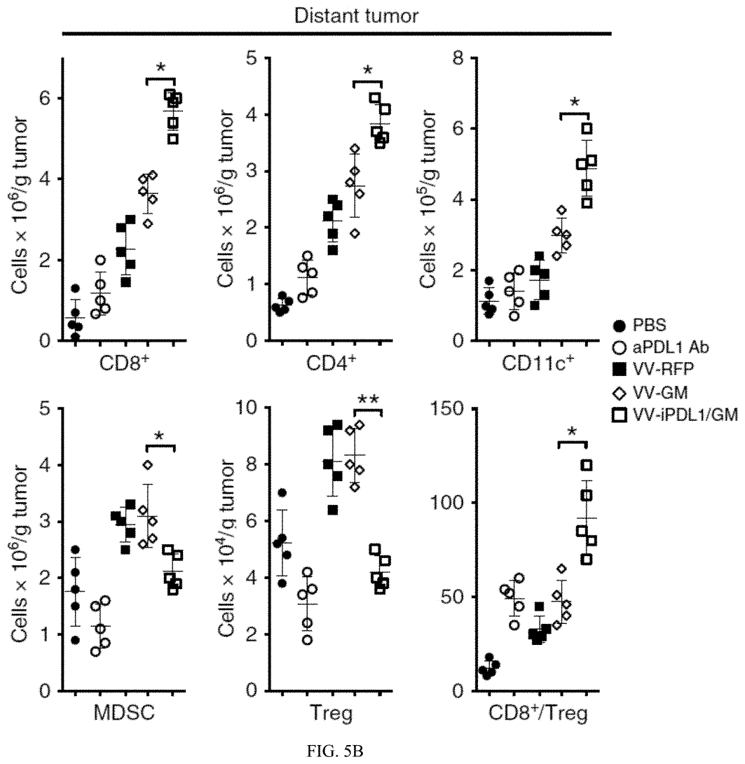

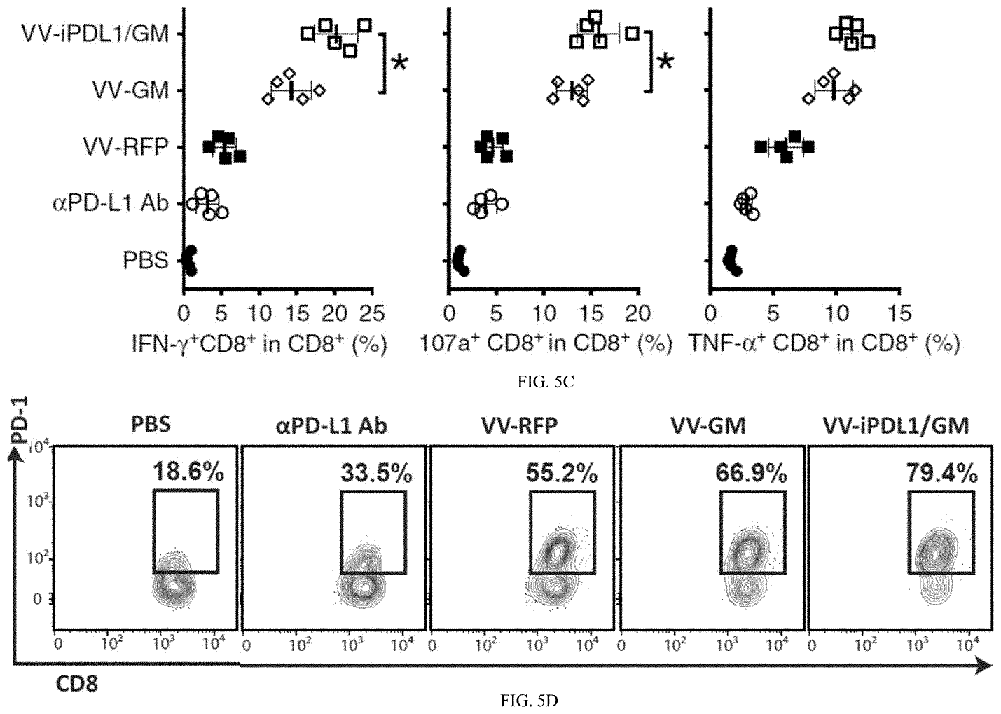

We analyzed the tumor infiltration of immune cells after intra-tumoral injections of VV-iPDL1/GM. Groups of MC38 tumor-bearing mice were treated with various VVs via intratumoral injections. One group of MC38-bearing mice was i.p. injected with anti-PD-L1 Ab (clone 10F.9G2) for comparison. VV-intratumoral injections significantly enhanced the tumor infiltration of CD45+ hematopoietic cells, especially the injections of VV expressing GM-CSF. VV-GM injection enhanced composition of MDSC-containing cells (CD11b+Gr-1+, 46%) in the CD11b+ population. In contrast, VV-iPDL1/GM injection greatly reduced MDSCs to 23% of the CD11b+ population, which was consistent with the reduced absolute MDSC numbers of VV-iPDL1/GM-treated or distant tumors (FIGS. 5A, 5B), indicating the ability of VV-iPDL1/GM to block the PD-1/PD-L1 interaction and decrease tumor-associated immune suppressive cells. Moreover, VV-iPDL1/GM significantly enhanced dendritic cell (DC; CD11c+) content in the infiltrates compared with control VV-RFP (FIGS. 5A, 5B).

We subsequently analyzed infiltrating lymphocytes in VV-treated tumors. VV injections enhanced the overall lymphocyte infiltration into tumor tissues. However, the double-armed VV-iPDL1/GM enhanced the percentages of CD8+ T cells, and CD4+ T cells, and PD-1+CD8+ T cells in the CD45+ infiltrates more significantly in comparison to control VV or single-armed VV-GM (FIG. 5D). The injection of VV-GM alone did not significantly affect Treg cells (CD4+FoxP3+) in tumor infiltrates, but the injection of VV-iPDL1/GM reduced Treg cells to a level lower than that in PBS-treated tumors, resulting in a robustly enhanced CD8+ T cells/Treg ratio (FIG. 5A). We further analyzed infiltrating lymphocytes in distant, untreated tumors. FIG. 5B also shows that the intratumoral injection with VV-iPDL1/GM enhanced the tumor infiltration and activation of lymphocytes and other immune cells in distant, untreated tumors. Moreover, tumor-infiltrating CD8+ effector T cells were more efficiently activated by VV-iPDL1/GM injections, as manifested by an enhanced expression of IFN-γ, TNF-α, and CD107a in response to the stimulation with tumor lysate-pulsed DCs (FIG. 5C). Altogether, these findings demonstrate that the double-armed VV-iPDL1/GM has the ability to alter the tumor microenvironment by enriching the tumor infiltration of immune cells, reducing immune suppressive cells in the tumors, and activating tumor-infiltrating effector T cells.

### 1.5. Enhanced T Cell Responses Against Dominant and Subdominant Neoantigen Epitopes.

We tested whether an intratumoral injection of VV-iPDL1/GM is able to generate neoantigen-specific T cell responses. Recently, Yadav et al. identified MHC-I-restricted neoepitopes in MC38 tumor cells using whole-exome and transcriptome sequencing analysis combined with mass spectrometry. MC38 tumor-bearing mice were intratumorally treated with various VVs. Ten days after the last viral injection, splenocytes were harvested and analyzed for the neoepitopes-specific immune responses. Eleven mutant neoantigen epitopes were synthesized and used for this study (Table 2).

TABLE 2
Neoantigenic epitope peptides used in this study.

| | | | | IC₅₀ |
|---|---|---|---|---|
| | | MHC | IC₅₀ | (Wild |
| Name | Peptide | allele | (mutant) | Type) |
| Pepl | AALLNSA(G/V)L | H-2d^{b} | 3 nM | 52 nM |
| | (SEQ ID NO: 3)/(SEQ ID NO: 4) | | | |
| Pep2 | AQL(P/A)NDVVL | H-2D^{b} | 9 nM | 100 nM |
| | (SEQ ID NO: 5)/(SEQ ID NO: 6) | | | |
| Pep3 | MAPIDHT(A/T)M | H-2D^{b} | 30 nM | 102 nM |
| | (SEQ ID NO: 7)/(SEQ ID NO: 8) | | | |
| Pep4 | ASMTN(R/M)ELM | H-2D^{b} | 2 nM | 3 nM |
| | (SEQ ID NO: 9)/(SEQ ID NO: 10) | | | |
| Pep5 | SIIVFNL(V/L) | H-2K^{b} | 8 nM | 34 nM |
| | (SEQ ID NO: 11)/(SEQ ID NO: 12) | | | |
| Pep6 | SSP(D/Y)SLHYL | H-2D^{b} | 211 nM | 685 nM |
| | (SEQ ID NO: 13)/(SEQ ID NO: 14) | | | |
| Pep7 | (S/I)MTQHLEPI | H-2D^{b} | 78 nM | 29 nM |
| | (SEQ ID NO: 15)/(SEQ ID NO: 16) | | | |
| Pep8 | SAIRSYQ(D/Y)V | H-2D^{b} | 35 nM | 755 nM |
| | (SEQ ID NO: 17)/(SEQ ID NO: 18) | | | |
| Pep9 | VSPVND(V/L)DV | H-2d^{b} | 44 nM | 18 nM |
| | (SEQ ID NO: 19)/(SEQ ID NO: 20) | | | |
| Pep10 | MG(G/V)MNRRPI | H-2D^{b} | 77 nM | 841 nM |
| | (SEQ ID NO: 21)/(SEQ ID NO: 22) | | | |
| Pep11 | FM(A/S)CNLLLV | H-2D^{b} | 79 nM | 24 nM |
| | (SEQ ID NO: 23)/(SEQ ID NO: 24) | | | |

| | | | |
|---|---|---|---|
| HPV E7 | YMLDLQPETT (SEQ ID NO: 25) | H-2D^{b} | Irrelevant peptide |
| OVA₍₂₅₇₋₂₆₄₎ | SIINFEKL (SEQ ID NO: 26) | H-2K^{b} | Irrelevant peptide |

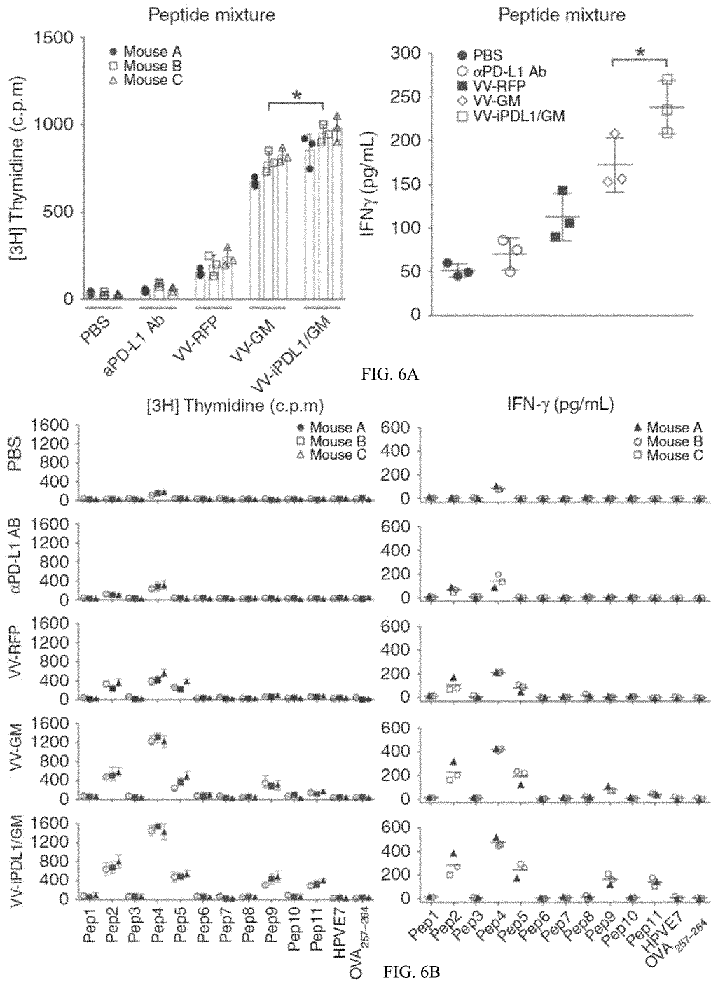

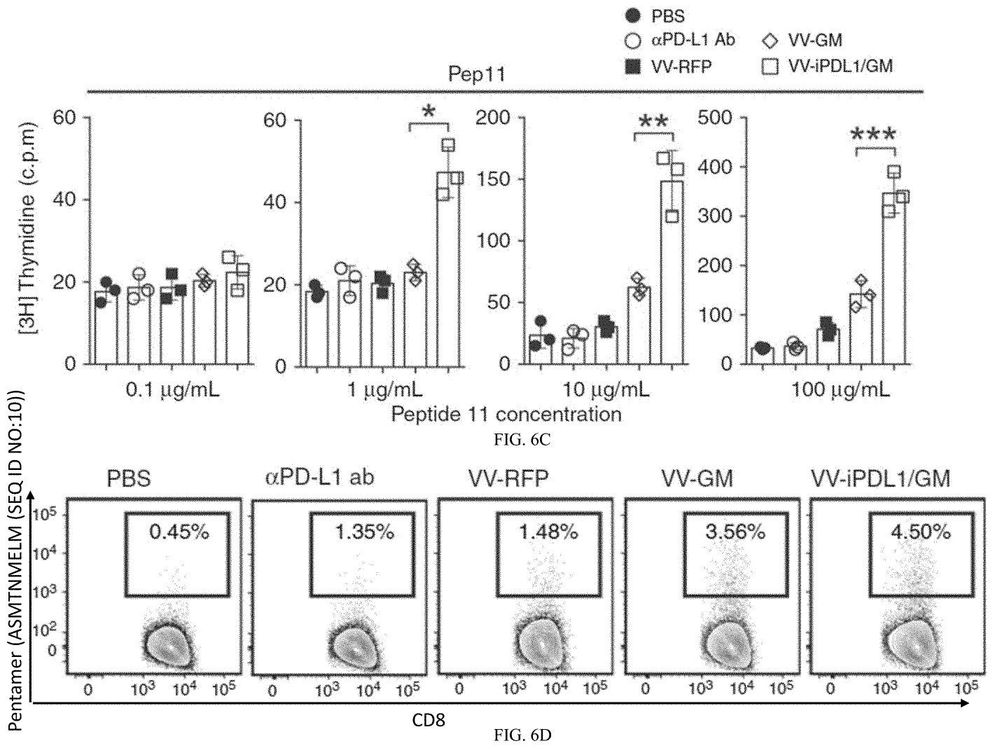

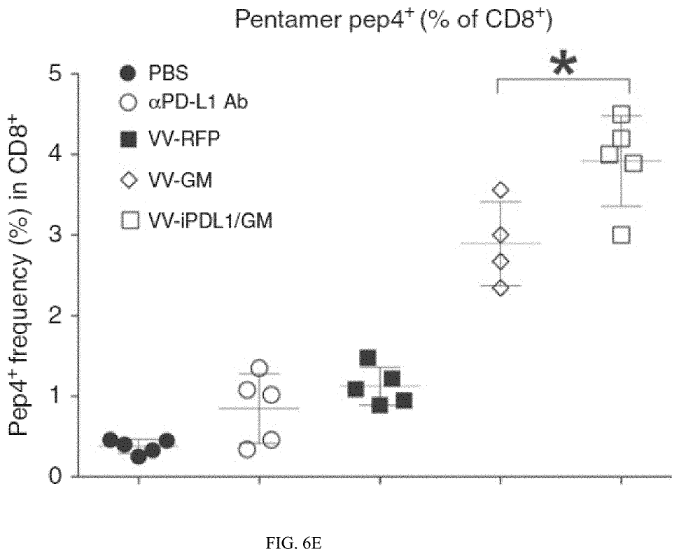

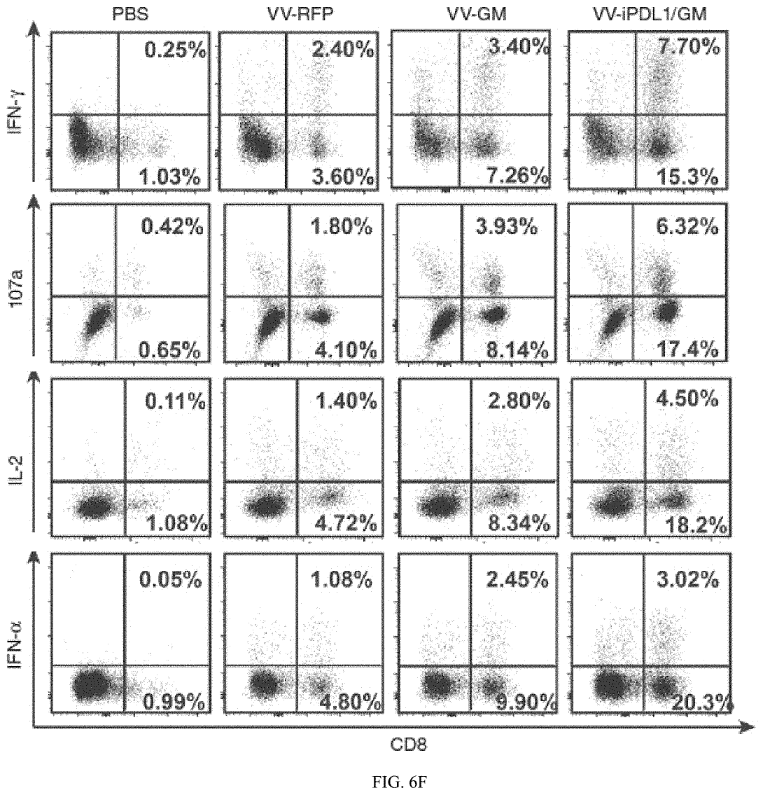

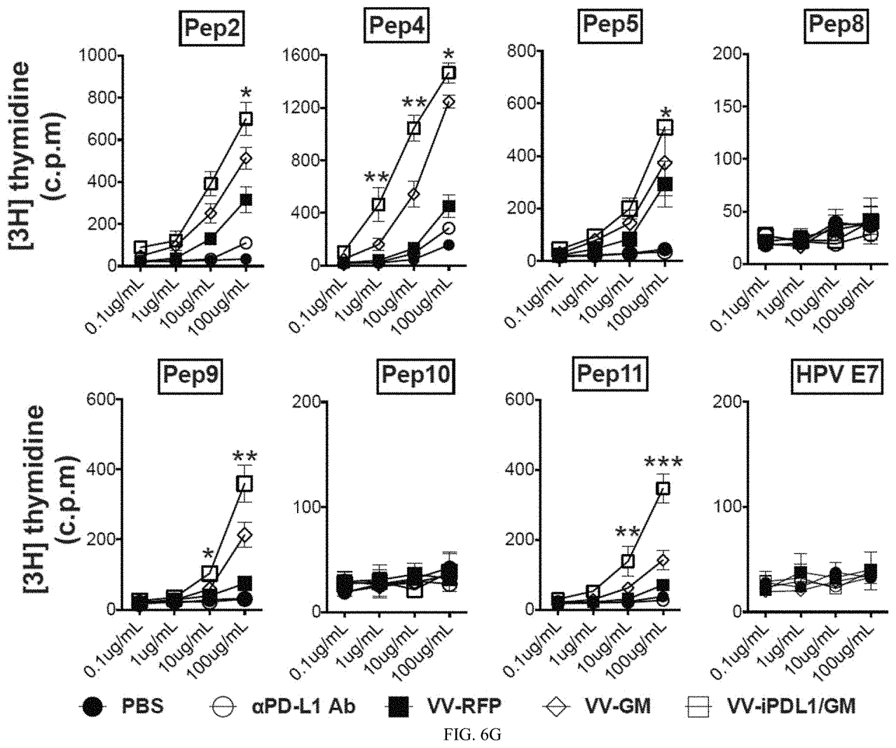

Neopeptides 1-6 were detected on the cell surface by the membrane protein purification and mass spectrometry method, while neopeptides 7-11 were not detected on the cell surface, probably due to the sensitivity of the detection method, or poor peptide processing and presentation. After intratumoral injections with VVs, the tumor-bearing mice exhibited an enhanced proliferation and cytokine (IFN-γ) secretion of splenic T cells compared with that in PBS-treated mice in response to stimulation with the 11 neopeptides mixture. However, the most potent splenic T cell responses against the neopeptides mixture were detected in the VV-iPDL1/GM-treated mice (FIG. 6A). Importantly, systematical (i.p.) administration of anti-PD-L1 antibody (200 μg) did not significantly induce neopeptide-specific T cell responses in the tumor-bearing mice. These data indicate the superior potency of VV-iPDL1/GM to activate neoantigen-specific T cell responses.

We then analyzed T cell responses in VV-treated mice against individual neoepitopes. VV-RFP enhanced the proliferation and cytokine production of splenic T cells of treated mice in response to neoepitopes 2, 4, and 5, compared to only neoepitope 2 or 4 in the PBS or anti-PD-L1 antibody-treated mice (FIGS. 6B, 6G). VV-GM significantly enhanced the T cell responses to neoepitopes 2, 4, and 5, and also additionally triggered T cell responding to neoepitope 9 and slightly to neoepitope 11. Compared with VV-GM, VV-iPDL1/GM further strengthened T cell responses against neoepitopes 2, 4, and 5, as well as the subdominant neoepitopes 9 and 11 (FIGS. 6B, 6G). Furthermore, splenic T cells from the VV-iPDL1/GM-treated mice showed responses against dominant neoepitope 2, 4, or 5 even at a very low peptide concentration (0.1 μg/mL or 1 μg/mL), and also showed responses against subdominant neoepitope 9 or 11 at a low concentration (10 μg/mL), in which splenic T cells from other VV-treated mice didn't show detectable responses (FIGS. 6C, 6G). Given the prominent neoepitope 4-specific T cell response detected in various VV-treated mice, neoepitope 4 peptide-MHC H-2Db-labeled pentamers were synthesized and used to analyze tumor-infiltrating neoantigen-specific T cells. Among the groups, the VV-iPDL1/GM-treated mice had maximal CD45+CD8+pentamer+ T cells in tumor infiltrates (FIGS. 6D, 6E), indicative of VV-iPDL1/GM injections being most efficacious in activating neoepitope 4-specific T cells in the tumor-bearing mice.

Even 40 days after the last VV injection when all tumors were gone, splenocytes of the VV-iPDL1/GM-treated mice showed the strongest response to neoepitope 4-loaded DC restimulation (FIG. 6F). These results demonstrate the ability of VV-iPDL1/GM to activate T cell responses against dominant and subdominant neoantigen epitopes.

### 1.6. Enhanced Tumor-Infiltrating DC Maturation and Neoantigen Presentation.

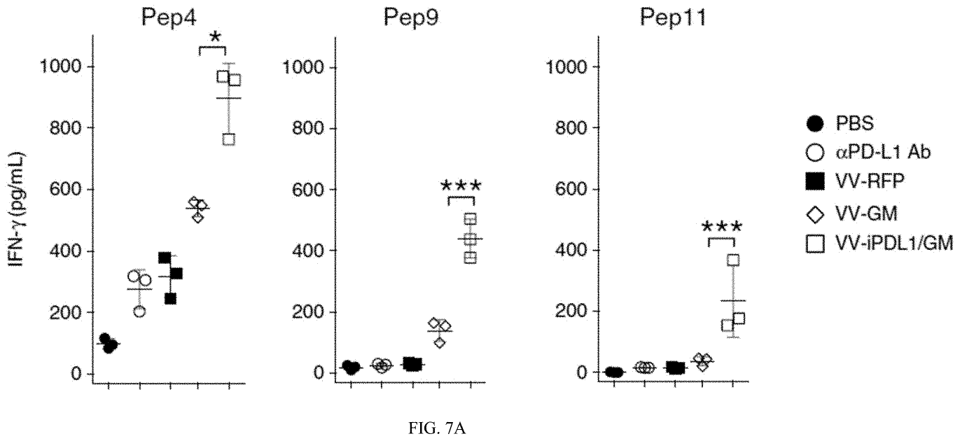

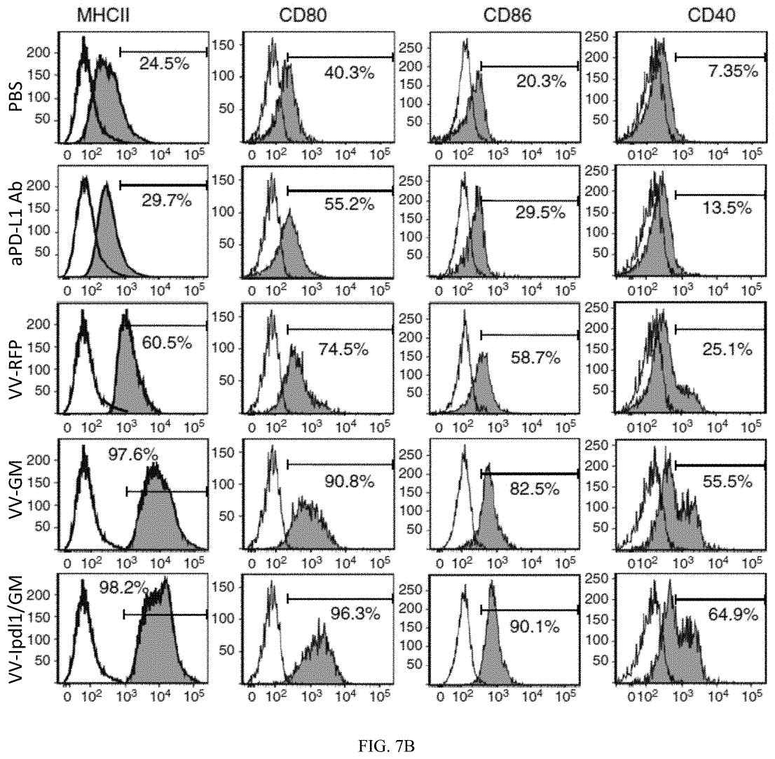

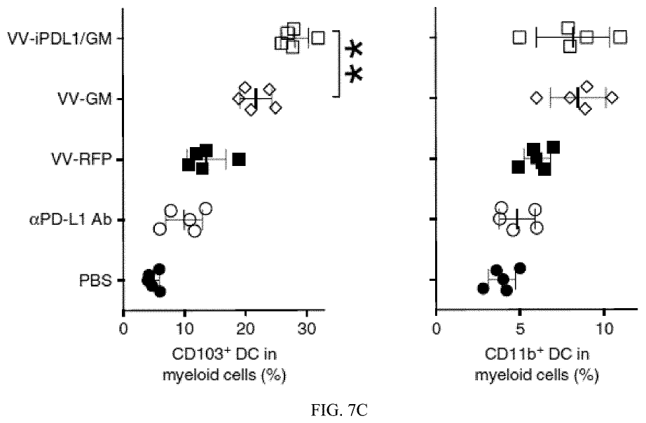

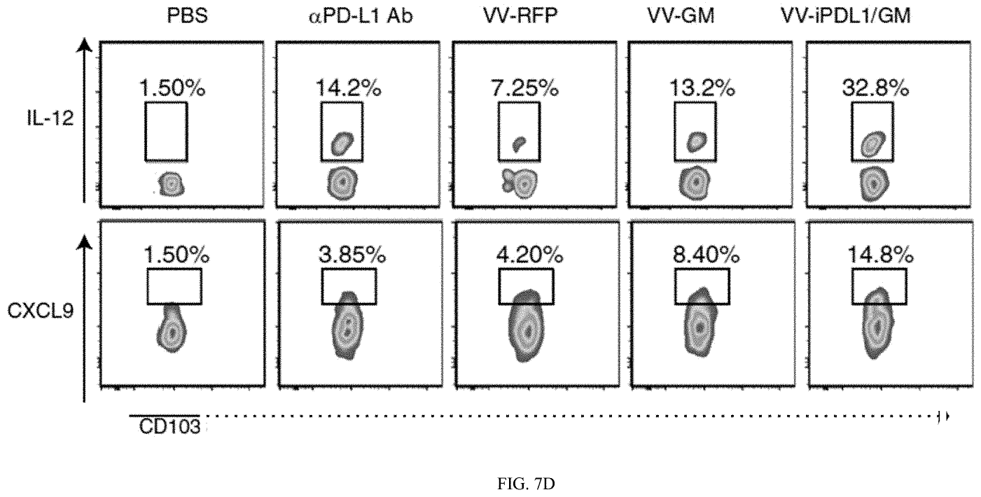

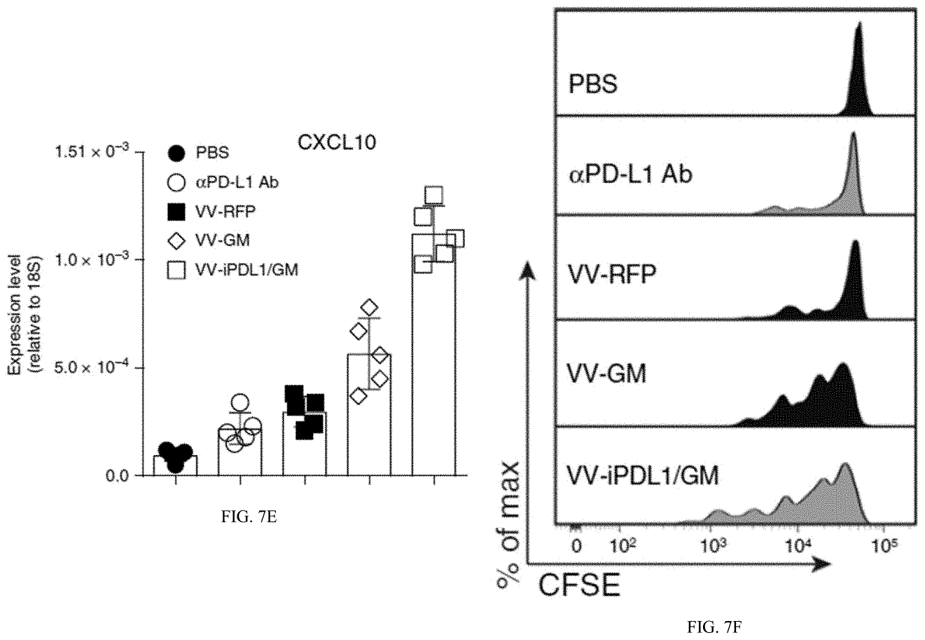

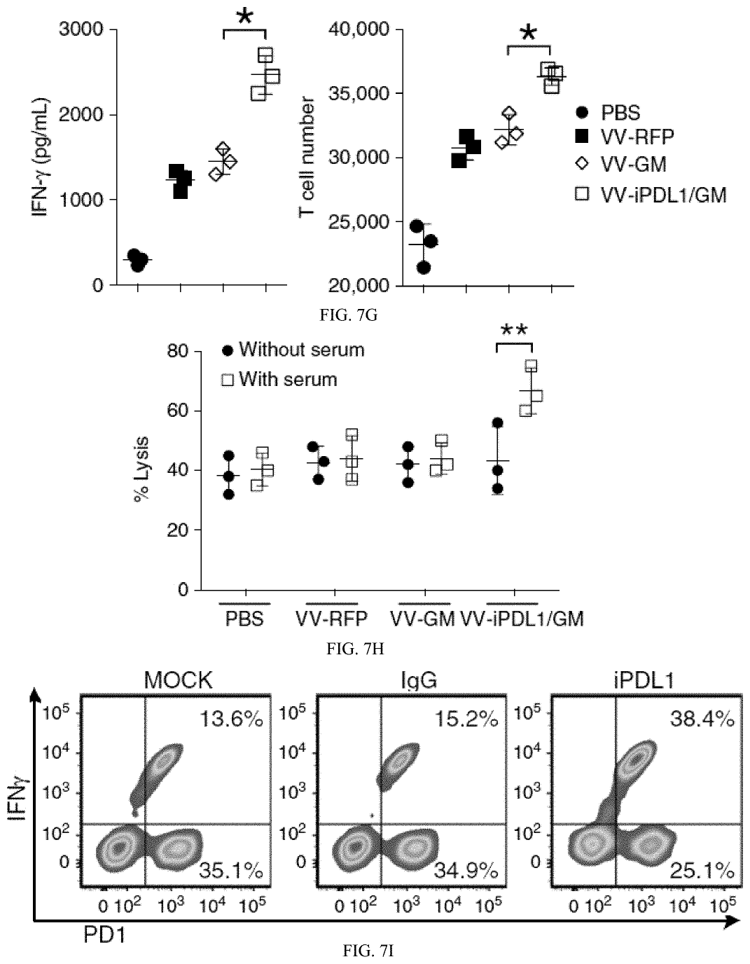

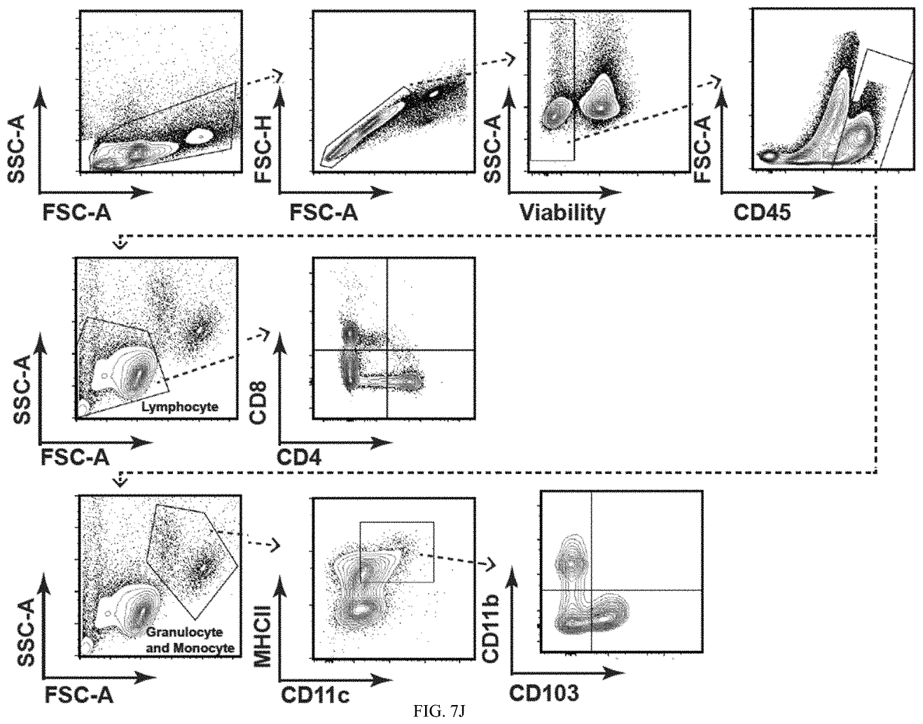

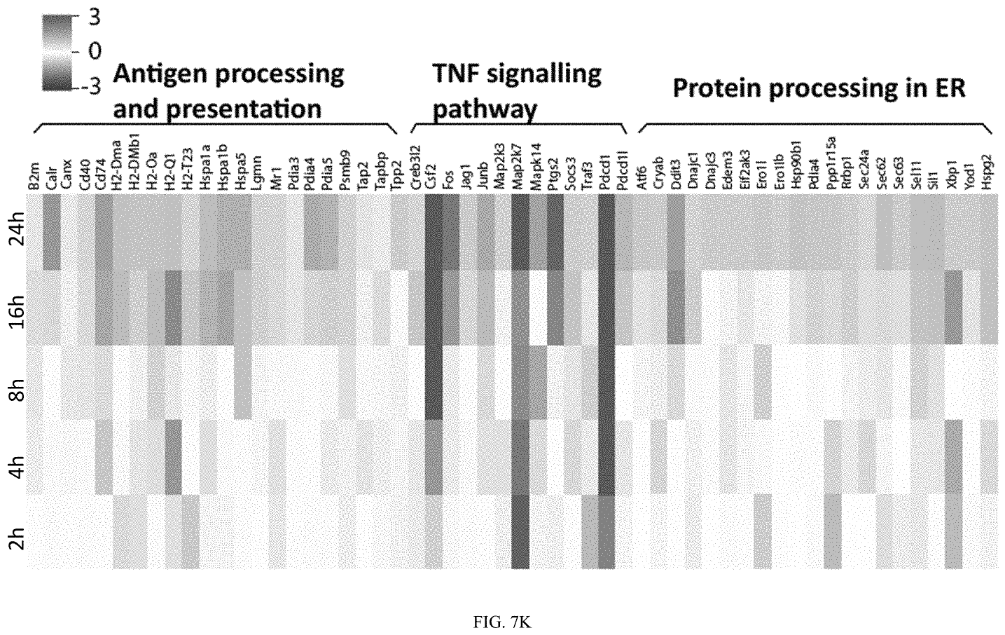

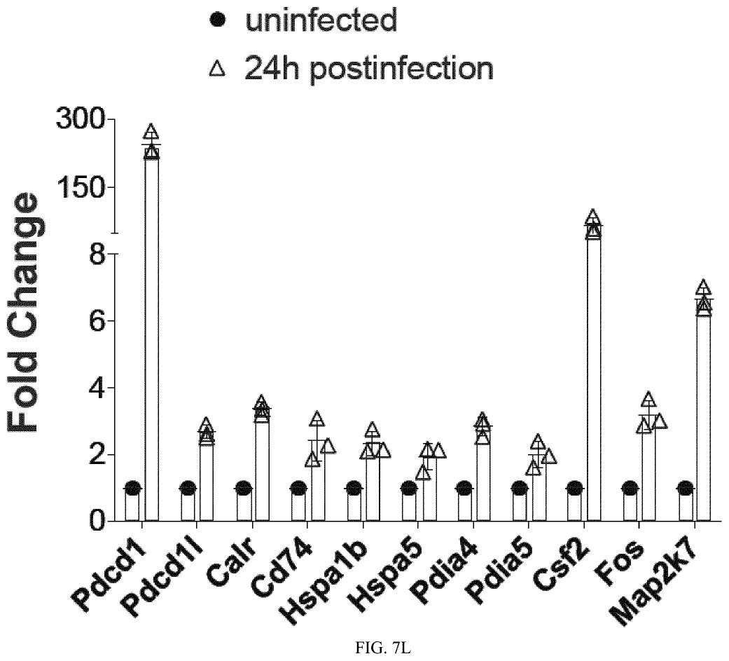

We further explored the mechanisms of the double-armed VV-iPDL1/GM to activate neoantigen-specific T cell responses. DCs are professional antigen-presenting cells with the ability to prime antigen-specific T cell responses. Thus, we compared the immunostimulatory potency of tumor-infiltrating DCs from various VV-treated mice. Tumor-infiltrating CD11c+ DCs isolated from VV-treated MC38 tumors were pulsed with neopeptides 4 (dominant), 9, and 11 (subdominant), and then cocultured with neoantigens-primed T cells isolated from mice immunized with the 11 neoepitope peptides mixture formulated with adjuvants. Tumor-infiltrating DCs from VV-iPDL1/GM-treated MC38 tumors had the enhanced potency to stimulate neoantigens-primed T cells (FIG. 7A). In comparison, tumor-infiltrating DCs from MC38 tumor-bearing mice receiving anti-PD-L1 antibody (i.v.) alone only had a much weaker stimulatory potency. We also observed that VV-iPDL1/GM significantly promoted tumor-infiltrating DC maturation, as evidenced by an increased expression of MHCII, CD80, CD86, and CD40 (FIG. 7B). A recent study revealed that CD103+ DCs are the main intratumoral myeloid cell population that transports antigens to the tumor-draining lymph nodes for activating T cells. The analysis of surface markers on the DC population showed that VV-iPDL1/GM injection significantly increased tumor-infiltrating CD103+ DCs, compared to VV-GM or VV-RFP (FIGS. 7C, 7J). IL-12 is an important cytokine in cross talk between DCs and T cells. Chemokines CXCL9 and CXCL10 direct effector T cell trafficking and tumor infiltration. The expression of IL-12, CXCL9, and CXCL10 in CD103+ DCs from VV-iPDL1/GM-treated tumors was elevated (FIGS. 7D, 7E). These data demonstrate that VV-iPDL1/GM injections likely enhanced tumor-infiltrating DC maturation and neoantigen presentation.

### 1.7. Enhanced Neoantigen Presentation on Tumor Cells, and CTL Effector Function.

During the effector phase of the antitumor response, activated T cells need to recognize neoantigen-presented tumor cells for their effector function. A poor neoantigen presentation and the expression of PD-L1 can render tumor cells resistant to CTL-mediated cytolysis. We performed an in vivo T cell proliferation assay, in which neoepitopes-primed T cells were adoptively transferred into the various VV-treated MC38-bearing mice. A higher efficiency in neoepitopes-primed T cell proliferation in vivo was observed in VV-iPDL1/GM-treated MC38-bearing mice (FIG. 7F). We tested the ability of neoantigen-specific T cells to recognize MC38 tumor cells infected with various VVs in vitro. MC38 tumor cells were infected with VV-iPDL1/GM or control VVs, and after washing, then cocultured with neoantigens-primed T cells isolated from mice immunized with the 11 neoepitope peptides mixture formulated with adjuvants. VV-iPDL1/GM-infected MC38 tumor cells were more potent in stimulating the proliferation and cytokine production of the neoepitopes-primed T cells (FIG. 7G), indicating that the neoepitopes-primed T cells more efficiently recognize and interact with the neoepitopes-presented, VV-iPDL1/GM-infected tumor cells. We further tested the role of the secreted iPDL1 in enhancing tumor cell immunogenicity. MC38 tumor cells without VV infection were cocultured with the neoepitopes-primed T cells isolated from mice immunized with the 11 neoepitope peptides mixture in the presence of sera from tumor-bearing mice treated with various VVs. FIG. 7H shows that only sera from VV-iPDL1/GM-treated mice were able to enhance the cytolytic activity of neoantigens-primed T cells against various VV-infected MC38 tumor cells. Moreover, it was observed that higher IFN-γ+ frequency of PD-1+ CD8+ T cells isolated from VV-treated MC38 tumor cell suspensions in the in vitro coculture with MC38 tumor cells in the presence of purified iPDL1 in comparison to the presence of control IgG (FIG. 7I), indicating the role of secreted iPDL1 in overcoming the immunosuppression of PD-L1+ tumor cells. In addition, our preliminary data showed the upregulation of the expression of TNF signaling genes and protein processing genes in VV-iPDL1/GM-infected tumor cells by RNA-Seq and qRT-PCR (FIGS. 7K and 7L, Table 3).

TABLE 3
List of primer sequence.

| | | |
|---|---|---|
| 18S | 5′-cggctaccacatccaaggaa-3′ | 3′-gctggaattaccgcggct-5′ |
| | (SEQ ID NO: 27) | (SEQ ID NO: 28) |
| Cxcl10 | 5′-gctgccgtcattttctgc-3′ | 3′-tctcactggcccgtcatc-5′ |
| | (SEQ ID NO: 29) | (SEQ ID NO: 30) |
| Calr | 5′-aaaggaccctgatgctgccaag-3′ | 3′-tgttcggtctcgtgtagggact-5′ |
| | (SEQ ID NO: 31) | (SEQ ID NO: 32) |
| Cd74 | 5′-gctggatgaagcagtggctctt-3′ | 3′-ggtccttcttcagtcggtgtag-5′ |
| | (SEQ ID NO: 33) | (SEQ ID NO: 34) |
| Hspa1b | 5′-acaagtcggagaacgtgcagga-3′ | 3′-gaagtggtggatgagcctgttg-5′ |
| | (SEQ ID NO: 35) | (SEQ ID NO: 36) |
| Hspa5 | 5′-tgtcttctcagcatcaagcaagg-3′ | 3′-ttcggacaggtccttcacaacc-5′ |
| | (SEQ ID NO: 37) | (SEQ ID NO: 38) |
| Pdia4 | 5′-gaccagtttgtgaaggagcactc-3′ | 3′-acttcaggaggtgcctctacga-5′ |
| | (SEQ ID NO: 39) | (SEQ ID NO: 40) |
| Csf2 | 5′-aacctcctggatgacatgcctg-3′ | 3′-tcgtcccagatgccccgttaaa-5′ |
| | (SEQ ID NO: 41) | (SEQ ID NO: 42) |
| Fos | 5′-gggaatggtgaagaccgtgtca-3′ | 3′-cccttgccttattctaccgacg-5′ |
| | (SEQ ID NO: 43) | (SEQ ID NO: 44) |
| Map2k7 | 5′-tcaggtgtggaagatgcggttc-3′ | 3′-gagttctcggtactgacgggaa-5′ |
| | (SEQ ID NO: 45) | (SEQ ID NO: 46) |
| Pdcd1 | 5′-cggtttcaaggcatggtcattgg-3′ | 3′-ccttcgttcctgctgtgagact-5′ |
| | (SEQ ID NO: 47) | (SEQ ID NO: 48) |
| Pdcd1l | 5′-tgcggactacaagcgaatcacg-3′ | 3′-gctcccaataggtcttcgactc-5′ |
| | (SEQ ID NO: 49) | (SEQ ID NO: 50) |

A possible role of neoantigen presentation enhanced by VV-iPDL1/GM infection cannot be ruled out. These data demonstrate the enhanced neoantigen presentation on tumor cells by VV-iPDL1/GM infection for enabling neoantigen-specific CTL effector functions.

Immunosuppressive tumor microenvironments, due to the lack of the “danger signals” of pathogen-associated molecular pattern (PAMP) molecules, and the expression of immune checkpoints, such as PD-L1, on tumor cells, T cells, and DCs, inhibit the priming or activation of T cell responses against tumor neoantigens. The engineered oncolytic virus generated in this study is able to produce the PD-L1 inhibitor, and bind to PD-L1+ tumor cells and immune cells. It is tempting to postulate that the secretory iPDL1 in combination with the viral oncolysis-mediated, immunogenic cell death and the release of viral PAMP molecules from infected cells may lead to the enhanced DC maturation and neoantigen presentation in the tumor microenvironment, and the systemic activation of tumor neoantigen-specific T cell responses. Thus, this study demonstrates that secretory PD-L1 inhibitors, GM-CSF, and viral oncolysis work together to promote neoantigen presentation and activate tumor neoantigen-specific T cell response, representing a potent, individual tumor-specific oncolytic immunotherapy.

An interesting finding of this study is the ability of the armed oncolytic virus to activate T cell responses against subdominant neoantigen epitopes. T cell responses are primed or activated by DCs, which present a repertoire of MHC-associated peptides. The tumor neoantigen repertoire derived from mutated gene products are presented to T cells after DCs capture and process antigens, load processed peptides onto MHC-I molecules via cross-pre-sentation, and go through the maturation processing associated with the upregulation of costimulatory molecule and cytokine expression triggered by PAMP molecules. It is postulated that mutated proteins are processed and presented by the MHC molecule as neoantigens to T cells at different levels of efficacy such that certain mutated epitopes are efficiently processed and presented (dominant neoantigen epitopes), whereas others are poorly processed and presented at subthreshold levels, especially the microenvironment with PD-L1 expression on APCs (sub-dominant or cryptic neoantigen epitopes). The co-action of viral oncolysis, GM-CSF, and PD-L1 inhibition of DCs and T cells by this engineered oncolytic virus may enhance the ability of DCs to present the neoantigen repertoire to T cells, leading to the activation of T cell responses against both dominant and subdominant neoantigenic epitopes.

During the effector phase of antitumor T cell responses, the poor processing and presentation of neoantigenic epitopes and the expression of PD-L1 on tumor cells can inhibit CTL effector functions. The results of this study demonstrated that an intra-tumoral injection with this engineered oncolytic virus promoted the tumor infiltration and activation of neoantigen-specific T cells and immune cells, as well as neoantigen presentation on tumor cells via the inhibition of PD-L1 by the secreted PD-L1 inhibitors, leading to the systemic rejection of both the treated tumor and distant tumors.

Before the present disclosure, oncolytic virus therapies so far only showed limited efficacy in cancer patients. Previous studies did not investigate the ability and mechanisms of the armed oncolytic viruses to activate tumor neoantigen-specific T cell responses. Recent studies found that the reactive upregulation of PD-L1 expression in the tumor microenvironment after virus administration caused the tumor resistance to oncolytic immunotherapy. The production of PD-L1 inhibitors by this engineered oncolytic virus generated in this study is conceived to overcome this problem. Moreover, this oncolytic virus, which activates the neoantigen-specific T cell response by the co-action of PD-L1 inhibition, GM-CSF, and viral oncolysis in the tumor microenvironment may be advantageous to the therapies with PD-1/PD-L1 antibodies.

In summary, this engineered armed oncolytic virus with the ability to activate neoantigen-specific T cell responses by the co-action of viral immunogenic oncolysis, GM-CSF function, and PD-L1 inhibition on tumor cells and immune cells provides a potent, individual tumor-specific oncolytic immunotherapy, which could be therapeutically used alone or in combination with immune checkpoint inhibitors, targeted therapy, and chemotherapy for cancer patients, especially those resistant to PD-1/PD-L1 blockade therapy.

### 1.8. Procedures and Materials

Cell lines: Human embryonic kidney cell line 293T, osteosarcoma HUTK-143B, monkey kidney fibroblasts CV1, murine adenocarcinoma Py230, murine mela-noma B16-F10, and murine lymphoma EL4 were purchased from the American Type Culture Collection (ATCC). Murine colon adenocarcinoma cells MC38 was purchased from Kerafast. All the adherent cells were cultured in complete Dulbecco's modified Eagle's medium supplemented with 10% heat-inactivated fetal bovine serum (FBS) and 1% penicillin-streptomycin-glutamine 100× (Thermo, cat. no.: 10378016). T cells and splenocytes were grown in RPMI with 10% of heat-inactivated FBS, 10 mM HEPES, 1 mM sodium pyruvate, 0.05 mM β-mercap-toethanol, 1% penicillin-streptomycin-glutamine, and 1× minimal essential med-ium nonessential amino acids. Cells were maintained in an incubator at 37° C. and 5% CO₂.

Antibodies: The antibodies used in the study included: anti-CD16/32 (clone: 93, Biolegend, 1:100), anti-PD-L1 (APC or PE-cy7, clone: 10F.9G2, Biolegend; clone: MHI5, eBioscience, 1:100), anti-IgG2a-Fc (Polyclonal, Thermo, 1:500), anti-CD45 (BV421 or PE, clone: 30-F11, Biolegend, 1:500), anti-CD11c (PE or APC, clone: HL3, BD Biosciences, 1:100), anti-CD11b (eF450 or PE-cy5, clone: M1/70, BD Biosciences, 1:100), anti-CD103 (FITC, clone: 2E7, Biolegend, 1:100), viability dye (BV510 or UV450, Tonbo Biosciences, 1:1000), anti-CD3 (FITC or Pacific Blue, clone: 17A2, Biolegend, 1:1000), anti-CD4 (PE or PE-cy5, clone: RM4-5, BD Biosciences, 1:500), anti-CD8 (FITC, APC, or APC-cy7, clone: 53-6.7, Biolegend, 1:1000), anti-Gr-1 (PE or PE-cy7, clone: RB6-8C5, Biolegend, 1:100), MHCII (Pacific Blue, FITC, or PE, clone: M5/114.15.2, Biolegend, 1:100), anti-FoxP3 (APC or PE, clone: FJK-16s, Biolegend, 1:100), anti-IFN-γ (APC, clone: XMG1.2, Bio-legend, 1:100), anti-107a (FITC, clone: 1D4B, Biolegend, 1:500), anti-TNFα (PE, clone: MP6-XT22, BD Biosciences, 1:100), anti-IL-2 (PerCP−cy5.5, clone: JES6-5H4, Biolegend, 1:100), anti-IL-12 (PE-cy7, clone: C15.6, Biolegend, 1:100), anti-CXCL9 (AF647, clone:MIG-2F5.5, Biolegend, 1:100), anti-CD80 (PE-cy5, clone: 16-10A1, Biolegend, 1:100), anti-CD86 (PE-cy7, clone: GL-1, Biolegend, 1:100), and anti-CD40 (PE, clone 3/23, Biolegend, 1:100), anti-CD31 (FITC, clone: 390, Biolegend, 1:100), anti-Ter119 (APC, clone: TER-119, Biolegend, 1:100), IRDYE® 800CW Goat anti-Mouse IgG Secondary Antibody (Polyclonal, Li-cor), anti-CD40 (cat. no.: BP0016-2, Lot: 671717N1, BioXcell), and anti-PD-L 1 (clone: 10F.9G2, BioXcell) Pentamer H-2Db-ASMTNMELM-PE was provided by ProImmune Inc (1:100).

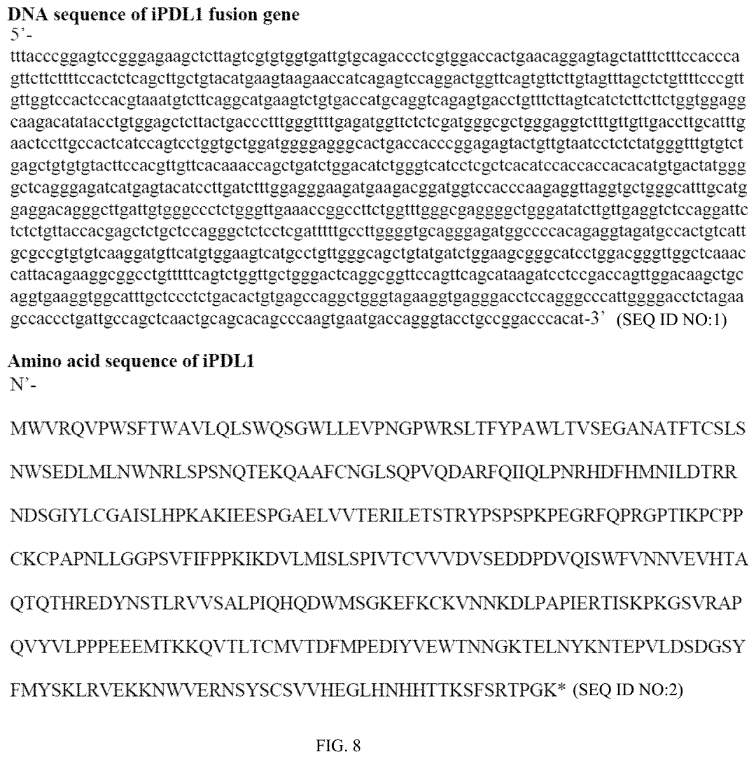

Recombinant VV generation and purification: VV shuttle vector pSel-DsRed2N1 pSel-DsRed2N1 was used to construct the recombinant shuttle vectors for coexpressing iPDL1 (FIG. 8) under the control of the VV Pse/1 promoter and GM-CSF under the control of the VV p7.5 later early promoter (FIG. 1A). To generate recombinant double-deleted (TK and Vaccinia growth factor) vaccinia viruses (ddVVs), a vgf gene-deleted WR strain VV, vSC20 ((VGF-virus with lacZ gene insertion), was used as a parental virus for homologous recombination. In brief, CV1 cells were infected with vSC20 at multiplicity of infection (MOI) of 0.1 for 2 h and then transfected with one of the recombinant shuttle plasmids. Cell extraction solution was used to infect HUTK-143B cells in the presence of 50 μg/mL bromodeoxyuridine (Sigma B5002). Three RFP-positive plaques were isolated, resuspended and further infect HUTK-143B cells for three more cycles of plaque selection until all plaques were RFP positive. The dislodged virus-infected cells were harvested with supernatants discarded by 5 min 1000×g centrifugation. The cells resuspended in 1-2 mL chilled 10 mM Tris buffer (pH=9.0) were sonicated for 1 min in water bath, and frozen/thawed for three times in dry ice/ethanol bath. The nucleus-free cell lysate was carefully layered in an ultracentrifuge tube appropriate for an ultra-centrifuge SW41 rotor prelayered with 2 mL of a 40% sucrose solution, and centrifuged at 20,000×g for 2 h at 4° C. without brakes. The white pellets at the bottom of the tube after ultracentrifugation resuspended in 200 μL to 1 mL 10 mM Tris buffer were stored at −80° C. and further used for animal study.

Titration of viruses: HuTK-143B cells (2×10⁵) were seeded into 12 well plates for 24 h. VVs with tenfold serial dilutions were added onto the cell monolayer. After 1 h incubation with rocking, the cells were gently added with 2 mL culture media and incubated for 24-48 h. The cells were washed and fixed in 0.1% crystal violet in 20% ethanol. The plaques were counted under microscope.

Western blot: A total of 5×10⁶ cells cultured in six-well plates were infected with indicated VVs at MOI=2. After incubation for 48 h, supernatants were harvested and clarified by centrifugation at 10,000×g for 2 min. Cells were lysed in 1× RIPA buffer (Sigma-Aldrich, St Louis, Mo.) and 1× mammalian protease inhibitor (Sigma-Aldrich, St Louis, Mo.) for 15 min on ice and clarified by centrifugation at 10,000×g for 2 min. Samples of both supernatants and cell lysates were mixed with 6× sodium dodecyl sulfate (SDS) sample buffer (Bioworld, Dublin, Ohio) and electrophoresed in a 4-20% gradient SDS-polyacrylamide gel (Thermo, Waltham, Mass.). The fractionated protein samples are transferred onto 0.2 μm nitrocellulose membrane (Thermo, Waltham, Mass.). The nitrocellulose membrane is blocked for 30-60 min at room temperature (RT) in TBS buffer (Bio-Rad, Irvine, Calif.) containing 5% nonfat milk. Immunodetection of iPDL1 is performed by incubation with RD800-conjugated goat anti-mouse IgG antibody (Licor, Lincoln, Nebr.) at RT for 1 h, or with rat anti-mouse PD-1 (Biolegend, San Diego, Calif.) at 1 μg/mL for overnight at 4° C. followed by with an RD800-conjugated anti-Rat IgG (Licor, Lincoln, Nebr.). The blots are detected with an Odyssey Imager (LI-CON, Lincoln, Nebr.).

Enzyme-linked immunosorbent assay: Tumor cells were infected with indicated viruses at MOI=2. After 24, 48, or 72 h, supernatants of the tumor cell cultures were collected. Mouse serum was collected at indicated times after intratumoral injection of indicated VVs. Serum iPDL1 or GM-CSF was determined using mouse PD-1 DuoSet ELISA kit (R&D, Minneapolis, Minn.) or mouse GM-CSF ELISA kit (Biolegend, San Diego, Calif.).

MTT assay: Tumor cells seeded in a 96-well plate were infected with indicated VVs at various MOIs in triplicate. The viability of tumor cells was determined using MTT assay (ATCC, Manassas, Va.) following the manufacturer's instruction. Optical density was read at 490 nm wavelength on a VersaMax microplate reader. The viability of the infected tumor cells was calculated as a percentage relative to the mock-infected cells58. Data=mean±SD.

BM-derived DC differentiation assay: Freshly isolated BM cells from mice were cultured in complete RPMI1640 media supplemented with 10% FBS, 20 ng/mL GM-CSF, and 40 ng/mL IL-4 for 72 h. Adherent or loosely adherent cells were collected, resuspended in culture media supplemented with 100 ng/mL IL-4 (Peprotech, London, UK), and aliquoted into 12-well tissue culture plate. A total of 100 μL of the supernatants of various VVs-infected cells (0.1 μm filtered) were added. A total of 50 ng/mL commercial GM-CSF (Peprotech, London, UK) was added as a positive control. All the cells were cultured for an additional 72 h and then analyzed by flow cytometric staining with APC-anti-CD11c.

iPDL1 protein purification: HUTK-143B cells were infected with VV-iPDL1/GM at MOI=2 without FBS. Culture media was collected 48 h post infection, and filtered by 0.8 μm syringe filter unit (Millipore, Darmstadt, Germany). The media was incubated with 200 μL Protein G Sepharose (Sigma-Aldrich, St Louis, Mo.) at 4° C. overnight. The protein G beads were washed by 1× PBS three times, and eluted by 0.1 M glycine-HCL, pH=2.8. The elution was dialyzed in 4 L 1× PBS overnight30,31,60. The concentration of the iPDL1 protein was determined using BSA Assay kit (Thermo, Waltham, Mass.).

iPDL1 binding assay by flow cytometry: Tumor cells were infected with PBS, VV-RFP, and VV-iPDL1/GM at MOI=0.5. 24 h later, all cells were collected, and stained with anti-IgG Fc. For some experiments, tumor cells were first cultured for overnight in the presence of IFN-γ (20 ng/mL) to enhance PD-L1 expression and then infected with PBS, VV-RFP, and VV-iPDL1/GM at MOI=0.5. Forty hours later, all the cells were collected, and stained with anti-PD-L1 or anti-IgG Fc. PD-L1+RFP+ (virus-infected) cells and PD-L1+RFP− (uninfected) cells were analyzed by flow cytometry. For detecting purified iPDL1 binding, wild-type MC38 cells and MC38 cells transduced with the recombinant lentiviral vector PD-L1shRNA/GFP were incubated with 50 μg/mL IgG or purified iPDL1 for 30 min on ice. Cells were then stained with anti-PD-L1 or anti-IgG Fc.

Inhibition of PD-1/PD-L1 interaction: Ninety-six-well ELISA plates were coated with 1 μg/well PD-L1 protein (Abcam, ab130039). A total of 50 μL mixture of 20 ng mouse PD-1-biotin (Sino Biological, 50124-M08H-B) and purified iPDL1, IgG control (Sigma, I5381), or anti-PD-L1 antibody control (Biolegend, 124301) at indicated concentration, or 50 μL assay buffer (blank) was added into wells, and incubated at RT for 2 h. Diluted streptavidin-HRP was added to each well after wash and incubated at RT for 1 h with slow shaking. After three times of wash, TMB HRP substrate was added until blue color is developed in the positive control well. OD value at 450 nm UV was measured after 100 μL 2N sulfuric acid was added to stop reaction. (OD of unknown−OD of blank)/(OD of positive control−OD of blank) represents the percent of inhibition activity.

ADCC assay: A total of 1×10⁴/well target cells MC38 or IFNγ-stimulated MC38 cells were seeded in a 96-well plate 1 day before the experiment. On the day of experiment, different amounts of PBS, IgG Fc (Thermo, cat. no.: 31205) or purified iPDL1 were added into wells containing target MC38 cells followed by the addition of 6×10⁴ ADCC bioassay effector cells per well that were provided in the ADCC Reporter Bioassays kit (Promega, Madison, Wis.). After incubation for 6 h at 37° C., the plates were kept on the bench for 15 min. Then each well was added with 75 μL of Bio-Glo Luciferase reagent and kept at RT for 10 min. Luminescence values were measured using a plate reader with glow-type luminescence read capabilities.

Mouse experiments: All the animal experiments were performed in accordance with the guidelines of the Institutional Animal Care and Use Committee of USC, and were bred and maintained in our institute-specific pathogen-free facilities.

B16-F10, B16-F10-Luc, Py230, or MC38 tumors were established by sub-cutaneously injecting 5×10⁵ of corresponding tumor cells into the left flank of C57BL/6J mice (N=5 or 10 per group, the Jackson Laboratory). For the established tumor model, 1×10⁵ B16-F10 cells were injected to the right flank simultaneously. When left flank tumor sizes reached ˜100 mm3 or indicated sizes, tumors were intratumorally injected with 50 μL of the indicated VVs three times on days 0, 3, and 7 (5×10⁷ pfu/tumor), or PBS with or without i.v. injections of 50 μL (200 μg/mL) of anti-PD-Ll antibody. Tumor sizes of treated primary tumors and untreated tumor on the contralateral side (distant tumor) were measured by caliper or monitored by bioluminescence imaging for B16-F10-Luc tumors. Tumor volumes were calculated according to the formula: width²×length×0.5. For tumor rechallenge assay, the treated mice were subcutaneously injected with 2.5×10⁵ B16-F10 cells, 5×10⁵ Py230, or 5×10⁵ MC38 onto the right flank of each mouse at indicated days after the virus treatment. A group of naive mice were injected with tumor cells for control. The rechallenged tumors were monitored as above described. For CD8 T cell depletion experiment, anti-CD8 antibodies (clone: 2.43, Bio X cell, cat. no.: BP0061) were injected i.v. twice weekly starting one day prior to viral injection.

Neoantigen-specific T cell response assays: Splenocytes were isolated from various VV-treated tumor-bearing C57BL/6 mice and cultured in a 96 round bottom well plate (1×10⁵ cells/well) in the presence of single neopeptide or a mixture of neopeptides of MC38 at the indicated concentrations at 37° C. in 5% CO₂. After 80 h incubation, 200 μL supernatants were collected from each well to evaluate IFN-γ via ELISA. [3H] thymidine (1 μCi per well) was added and cultured for an additional 16 h. [3H] thymidine incorporation was measured in TopCount Scintillation and Luminescence Counter. For flow cytometric analysis, splenocytes from the various VV-treated groups were cocultured with syngeneic monocyte-derived DCs (10:1) that were pulsed with neopeptides for 12 h in the presence or absence of Golgi-plug61. Cells were stained with anti-CD8, anti-107a, anti-IFN-γ, anti-IL2, and anti-TNF-α, and analyzed by flow cytometry.

Virus replication assay in vivo: C57BL/6 mice were implanted with 5×10⁵ MC38 cells subcutaneously. When the tumors reached ˜100 mm³, mice were treated with 1×10⁷ pfu/mouse VV-iPDL1/GM intratumorally. On indicated days, mice were killed and tissues were subjected to three cycles of freeze-thaw-sonication to release virus. A total of 500 μL homogenate were incubated on 143B TK cells and titers were determined. Viral titers were standardized to tissue weight.

Generation of neoepitopes-primed T cells: C57BL/6 mice (6-8 weeks) were injected intraperitoneally with a mixture of 11 peptides (10 μg each) formulated with the adjuvant system consisting of 100 μg anti-CD40 (Abclone FJK45) and 100 μg poly (I:C; InvivoGen) two times on days 0 and 14. On day 21, the splenocytes were harvested and in vitro stimulated with irradiated autologous naive splenocytes prepulsed with the peptide mixture for two rounds. Expanded splenic cells were harvested for further experiments.

Isolation of tumor-infiltrating immune cells: C57BL/6 mice were subcutaneously inoculated with MC38 cells (1×10⁶) on one side flank. When the tumor sizes reached ˜100 mm³ or indicated sizes (counted as day 0), mice were intratumorally injected with 50 μL of PBS, VV-RFP, VV-GM, or VV-iPDL1/GM (5×10⁷ pfu/tumor) on days 0 and 3. One group of mice were intraperitoneally injected with 200 μg of anti-PD-L1 antibody (clone 10F.9G2). At indicated days post viral treatment, tumors were collected, weighed, and digested with collagenase type I and DNase for 30 min at 37° C. The tumor tissues were homogenized and then filtered through a 70-μm nylon strainer. Single-cell suspensions were analyzed by FACS or used for other assays.

In vitro and in vivo assays of neoepitopes-specific T cell responses: Tumor-infiltrating DCs from various VV-treated, tumor-bearing mice were isolated using CD11c MicroBeads UltraPure (Miltenyi Biotec, 130-108-338). The DCs were pulsed with indicated neopeptides, and then cocultured with the neoantigens-primed T cells to assess cytokine production and T cell proliferation.

To assess the immunogenicity of the VV-treated tumor cells, MC38 cells seeded in 96-well round bottom plates (5×10³ per well) were infected with PBS, VV-RFP, VV-GM, or VV-iPDL1/GM at MOI=1 for 2 h. Infected MC38 cells were extensively washed and then cocultured with 2×10⁴ the neoantigens-primed

T cells for 48 h: One of mock-infected MC38/CTL cocultures was added with 1 μg/mL anti-PD-L1 antibody. Supernatants were harvested for analyzing IFN-γ production via ELISA. Cells were harvested, immune stained with anti-CD3. T cell numbers were counted by adding precision counting beads (Biolegend, 424902).

To assess in vivo proliferation of the neoantigens-primed T cells in various VV-treated, tumor-bearing mice, MC38 tumor-bearing mice were treated with the indicated viruses (5×10⁷ pfu), followed by adoptive transfer of CF SE-labeled 2×10⁶ the neoantigens-primed T cells. Three days later, tumor-draining lymph nodes (TdLNs) were harvested and their proliferation was assessed based on CFSE dilution via flow cytometry. Data shown are a representative histogram of two independent experiments.

CTL assay: Firefly Luciferase stably expressing cells were cocultured with effector T cells at the indicated ratios. Fortyeight hours later, all the cells were spun down and resuspended in 100 μL media supplemented with 100 μg/mL Beetle Luciferin Potassium Salt and incubated at RT for 5 min. Cells were transferred to 96-well white opaque plate. Luciferase emission was measured on a TopCount Scintillation and Luminescence Counter. Killing lysis %=[1−(unknown−blank)/(positive control−blank)]×100%.

RNA sequencing: MC38 cells were infected with VV-iPDL1/GM. Cells were harvested at various times. Cellular RNAs were extracted from cell lysates using RNeasy Plus Mini Kit (Qiagen). Total RNA is enriched by oligo (dT) magnetic beads (rRNA removed). RNA-seq library preparation using KAPA Stranded RNA-Seq Library Prep Kit (Illumina). The libraries were sequenced on a HiSeq 4000 instrument using 2×150 bp pair-end sequencing (Arraystar Inc, Rockville, Md.).

Software: Odyssey v3.0, MikroWin2000, Living Image v4.4, FACS DIVA 6.1.2, Illustrator CS6, flowjo 10.4.0, Graphpad prism 6, Microsoft excel 2011 for mac, Living Image v4.3.1, RNA-seq analysis was performed with the following software HTSeq v0.5.3, Solexa pipeline v1.8, FastQC software 0.11.7, Hisat2 software, StringTie 1.3.3, R 3.4.1, and Python 2.7.

Statistics: Statistical analysis was performed using GraphPad Prism 6. When passing the normality test, two-tailed Student's t-test was used to compare the two groups. Otherwise, a Mann-Whitney U test was used. Repeated-measures two-way ANOVA with Bonferroni's correction was used to compare the effect of multiple levels of two factors with multiple observations at each level (for tumor volumes). Animal survival is presented using Kaplan-Meier survival curves and was statis-tically analyzed using log rank test. The data presented in the figures are mean±SD. P values<0.05 were considered to statistically significant.

The RNA-seq data have been deposited in the NCBI GEO database under the accession code GSE145823.

### Example 2

### Preclinical Study of an Engineered Oncolytic Vaccinia Virus Co-Expressing a Human PD-L1 Inhibitor and Human GM-CSF

### 2.1 Generation and Characterization of a Recombinant Oncolytic Vaccinia Virus Coexpressing Human PD-L1 Inhibitor and GM-CSF (VV-ihPDL1/GM).

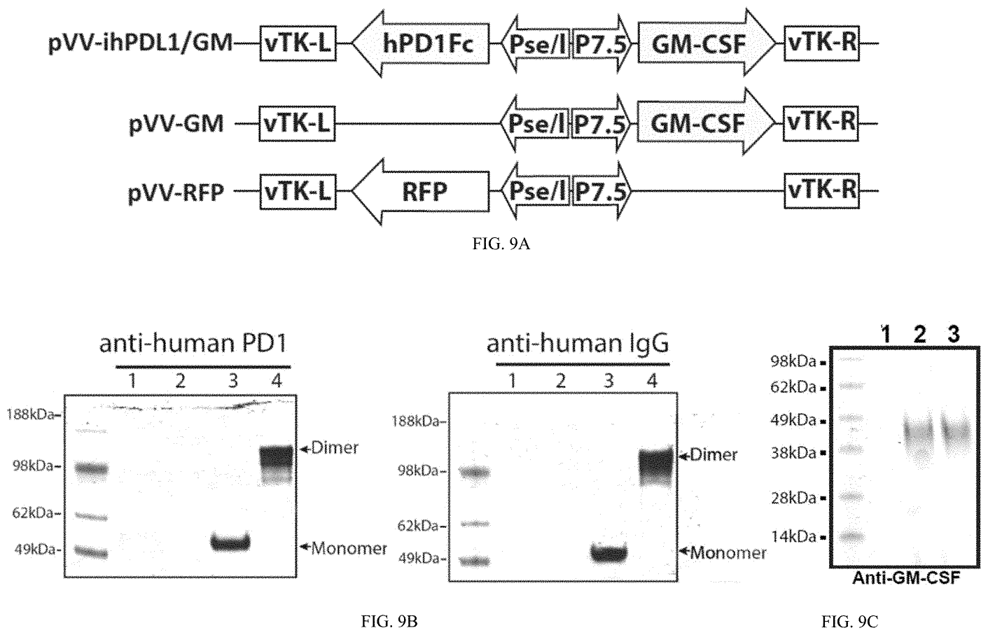

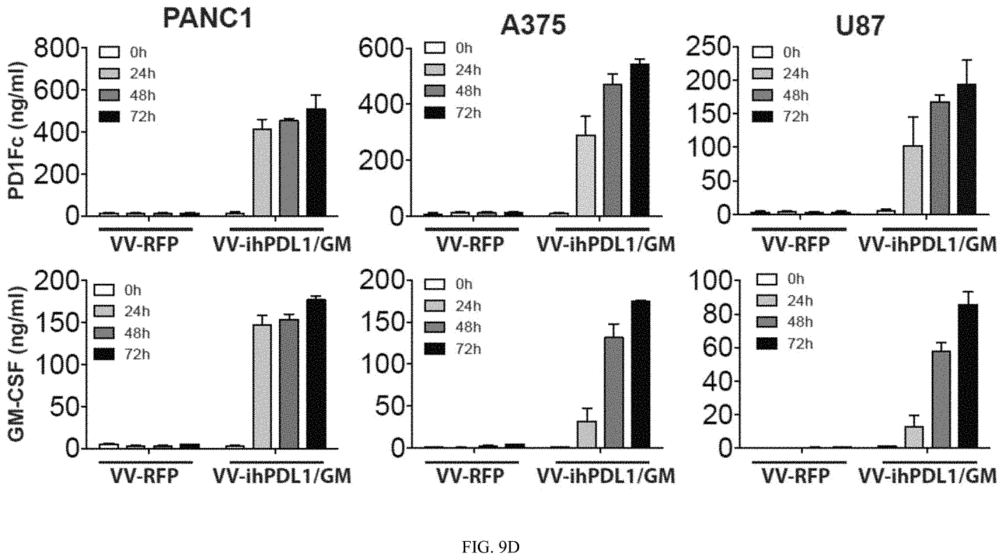

A recombinant vaccinia virus shuttle vector pVV-ihPDL1Fc/GM that coexpresses human PD1-Fc fusion protein (ihPDL1) controlled by the vaccinia virus Pse/1 promoter or/and human GM-CSF by the vaccinia virus p7.5 later early promoter was constructed. Control shuttle vectors, pVV-RFP that only expresses RFP marker controlled by the vaccinia virus Pse/1 promoter and pVV-GM that only expresses human GM-CSF, were also constructed (FIG. 1A). CV-1 cells were infected with vaccinia virus growth factor (vgf)-deficient vaccinia virus vSC20 and then co-transfected with one of the recombinant shuttle plasmids for homologous recombination. PCR assays were performed to select the recombinant viruses that have the transgenes at the tk locus of vaccinia viral genome. Once DNA sequencing confirmed these recombinant VVs, the viruses were used to infect H226 tumor cells (squamous cell carcinoma) and analyzed for expression of the inserted genes by Western Blot. Result showed that both αPDL1Fc and GM-CSF were expressed and secreted as manifested by the bands corresponding to the monomer or dimer of ihPDL1 detected by anti-IgG and the band corresponding to GM-CSF detected by anti-GM-CSF from the supernatants shortly after infection (FIGS. 9B, 9C). To further analyze the VV-infection of tumor cells, several human tumor cell lines including PANC1 (pancreatic cancer cell line), U87 (glioblastoma), H226 (squamous cell carcinoma), and A375 (melanoma) were infected with the VVs. At various times, the supernatants of the viral-infected cells were harvested and analyzed via ELISA. Results showed that the tumor cell lines expressed and secreted ihPDL1 or/and GM-CSF (FIG. 9D).

### 2.2 VV-ihPDL1/GM Retains the Ability to Preferentially Replicate and Kill Human Tumor Cells.

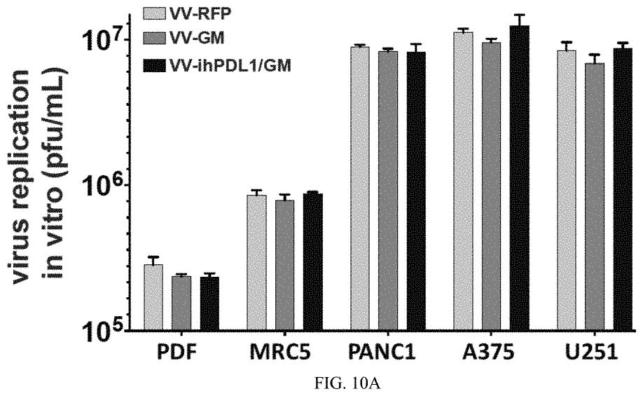

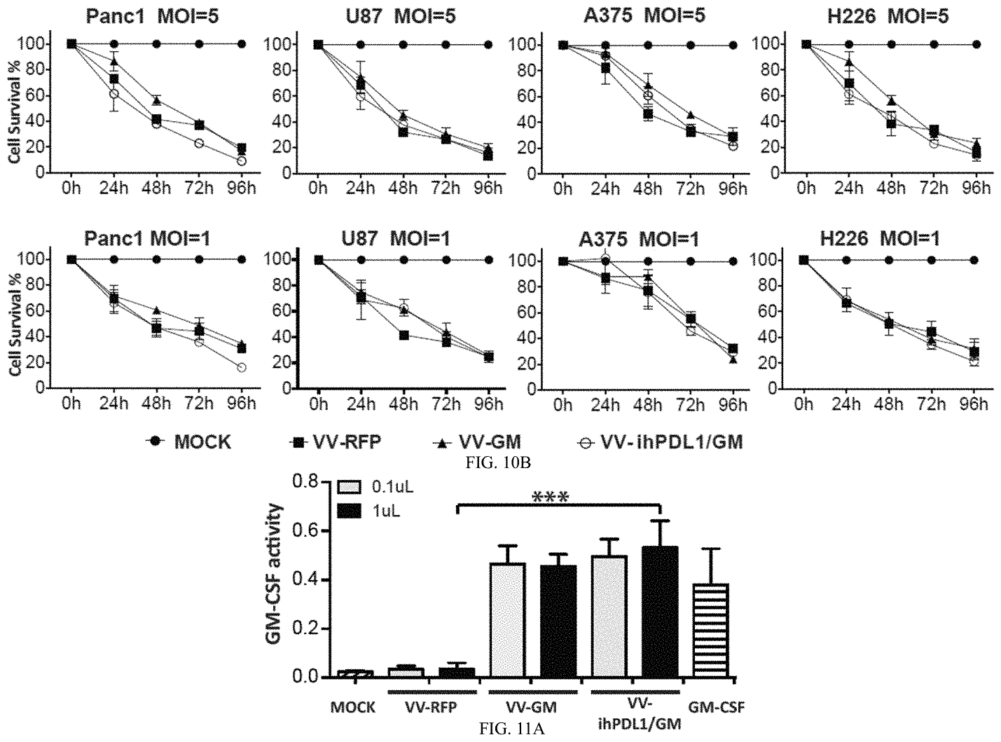

FIG. 10A shows that VV-ihPDL1/GM preferentially replicates in human tumor cells. 2×10⁵ normal cells or tumor cells as indicated were infected with VV-RFP, VV-GM, or VV-ihPDL1/GM at a low dosage (MOI=0.5) for 24 h, 48 h, 72 h. Infected cells were harvested, and frozen/thawed three times to release viral particles in 1mL media. The viral particles were titrated as described in material and methods. Experiment was repeated twice.

FIG. 10B shows the oncolytic activity of VV-ihPDL1/GM against various types of human tumor cells. Human tumor cell lines (Panc1, U87, A375, or H226) were infected with the indicated VVs at a MOI of 5 or 1 for 24, 48, 72, and 96 hrs. MTT assay were performed to determine viability of different infected tumor cells. The cell survival percentage is expressed as the viability of different viral-infected cells relative to that of mock-infected cells at the time point. Data are presented as means±SD. Experiments were repeated twice.

2.3 GM-CSF Secreted from Infected Tumor Cells Promotes Dendritic Cell (DC) Differentiation.

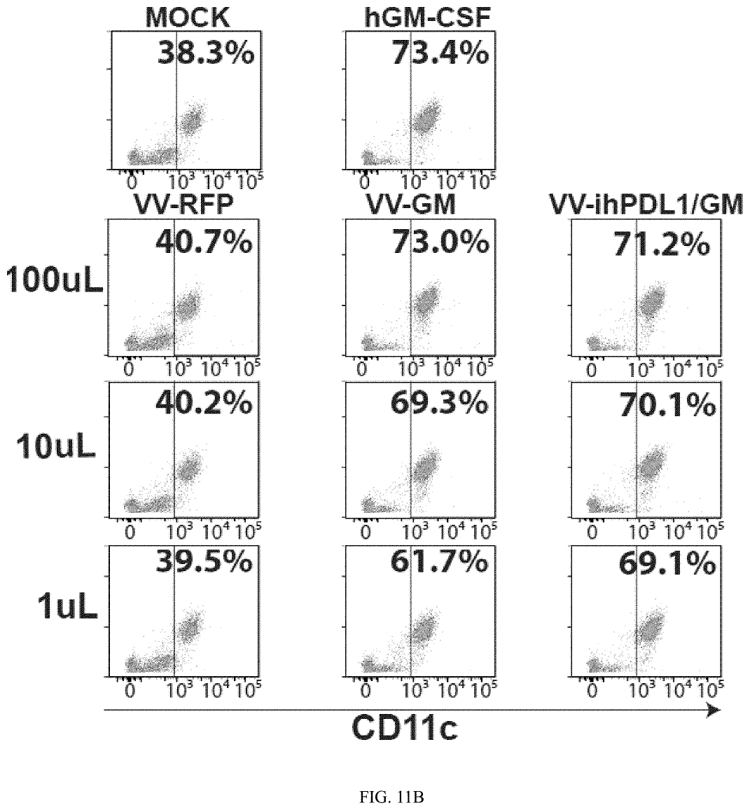

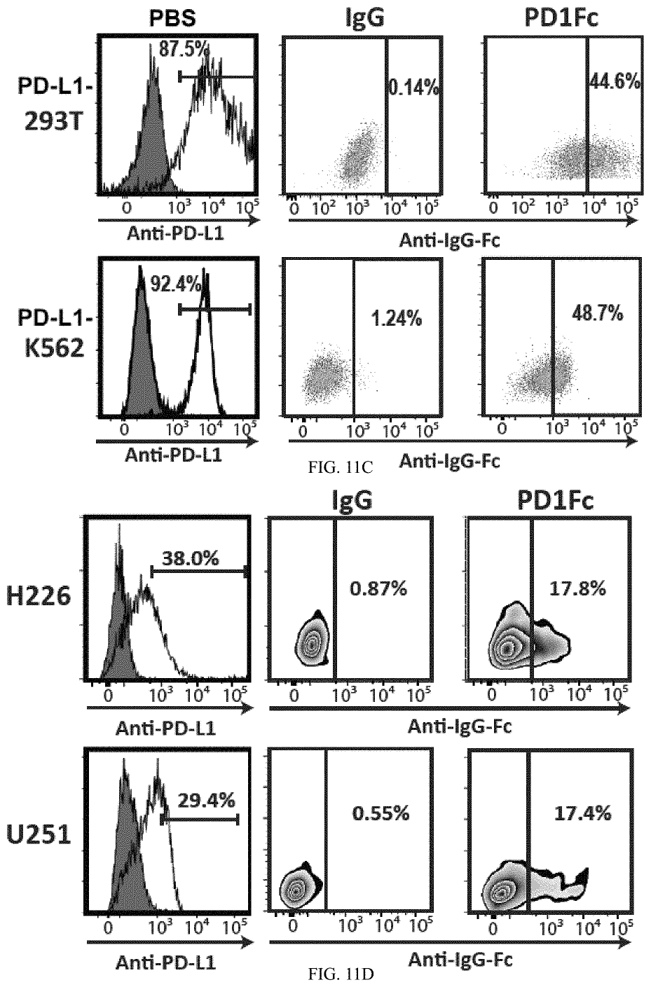

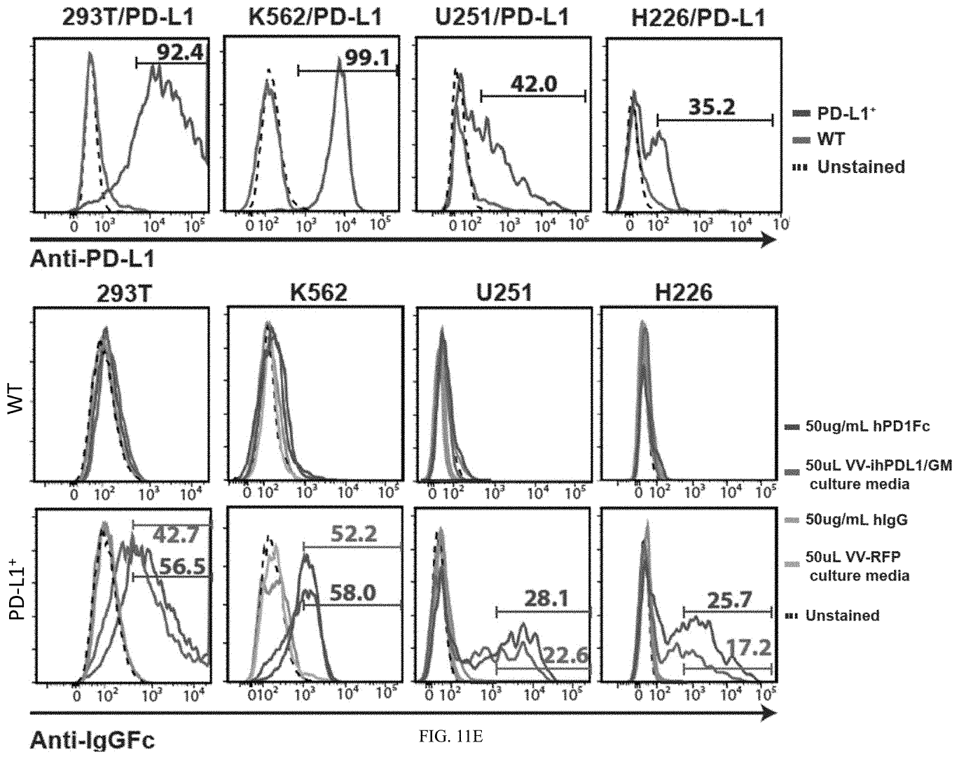

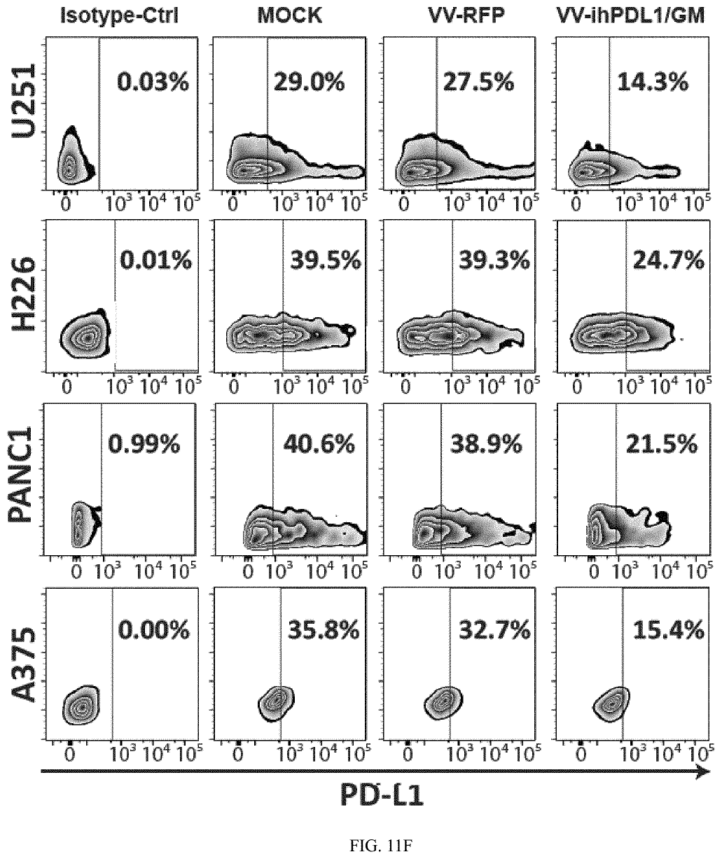

TF-1 cell is a factor-dependent human erythroleulemia cell line. TF-1 cell proliferation assay is widely employed for quality assurance and quality control of GM-CSF. To confirm biological function of GM-CSF secreted from the infected tumor cells, PANC1 tumor cells were infected with VV-ihPDL1/GM, VV-RFP, or VV-GM for 48 h. Supernatant was collected and filtered through a 0.22-um inorganic membrane filter to remove VV particles (with an average size 360 nm×270 nm×250 nm). Various volumes of the filtered supernatant were applied onto the TF-1 cell cultures. MTT assay analyzing the TF-1 cell proliferation showed that addition with 0.1 μL of the supernatant from VV-GM- or VV-ihPDL1/GM-infected PANC1 cells reached an equivalent effect to that achieved by adding a commercial GM-CSF (2 ng/ml) in support of TF1 growth (FIG. 11A).

Furthermore, we directly analyzed impact of the secreted GM-CSF on DC differentiation. Monocytes derived from healthy PBMCs were cultured in complete RPMI-1640 media supplemented with commercial GM-CSF and IL-4 for 3 days. Non-adherent or loosely adherent cells were collected and cultured in complete RPMI-1640 media supplemented with commercial IL-4 (100 ng/mL) and various volumes of the filtered supernatant of VV-infected PANC1 cells for 48 h. All the cells were collected and analyzed for CD11c expression via immune staining and flow cytometry (FACS). Result showed that addition with as low as 1 μL of the supernatant form VV-GM or VV-ihPDL1/GM-infected PANC1 cells, not from VV-RFP infected PANC1 cells, reached a comparable effect on addition with the commercial GM-CSF (50 ng/mL) in support of CD11c⁺ DC differentiation (FIG. 11B). These studies demonstrated that secreted GM-CSF is biologically active in support of CD11c⁺ DC differentiation.

2.4 ihPDL1 Secreted from Infected Tumor Cells Binds to PD-L1+ Tumor Cells.

PD-1 checkpoint therapy using anti-PD-1 or anti-PD-L1 achieved significant clinic successes in treating a variety of cancer patients by activating their own effector T cells. To test if the secreted ihPDL1 has a potential to block PD-1/PD-L1 checkpoint pathway, we first examined if ihPDL1 is capable of binding PD-L1 expressed on cell surfaces. ihPDL1 was purified from supernatant of VV-αPDL1/GM-infected PANC1 cells using Protein A/G beads. Purified ihPDL1 were incubated with PD-L1-transduced 293T or K562 cells (FIG. 11C) or IFN-γ pre-stimulated H226 or U251 tumor cells (FIG. 11D). A conjugated anti-IgG-Fc was used to detect the binding of ihPDL1 to PD-L1 on these cell surfaces via flow cytometry. Result showed that purified ihPDL1 bound to PD-L1⁺ tumor cells, as detectable by the conjugated anti-IgG-Fc (FIGS. 11C&11D). The supernatants containing secreted ihPDL1 of VV-αPDL1/GM-infected tumor cells also showed the binding to PD-L1⁺ tumor cells (FIG. 11E). Furthermore, the supernatants containing secreted ihPDL1 of VV-αPDL1/GM-infected tumor cells blocked the engagement between surface PD-L1 and anti-PD-L1 antibody, implying an interaction of ihPDL1 and PD-L1 occurring on the tumor cell surfaces (FIG. 11F).

2.5 ihPDL1 Secreted from Infected Tumor Cells Inhibits PD-1/PD-L1 Interaction and has ADCC Activity.

The binding affinities of purified ihPDL1 to PD-L1 molecules were further compared with commercial anti-PD-L1 antibody by Surface Plasmon Resonance (SPR) analysis. The result of which demonstrated that both ihPDL1 and anti-PD-L1 specifically bind to PD-L1 however with a little different pattern (FIG. 12A).

After demonstrating ihPDL1 interaction with PD-L1 expressed on tumor cell surfaces, we performed a competitive inhibition assay to investigate if ihPDL1 could block PD-1/PD-L1 binding that is essential for triggering PD-1/PD-L1 checkpoint signaling. Various dosages of purified ihPDL1 or the sera from VV-ihPDL1/GM-treated mice were mixed with a commercial biotin-PD1 (BPS Bioscience, San Diego, Calif.) and added into each well of a PD-L1-coated microplate. ELISA analyses showed that at the tested concentrations, ihPDL1 had an equivalent activity to the commercial anti-PD-L1 in blocking the biotin-PD1 binding to the PD-L1-coated wells (FIG. 12B), indicating that ihPDL1 inhibits PD-1/PD-L1 interaction.

Given that blockade of PD-1/PD-L1 interaction enhances activation of effector T cells, we tested if addition of ihPDL1 can promote T cell activity in a Mixed Lymphocyte Reaction (MLR) assay. T-cells were isolated from healthy PBMCs and co-cultured with irradiated (2500 rads) allogeneic mature DCs at a ratio of 10:1 in the presence of isotype IgG, purified ihPDL1 or the sera from VV-ihPDL1/GM-treated mice, or commercial anti-PD-L1 for 3 days. T cell activation was measured by examining the IFN-γ level in supernatants of the mixed cultures via ELISA. Result showed that at the tested concentrations (0.01 μg/mL, 0.1 μg/mL, or 1 μg/mL) of the purified ihPDL1 displayed an equivalent activity to the commercial anti-PD-L1 (FIG. 12C).

The Fc fragment in ihPDL1 potentially confers ihPDL1 with an ADCC activity by engaging FcγRIII (CD16) expressing on natural killer (NK) cells that is important for antibody mediating cytotoxicity against tumor. To test if ihPDL1 is capable of mediating an ADCC, serial dilutions of human IgG, purified ihPDL1, or Mock (PBS) were incubated with Jurkat effector cells (Promega ADCC Bioassay Effector cells) and K562/PD-L1 or IFN-γ-stimulated U251 (U251/PD-L1) or H226 (H226/PD-L1) target cells. ihPDL1-mediated ADCC reaction was quantified through measuring luciferase (luc) produced in the Jurkat effector cells. Result showed that addition with as low as 10 ng/mL of ihPDL1 significantly enhanced luc production in the Jurkat effector cells no matter which PD-L1-expressed tumor cell line used as targets (FIG. 12D), implying that ihPDL1 has a potential to mediate an ADCC reaction.

2.6 High Levels of Serum ihPDL1 and GM-CSF in Tumor-Bearing Mice Treated with VV-ihPDL1/GM.

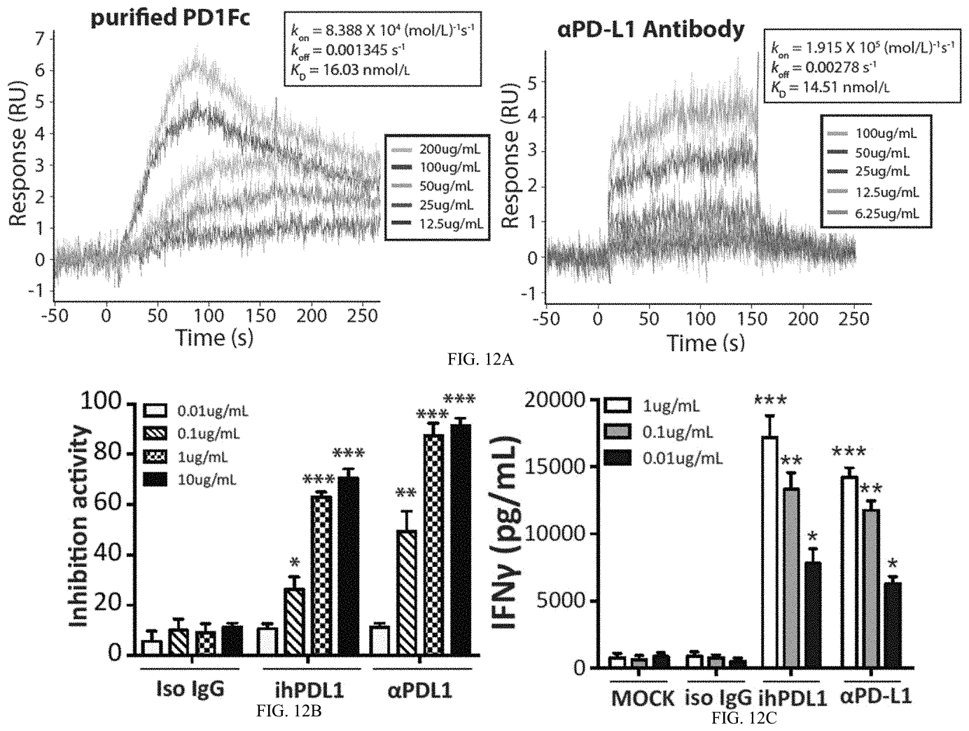

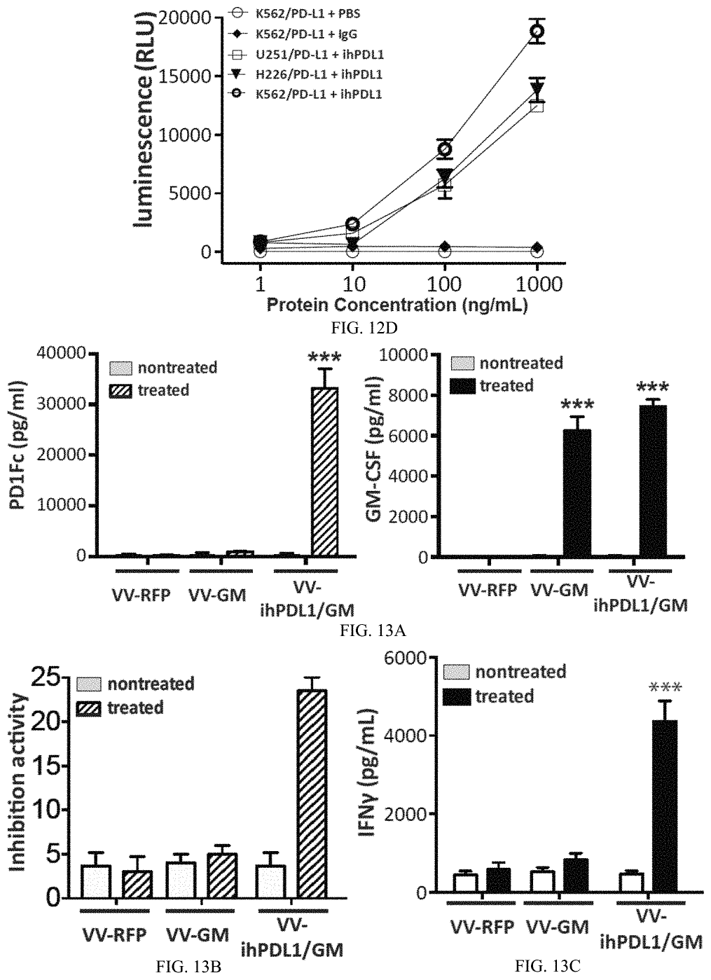

H226 cells were subcutaneously inoculated into one side flank of NSG mice. When the median tumor volume reached 100 mm³, groups of tumor-bearing mice were injected intratumorally with 1×10⁸ pfu of VV-RFP, VV-GM, VV-ihPDL1/GM. Prior to the viral injection and 48 h post-viral injection, mice were bled for measuring serum ihPDL1 and GM-CSF levels via ELISA. High levels of serum iPDL1 and GM-CSF were detected in tumor-bearing mice after intratumor injection of VV-ihPDL1/GM (FIG. 13A).

2.7 Sera of Tumor-Bearing Mice Treated with VV-ihPDL1/GM ihPDL1 are Able to Inhibits PD-1/PD-L1 Interaction.

FIG. 13B shows that VV-ihPDL1/GM-treated mouse sera inhibited PD-1/PD-L1 interaction. PD1-biotin (10 ng) was mixed with 100 μL of different virus treated mouse sera in a volume of 200 μL. The mixture was then added into PD-L1-coated 96-well plate. After incubation at RT for 2 h, diluted streptavidin-HRP was added followed by addition with TMB substrate. The inhibition activity was expressed as (OD450 of MOCK−OD450 of sera)/(OD450 of MOCK−OD450 of background)×100%. Data are presented as means±SD. p<0.01, or VV-ihPDL1/GM vs. VV-RFP -treated mice. The experiment was triplicated with similar results.

FIG. 13C shows that VV-ihPDL1/GM-treated mouse sera enhanced T cell activity in MLR assay. T-cells isolated from healthy PBMCs were co-cultured with irradiated allogeneic mature DCs at a ratio of 10:1 for 5 days in the presence of 100 μL different virus treated mouse sera in a volume of 200 μL. IFN-γ level in the media was measured via ELISA. Data are presented as means±SD. p<0.05, or VV-ihPDL1/GM vs. VV-RFP-treated. The experiment was triplicated with similar results.

### 2.8 VV-ihPDL1/GM-Treated Mouse Sera Enhanced the Cytolytic Activity of CAR-T Cells Against PD-L1⁺ Tumor Cells.

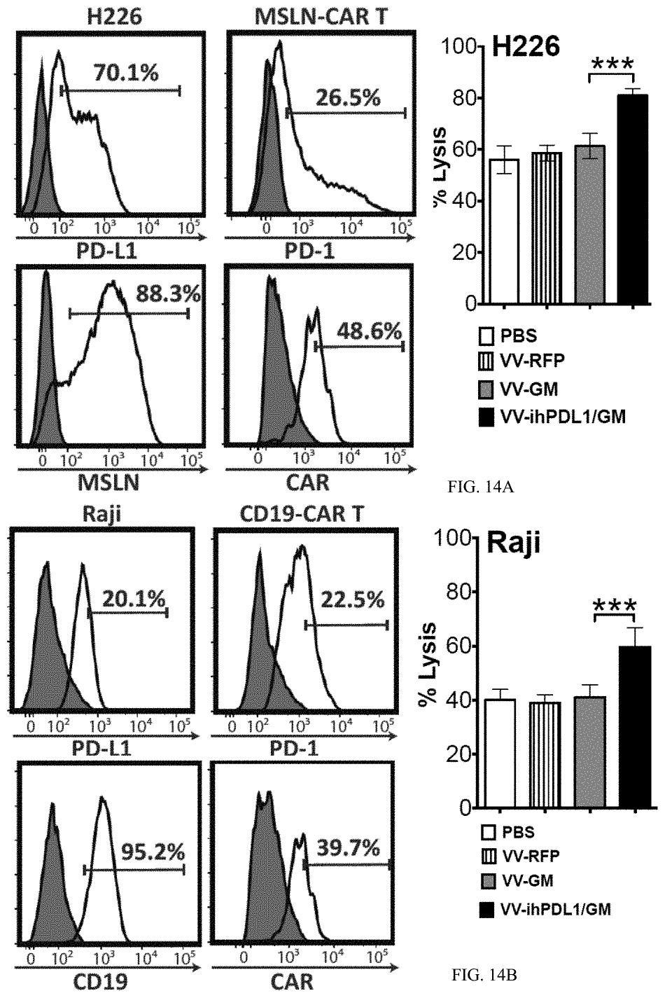

Chimeric antigen receptor T cells (CAR T) anti-tumor therapy shows very potent activities against leukemia and lymphoma; however, their efficacy against solid tumors are limited, probably due to the poor T cell filtration and the expression of checkpoint inhibition such as PD-L1 on tumor cells. To test if mouse sera contain a sufficient level of ihPDL1 to enhance the cytolytic activity against PD-L1+ tumor, mesothelin (MSLN)-targeted CAR-T cells (FIG. 14A) were co-cultured with H226 tumor cells transduced with MSLN and PD-L1 (E:T=10:1) or Raji cells (E:T=5:1) in the presence of 25 μL different VV-ihPDL1/GM-treated mouse sera for 48 h. Killing activity of MSLN-CAR T cells against target tumor cells was measured by luc-based CTL assay (Promega). It was found that ihPDL1/GM-treated mouse sera significantly enhanced the cytotoxicity of MSLN-CAR-T cells against PD-L1⁺MSLN⁺ tumor cells (FIG. 14A).

Moreover, CD19-targeted CAR-T cells were co-cultured with PD-L1⁺CD19⁺ Raji cells (E:T=5:1) in the presence of 25 μL different VV-ihPDL1/GM-treated mouse sera for 48 h. ihPDL1/GM-treated mouse sera significantly enhanced the cytotoxicity of CD19-CAR-T cells against PD-L1⁺CD19⁺ tumor cells (FIG. 14B).

### 2.9 Materials and Techniques

Cell lines: Monkey kidney cell line CV1, human embryonic kidney cell line 293T, osteosarcoma HuTK-143B, human primary dermal fibroblast cell line PDF, human lung fibroblast cell line MRC5, and a few of human tumor cell lines including pancreas carcinoma PANC1, glioblastoma U87 and U251, lung squamous carcinoma H226, and malignant melanoma A375, etc. were purchased from ATCC and grown in Dulbecco's modified Eagle's medium (DMEM) and human primary cells were culture in complete RPMI medium both supplemented with 10% fetal bovine serum (FBS), 2 mM L-glutamine, and 1× penicillin/streptomycin solution (Invitrogen, Carlsbad, Calif.) in 37° C., 5% CO₂ incubator. All stable cell lines were lentivirally transduced to express firefly luciferase and PD-L1. The transduced cell lines were either GFF or anti-PD-L sorted using BD FACS Aria cell sorter II (BD Bioscience, USA), or selected by puromycin.

Recombinant VVs: Vaccinia virus shuttle vector pSel-DsRed2N1 was used to construct the recombinant shuttle vectors pVV-ihPDL1-GMCSF, and pVV-GMCSF with expression of ihPDL1 fusion protein controlled by the vaccinia virus Pse/1 promoter and GM-CSF by the vaccinia virus p7.5 later early promoter. A control shuttle vector (pVV-RFP) only expresses RFP (red fluorescense protein) marker controlled by the vaccinia virus Pse/1 promoter. To generate recombinant VVs, a vgf gene-deleted WR strain VV, vSC20, was used as a parental virus for homologous recombination. CV-1 cells were infected with vSC20 at multiplicity of infection (MOI) of 0.1 and then transfected with one of the recombinant shuttle plasmids. Selection of the recombinant viruses was based on PCR assays that confirm recombination of the foreign genes into the tk locus of vaccinia viral genome.

Replication and oncolytic activity of VV: Normal cells or different tumor cells were infected with VV-RFP, VV-GM, or VV-ihPDL1/GM at various MOIs for 24 h, 48 h, 72 h, or 96 h. For determining replication, infected cells were harvest, and frozen/thawed three times to release viral particles for titration. For determining oncolytic activity, MTT assay were performed to determine viability of different infected tumor cells.

Titration of viruses: HuTK-143B cells (2×10⁵) were seeded into 12 well plates for 24 h. Different VVs with 10-fold serial dilutions were added onto the cell monolayer. After 1 h incubation with rocking, the cells were gently added with 2 mL culture media and incubated for 24-48 h. The cells were washed and fixed in 0.1% crystal violet in 20% ethanol. The plaques were counted under microscope.

Western Blot: CV1 cells were infected with recombinant VV at MOI=2.0 for 48 hours. Supernatant were harvested and clarified. Samples were electrophoresed in a 4-20% gradient sodium dodecyl sulfate-polyacrylamide gel and transferred onto a 0.22-μm nitrocellulose membrane (Hybond, Amersham Biosciences, Sunnyvale, Calif.). Immunodetection was performed with anti-human IgG using IRDYE® 800CW Goat anti-Human IgG (H+L) (Licor, cat. 925-32232), or mouse anti-human PD-1 antibody (Biolegend, Cat: 367402), or anti-GM-CSF (clone: BVD2-21C11, Biolegend). The blots were detected with an Odyssey Imager (LI-CON, Lincoln, Nebr.).

ELISA: GM-CSF was detected using GM-CSF ELISA kit (Biolegend, Cat: 432004). ihPDL1 was detected using human PD-1 DuoSet ELISA (biolegend, Cat: DY1086)

ihPDL1 purification: 1×10⁷ HuTK-143B cells were infected with VV-ihPDL1/GM at MOI=5. 2 h later when the infection was done, media with viruses was replaced by DMEM without FBS. 48 h post-infection, supernatants were harvested and filtered by 0.45 μm syringe filter unit (Millipore, Darmstadt, Germany). The media was incubated with 500 μL Protein G Sepharose (Sigma-Aldrich, St Louis, Mo.) at 4° C. overnight with gentle shaking. After three times' wash with 1× PBS, the G protein beads were eluted by 0.1 M glycine-HCL, pH=2.8 followed by twice dialysis in 4 L 1× PBS overnight each time. The concentration of the dialyzed purified ihPDL1 protein was determined by BSA Assay kit (Thermo, Waltham, Mass.).

TF-1 cell assay: TF-1 cells are a GM-CSF or IL-3-dependent human erythroleukemic cell line. To test activity of the secreted GM-CSF, TF-1 cells cultured in a 96-well plate in the presence of various dosages of the filtered supernatants from different VV-infected tumor cells or with 2 ng/ml of commercial GM-CSF as a positive control. MTT assay (Promega) was used to assess proliferation of TF-1 cells under different conditions.

Human dendritic cell (DC) differentiation assay: Monocytes derived from healthy PBMCs were cultured in complete RPMI1640 media supplemented with 50 ng/mL GM-CSF and 100 ng/mL IL-4 for 3 days. All non-adherent or loosely adherent cells were collected, resuspended in complete RPMI1640 media supplemented with 100 ng/mL IL-4, and aliquoted into a 12-well tissue plate. The cultured cells were added with various doses of the filter (0.1 μm)-treated culture supernatants of tumor cells (PANC1) infected with different viruses or with 50 ng/mL commercial GM-CSF for positive controls. All the cells were incubated for another 48 h and then collected for CD11c staining and flow cytometry.

Surface plasmon resonance (SPR)-binding assays: The assays were carried out at 25° C. using a Biacore T100 (USC NanoBiophysics Center). CMS sensor chips were activated by a 7 min injection of a 1:1 mixture of N-hydroxysuccinimide and 1-ethyl-3-(3-dimethylaminopropyl) carbodiimide hydrochloride at a flow rate of 10 μL/min. The PD-L1-His tag protein was prepared at a concentration of 10 μg/mL in 10 mM acetate buffer, pH 4.0, and immobilized to the sensor chips by amine linkage, with the typical immobilization levels being 100 response units. For blocking sensor chips, 70 μL of 1M ethanolamine-HCl buffer, pH 8.5, was injected and the chips were further washed with 10 μL of 10 mM glycine-HCl buffer, pH 1.5. For analyzing the binding and equilibrium affinity of ihPDL1 or a-PD-L1 antibody (biolegend, cat 329702), 5 serial dilutions of ihPDL1 starting from 200 μg/mL, or 6 serial dilutions of α-PD-L1 antibody starting from 100 μg/mL were injected at a flow rate of 30 μL/min over the sensor chips coupled with PD-L1 protein. The data sets were analyzed using a model for 1:1 binding.

ihPDL1 Binding assay: 24 h IFN-γ stimulated U251 or H226 cells were incubated with various doses of purified ihPDL1, concentrated supernatants of VV-infected cells, or commercial human IgG (sigma, St Louis, Mo.) for 30 min on ice. Cells were washed twice, and followed by staining of viability and anti-IgG-Fc (Biolegend, San Diego, Calif.). Cells stained with anti-PD-L1 (Biolegend, San Diego, Calif.) served for a positive control. The stained cells were analyzed by flow cytometry.

Inhibition of PD1/PDL1 interaction assay: Microplates were coated with 1 μg/well commercial PD-L1 protein (BPS Bioscience, San Diego, Calif.). 50 μL mixture of 10 ng PD-1-biotin with Mock, IgG, purified ihPDL1, or commercial anti-PD-L1 at the various concentrations was added into each coated well. The plate was incubated at room temperature (RT) for 2 h. Diluted streptavidin-HRP was added to each well after wash. The plate was incubated at RT for 1 hour with slow shaking. After 5-time washes, 100 μL TMB HRP substrate (Thermofisher) was added and the plate left at RT until blue color is developed in the positive control well.

Mixed lymphocyte reaction (MLR): T-cells were isolated from healthy PBMCs and co-cultured with irradiated (2500 rads) allogeneic mature DCs at a ratio of T: DC =10:1 in the presence of isotype IgG, purified ihPDL1, mouse sera or commercial anti-PD-L1 at the various concentrations for 5 days. IFN-γ levels in the media were measured via ELISA.

ADCC assay: Purified ihPDL1 was analyzed with ADCC kit according to the manufacturer's instruction (Promega, Madison, Wis.).

Flow cytometry: Flow cytometry was used to ihPDL1 binding, DC differentiation, T cell stimulation, PD-L1 and CAR expression of cells in vitro and in vivo, as well as immune cells population in treated or distant tumor microenvironment. The following antibodies were used in flow cytometry experiments: anti-tEGFR antibody (cetuximab human IgG1, Absolute Antibodies Ltd., Oxford, UK), anti-PD-L1 (PE, clone: 29E.2A3, Biolegend), anti-IgG-Fc (Polyclonal, Thermo), anti-CD11c (PE, or APC, clone: 3.9, BD Biosciences), Viability dye (BV510 or UV450, Tonbo Biosciences), anti-CD19 (FITC, clone:4G7, Biolegend), anti-mesothelin (PE Clone: 420211, E&D systems).

Cytotoxicity assay: Effector T cells were mixed with luciferase (luc)-expressed target cells at various ratios and seeded into 96-well plates and cocultured for 48 h. Luc activity was measured using a luciferase assay system (Promega). CTL activity=[OD of (Mock+Target)−OD of (Effector+Target)HOD of (Mock+Target)−OD of background]×100%.

Statistical analyses: All data are presented as means and standard errors (s.e.). Analysis of variance was used to determine the level of differences between groups. Different groups were compared using the Student-Newman-Keuls test with SigmaStat 2.03 software (SPSS, Inc.), or a Chi-square test (be indicated in the text). P values were considered significant at <0.05.

Software: Odyssey v3.0, MikroWin2000, FACS DIVA 6.1.2, Illustrator CS6, flowjo 10.4.0, Graphpad prism 6, Microsoft excel 2011 for mac.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A composition comprising a recombinant oncolytic virus, wherein the recombinant oncolytic virus comprises one or more nucleic acid sequences encoding
a polypeptide inhibitor of programmed death-ligand 1 (PD-L1) or a polypeptide inhibitor of programmed cell death protein 1 (PD-1), and
granulocyte-macrophage colony-stimulating factor (GM-CSF).

2. The composition of claim 1, wherein the recombinant oncolytic virus comprises vaccinia virus, herpes simplex virus, reovirus, vesicular stomatitis virus, poliovirus, senecavirus, or Semliki Forest virus.

3. The composition of claim 2, wherein the recombinant oncolytic virus comprises vaccinia virus, wherein the vaccinia virus has both thymidine kinase and vaccinia growth factor viral gene deleted from its backbone or inactivated.

4. The composition of claim 1, wherein the one or more nucleic acid sequences encode, in expressible form, the polypeptide inhibitor of PD-L1 and the GM-CSF, wherein the polypeptide inhibitor of PD-L1 is a fusion protein comprising an extracellular domain of programmed cell death protein 1 (PD-1) and a crystallizable fragment of immunoglobulin class G (IgG-Fc).

5. The composition of claim 4, wherein the one or more nucleic acid sequences encode a polypeptide inhibitor of human PD-L1 and human GM-CSF, and
wherein the one or more nucleic acid sequences comprises a first nucleic acid sequence of SEQ ID NO:51 and a second nucleic acid sequence of SEQ ID NO:53, or the polypeptide inhibitor of human PD-L1 has an amino acid sequence of SEQ ID NO:52 and the human GM-CSF has an amino acid sequence of SEQ ID NO:54.

6. The composition of claim 1, wherein the recombinant oncolytic virus induces apoptosis of a tumor cell resistant to GM-CSF, resistant to an oncolytic virus without a nucleic acid sequence encoding any of the polypeptide inhibitor of PD-L1, the polypeptide inhibitor of PD-1, and the GM-CSF, or resistant to both the GM-CSF and the oncolytic virus with the nucleic acid sequence.

7. A system, comprising the composition of claim 1 and a mammalian cell, wherein upon infecting the mammalian cell with the composition of claim 1, the mammalian cell secretes the polypeptide inhibitor encoded by the one or more nucleic acid sequences of the recombinant oncolytic virus of the composition of claim 1.

8. A composition comprising serum isolated from a mammal infected with a recombinant oncolytic virus, wherein the recombinant oncolytic comprises one or more nucleic acid sequences encoding
a polypeptide inhibitor of programmed death-ligand 1 (PD-L1) or a polypeptide inhibitor of programmed cell death protein 1 (PD-1), and
granulocyte-macrophage colony-stimulating factor (GM-CSF),
wherein the serum contains the polypeptide inhibitor of PD-L1 or the polypeptide inhibitor of PD-1.

9. A method of treating a subject suffering from cancer, comprising:
administering to the subject an effective amount of the composition of claim 1 to induce infiltration of one or more T cells into the cancer.

10. The method of claim 9, wherein the cancer comprises adenoma, melanoma, neoplasm of mammary, pancreatic cancer, glioblastoma, colorectal cancer, or a combination thereof.

11. The method of claim 9, wherein the composition is administered intratumorally or the composition is delivered into the tumor.

12. The method of claim 10, which is effective for inhibiting the growth or reducing the size of the tumor into which the composition was administered or delivered, and further effective for inhibiting the growth or reducing the size of a distant tumor in the subject.

13. The method of claim 9, wherein the subject has an existing tumor or a reoccurring tumor.

14. The method of claim 9, further comprising administering to the subject additional agent selected from an inhibitor of PD-1, an inhibitor of PD-L1, a chemotherapeutic agent, or a combination thereof.

15. The method of claim 13, wherein the subject's splenic T cells are responsive to tumor neoantigens at 10 days, 20 days, 30 days, 40 days, or longer, after the administration.

16. The method of claim 13, wherein the subject's response to a therapy consisting essentially of an inhibitor of PD-1, an inhibitor of PD-L1, or both is ineffective.

17. The method of claim 9, resulting in tumor-infiltrated T cells, and the method further comprises isolating the tumor-infiltrated T cells from the cancer of the subject, expanding the tumor-infiltrated T cells ex vivo, and transferring the expanded tumor-infiltrated T cells to the subject suffering from the cancer.

18. The method of claim 9, wherein the subject has recovered from a cancer, and the method is effective is promoting immune response in the subject against tumor relapse.

19. A method for generating tumor-infiltrating, oncolytic virus-induced T cells, comprising:
administering, to a subject having a cancer, an effective amount of the composition comprising the recombinant oncolytic virus of claim 1, to induce infiltration of one or more T cells into the cancer, resulting in tumor-infiltrated T cells;
isolating the tumor-infiltrated T cells from the cancer of the subject; and optionally expanding the tumor-infiltrated T cells ex vivo.

20. A method of treating or reducing severity of cancer in a subject, comprising administering to the subject an effective amount of a composition comprising the serum according to claim 8 to induce infiltration of one or more T cells in the cancer, thereby treating or reducing severity of the cancer in the subject.


SEQUENCE LISTING (54 sequences)

SEQ ID NO: 1 - DNA, 1197 bp, Mouse
tttacccggagtccgggagaagctcttagtcgtgtggtgattgtgcagaccctcgtggaccactgaacaggagtagctatttctttccacccagttcttcttttccactctcagcttgctgtacatgaagtaagaaccatcagagtccaggactggttcagtgttcttgtagtttagctctgttttcccgttgttggtccactccacgtaaatgtcttcaggcatgaagtctgtgaccatgcaggtcagagtgacctgtttcttagtcatctcttcttctggtggaggcaagacatatacctgtggagctcttactgaccctttgggttttgagatggttctctcgatgggcgctgggaggtctttgttgttgaccttgcatttgaactccttgccactcatccagtcctggtgctggatggggagggcactgaccacccggagagtactgttgtaatcctctctatgggtttgtgtctgagctgtgtgtacttccacgttgttcacaaaccagctgatctggacatctgggtcatcctcgctcacatccaccaccacacatgtgactatggggctcagggagatcatgagtacatccttgatctttggagggaagatgaagacggatggtccacccaagaggttaggtgctgggcatttgcatggaggacagggcttgattgtgggccctctgggttgaaaccggccttctggtttgggcgaggggctgggatatcttgttgaggtctccaggattctctctgttaccacgagctctgctccagggctctcctcgatttttgccttggggtgcagggagatggccccacagaggtagatgccactgtcattgcgccgtgtgtcaaggatgttcatgtggaagtcatgcctgttgggcagctgtatgatctggaagcgggcatcctggacgggttggctcaaaccattacagaaggcggcctgtttttcagtctggttgctgggactcaggcggttccagttcagcataagatcctccgaccagttggacaagctgcaggtgaaggtggcatttgctccctctgacactgtgagccaggctgggtagaaggtgagggacctccagggcccattggggacctctagaagccaccctgattgccagctcaactgcagcacagcccaagtgaatgaccagggtacctgccggacccacat

SEQ ID NO: 2 - protein, 399 aa, Mouse
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 9 aa, Artificial Sequence
  Synthetic construct
AALLNSAGL

SEQ ID NO: 4 - protein, 9 aa, Artificial Sequence
  Synthetic construct
AALLNSAVL

SEQ ID NO: 5 - protein, 9 aa, Artificial Sequence
  Synthetic construct
AQLPNDVVL

SEQ ID NO: 6 - protein, 9 aa, Artificial Sequence
  Synthetic construct
AQLANDVVL

SEQ ID NO: 7 - protein, 9 aa, Artificial Sequence
  Synthetic construct
MAPIDHTAM

SEQ ID NO: 8 - protein, 9 aa, Artificial Sequence
  Synthetic construct
MAPIDHTTM

SEQ ID NO: 9 - protein, 9 aa, Artificial Sequence
  Synthetic construct
ASMTNRELM

SEQ ID NO: 10 - protein, 9 aa, Artificial Sequence
  Synthetic construct
ASMTNMELM

SEQ ID NO: 11 - protein, 8 aa, Artificial Sequence
  Synthetic construct
SIIVFNLV

SEQ ID NO: 12 - protein, 8 aa, Artificial Sequence
  Synthetic construct
SIIVFNLL

SEQ ID NO: 13 - protein, 9 aa, Artificial Sequence
  Synthetic construct
SSPDSLHYL

SEQ ID NO: 14 - protein, 9 aa, Artificial Sequence
  Synthetic construct
SSPYSLHYL

SEQ ID NO: 15 - protein, 9 aa, Artificial Sequence
  Synthetic construct
SMTQHLEPI

SEQ ID NO: 16 - protein, 9 aa, Artificial Sequence
  Synthetic construct
IMTQHLEPI

SEQ ID NO: 17 - protein, 9 aa, Artificial Sequence
  Synthetic construct
SAIRSYQDV

SEQ ID NO: 18 - protein, 9 aa, Artificial Sequence
  Synthetic construct
SAIRSYQYV

SEQ ID NO: 19 - protein, 9 aa, Artificial Sequence
  Synthetic construct
VSPVNDVDV

SEQ ID NO: 20 - protein, 9 aa, Artificial Sequence
  Synthetic construct
VSPVNDLDV

SEQ ID NO: 21 - protein, 9 aa, Artificial Sequence
  Synthetic construct
MGGMNRRPI

SEQ ID NO: 22 - protein, 9 aa, Artificial Sequence
  Synthetic construct
MGVMNRRPI

SEQ ID NO: 23 - protein, 9 aa, Artificial Sequence
  Synthetic construct
FMACNLLLV

SEQ ID NO: 24 - protein, 9 aa, Artificial Sequence
  Synthetic construct
FMSCNLLLV

SEQ ID NO: 25 - protein, 10 aa, Artificial Sequence
  Synthetic construct
YMLDLQPETT

SEQ ID NO: 26 - protein, 8 aa, Artificial Sequence
  Synthetic construct
SIINFEKL

SEQ ID NO: 27 - DNA, 20 bp, Artificial Sequence
  Synthetic construct
cggctaccacatccaaggaa

SEQ ID NO: 28 - DNA, 18 bp, Artificial Sequence
  Synthetic construct
gctggaattaccgcggct

SEQ ID NO: 29 - DNA, 18 bp, Artificial Sequence
  Synthetic construct
gctgccgtcattttctgc

SEQ ID NO: 30 - DNA, 18 bp, Artificial Sequence
  Synthetic construct
tctcactggcccgtcatc

SEQ ID NO: 31 - DNA, 22 bp, Artificial Sequence
  Synthetic construct
aaaggaccctgatgctgccaag

SEQ ID NO: 32 - DNA, 22 bp, Artificial Sequence
  Synthetic construct
tgttcggtctcgtgtagggact

SEQ ID NO: 33 - DNA, 22 bp, Artificial Sequence
  Synthetic construct
gctggatgaagcagtggctctt

SEQ ID NO: 34 - DNA, 22 bp, Artificial Sequence
  Synthetic construct
ggtccttcttcagtcggtgtag

SEQ ID NO: 35 - DNA, 22 bp, Artificial Sequence
  Synthetic construct
acaagtcggagaacgtgcagga

SEQ ID NO: 36 - DNA, 22 bp, Artificial Sequence
  Synthetic construct
gaagtggtggatgagcctgttg

SEQ ID NO: 37 - DNA, 23 bp, Artificial Sequence
  Synthetic construct
tgtcttctcagcatcaagcaagg

SEQ ID NO: 38 - DNA, 22 bp, Artificial Sequence
  Synthetic construct
ttcggacaggtccttcacaacc

SEQ ID NO: 39 - DNA, 23 bp, Artificial Sequence
  Synthetic construct
gaccagtttgtgaaggagcactc

SEQ ID NO: 40 - DNA, 22 bp, Artificial Sequence
  Synthetic construct
acttcaggaggtgcctctacga

SEQ ID NO: 41 - DNA, 22 bp, Artificial Sequence
  Synthetic construct
aacctcctggatgacatgcctg

SEQ ID NO: 42 - DNA, 22 bp, Artificial Sequence
  Synthetic construct
tcgtcccagatgccccgttaaa

SEQ ID NO: 43 - DNA, 22 bp, Artificial Sequence
  Synthetic construct
gggaatggtgaagaccgtgtca

SEQ ID NO: 44 - DNA, 22 bp, Artificial Sequence
  Synthetic construct
cccttgccttattctaccgacg

SEQ ID NO: 45 - DNA, 22 bp, Artificial Sequence
  Synthetic construct
tcaggtgtggaagatgcggttc

SEQ ID NO: 46 - DNA, 22 bp, Artificial Sequence
  Synthetic construct
gagttctcggtactgacgggaa

SEQ ID NO: 47 - DNA, 23 bp, Artificial Sequence
  Synthetic construct
cggtttcaaggcatggtcattgg

SEQ ID NO: 48 - DNA, 22 bp, Artificial Sequence
  Synthetic construct
ccttcgttcctgctgtgagact

SEQ ID NO: 49 - DNA, 22 bp, Artificial Sequence
  Synthetic construct
tgcggactacaagcgaatcacg

SEQ ID NO: 50 - DNA, 22 bp, Artificial Sequence
  Synthetic construct
gctcccaataggtcttcgactc

SEQ ID NO: 51 - DNA, 1149 bp, Homo sapiens
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccacgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 52 - protein, 382 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - DNA, 435 bp, Homo sapiens
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 54 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE